SEQUENCE LISTING (320 sequences)

SEQ ID NO: 1 - protein, 123 aa, artificial sequence
  Re5A08
GSQVQLVESGGGLVQAGGSLRLSCTASGHTFTANRMGWFRQAPGKEREFVAAINWGGDSTNYVDSVKGRFTISRDIAKNTVYLQMNSLKPEDTAVYFCAARNHVTGEFDSWGQGTQVTVSSTS

SEQ ID NO: 2 - protein, 9 aa, artificial sequence
  Re5A08 CDR1
HTFTANRMG

SEQ ID NO: 3 - protein, 15 aa, artificial sequence
  Re5A08 CDR2
FVAAINWGGDSTNYV

SEQ ID NO: 4 - protein, 13 aa, artificial sequence
  Re5A08 CDR3
AARNHVTGEFDSW

SEQ ID NO: 5 - protein, 124 aa, artificial sequence
  Re5B06
GSQVQLVESGGGLVQPGGSLRLSCAASGSIRSIYATVWFRQAPGKEHEWVGSITSSNVTTYADSVKGRFTISRDNAKKTVYLQMNSLKPEDTALYYCNVHFASEYSDYAQIQGQGTQVTVSSTS

SEQ ID NO: 6 - protein, 9 aa, artificial sequence
  Re5B06 CDR1
SIRSIYATV

SEQ ID NO: 7 - protein, 14 aa, artificial sequence
  Re5B06 CDR2
WVGSITSSNVTTYA

SEQ ID NO: 8 - protein, 15 aa, artificial sequence
  Re5B06 CDR3
NVHFASEYSDYAQIQ

SEQ ID NO: 9 - protein, 126 aa, artificial sequence
  Re5C01
GSQVQLVESGGGLVQAGGSLRLSCGVSGRTFSSYAMGWFRQAPGKEREFVATISWSGGTTNYAHSVKGRFTISRDNAKNTVYLQMNSLKVEDTAVYYCYAVSSGSDYDGGMDYWGKGTQVTVSSTS

SEQ ID NO: 10 - protein, 9 aa, artificial sequence
  Re5C01 CDR1
RTFSSYAMG

SEQ ID NO: 11 - protein, 15 aa, artificial sequence
  Re5C01 CDR2
FVATISWSGGTTNYA

SEQ ID NO: 12 - protein, 16 aa, artificial sequence
  Re5C01 CDR3
YAVSSGSDYDGGMDYW

SEQ ID NO: 13 - protein, 120 aa, artificial sequence
  Re5C08
GSQVQLVESGGGSVEAGGSLRLSCAASGRTFNDYNMVWFRQAPGKEREFVAAIKWNGGNTSYADSVKGRFAVSRDNAKNTVYLQMNNLKHEDTAEYLCYTVGPEGDYWGQGTQVTVSSTS

SEQ ID NO: 14 - protein, 9 aa, artificial sequence
  Re5C08 CDR1
RTFNDYNMV

SEQ ID NO: 15 - protein, 15 aa, artificial sequence
  Re5C08 CDR2
FVAAIKWNGGNTSYA

SEQ ID NO: 16 - protein, 10 aa, artificial sequence
  Re5C08 CDR3
YTVGPEGDYW

SEQ ID NO: 17 - protein, 131 aa, artificial sequence
  Re5D06
GSQVQLVESGGGLVQPGGSLRLSCAASGITLDYYAIGWFRQAPGKEREGVSRIRSSDGSTNYADSVKGRFTMSRDNAKNTVYLQMNSLKPEDTAVYYCAYGPLTKYGSSWYWPYEYDYWGQGTQVTVSSTS

SEQ ID NO: 18 - protein, 9 aa, artificial sequence
  Re5D06 CDR1
ITLDYYAIG

SEQ ID NO: 19 - protein, 15 aa, artificial sequence
  Re5D06 CDR2
GVSRIRSSDGSTNYA

SEQ ID NO: 20 - protein, 21 aa, artificial sequence
  Re5D06 CDR3
AYGPLTKYGSSWYWPYEYDYW

SEQ ID NO: 21 - protein, 134 aa, artificial sequence
  Re5E03
GSQVQLVESGGGLVQPGGSLRLSCAASGFTLDYYAIGWFRQAPGKEREGVSCISNSDGSTRYADSVKGRFTISRDNAKNTVYLQMNSLKPEDTAVYYCAGGPQTYYSGSYYYTCAEGAMDYWGKGTLVTVSSTS

SEQ ID NO: 22 - protein, 9 aa, artificial sequence
  Re5E03 CDR1
FTLDYYAIG

SEQ ID NO: 23 - protein, 15 aa, artificial sequence
  Re5E03 CDR2
GVSCISNSDGSTRYA

SEQ ID NO: 24 - protein, 24 aa, artificial sequence
  Re5E03 CDR3
AGGPQTYYSGSYYYTCAEGAMDYW

SEQ ID NO: 25 - protein, 134 aa, artificial sequence
  Re5E11
GSQVQLVESGGGLVQPGGSLRLSCAASGFTLDYYAIGWFRQAPGKEREGVSCISSSDGRTYYADSVKGRFTISRDNAKNTVYLQMNSLKPEDTAVYYCATAPLTYYSGSWYLTCNSDAMDYWGKGTLVTVSSTS

SEQ ID NO: 26 - protein, 9 aa, artificial sequence
  Re5E11 CDR1
FTLDYYAIG

SEQ ID NO: 27 - protein, 15 aa, artificial sequence
  Re5E11 CDR2
GVSCISSSDGRTYYA

SEQ ID NO: 28 - protein, 24 aa, artificial sequence
  Re5E11 CDR3
ATAPLTYYSGSWYLTCNSDAMDYW

SEQ ID NO: 29 - protein, 118 aa, artificial sequence
  Re5F10
GSQVQLVESGGGLVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTS

SEQ ID NO: 30 - protein, 9 aa, artificial sequence
  Re5F10 CDR1
FTFSSFAMG

SEQ ID NO: 31 - protein, 15 aa, artificial sequence
  Re5F10 CDR2
WVATITITGGSTNYA

SEQ ID NO: 32 - protein, 8 aa, artificial sequence
  Re5F10 CDR3
NPDPGCRR

SEQ ID NO: 33 - protein, 117 aa, artificial sequence
  Re5F11
GSQVQLVESGGALVQPGGSLRLSCATSGSISSYRMGWYRQGPGKQRELVAFITIGGITDYIDSVKGRFTISRDNAKNTMYLQMNSLKPEDTAVYYCNADPPLFNWGQGTQVTVSSTS

SEQ ID NO: 34 - protein, 8 aa, artificial sequence
  Re5F11 CDR1
SISSYRMG

SEQ ID NO: 35 - protein, 14 aa, artificial sequence
  Re5F11 CDR2
LVAFITIGGITDYI

SEQ ID NO: 36 - protein, 9 aa, artificial sequence
  Re5F11 CDR3
NADPPLFNW

SEQ ID NO: 37 - protein, 118 aa, artificial sequence
  Re5G05
GSQVQLVESGGGLVQAGGSLRLSCAASGFTATSYAMGWYRQAPGKECEWVATITSTGGNTNYADSVKGRFTISRDNAKNTVYLQMNSLKPEDTAVYYCNPDPGCDWGQGTQVTVSSTS

SEQ ID NO: 38 - protein, 9 aa, artificial sequence
  Re5G05 CDR1
FTATSYAMG

SEQ ID NO: 39 - protein, 15 aa, artificial sequence
  Re5G05 CDR2
WVATITSTGGNTNYA

SEQ ID NO: 40 - protein, 8 aa, artificial sequence
  Re5G05 CDR3
NPDPGCDW

SEQ ID NO: 41 - protein, 127 aa, artificial sequence
  Re6A11
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTLVTVSSTS

SEQ ID NO: 42 - protein, 9 aa, artificial sequence
  Re6A11 CDR1
RTFSNDALG

SEQ ID NO: 43 - protein, 14 aa, artificial sequence
  Re6A11 CDR2
FVAAINWNSGTYYA

SEQ ID NO: 44 - protein, 18 aa, artificial sequence
  Re6A11 CDR3
AAASDYGLPREDFLYDYW

SEQ ID NO: 45 - protein, 131 aa, artificial sequence
  Re6B02
GSQVQLVESGGALVQPGGSLRLSCVASGFTLDYYAIGWFRQAPGKEREGVSRIRSSDGSTNYADSVKGRFTISRDNAKNTVYLQMNSLKPEDTAVYYCAYGPLTKYGSSWYWPYEYDYWGQGTQVTVSSTS

SEQ ID NO: 46 - protein, 9 aa, artificial sequence
  Re6B02 CDR1
FTLDYYAIG

SEQ ID NO: 47 - protein, 15 aa, artificial sequence
  Re6B02 CDR2
GVSRIRSSDGSTNYA

SEQ ID NO: 48 - protein, 21 aa, artificial sequence
  Re6B02 CDR3
AYGPLTKYGSSWYWPYEYDYW

SEQ ID NO: 49 - protein, 116 aa, artificial sequence
  Re6B06
GSQVQLVESGGGLVQAGGSLRLSCAASGRAFSSAPMSWFRQAPGKEREFVASVSWSGDSTNYADSVKGRFTISRDNAKNTGYLQMNSLKPEDTAVYYCKRGPYWGQGTQVTVSSTS

SEQ ID NO: 50 - protein, 9 aa, artificial sequence
  Re6B06 CDR1
RAFSSAPMS

SEQ ID NO: 51 - protein, 15 aa, artificial sequence
  Re6B06 CDR2
FVASVSWSGDSTNYA

SEQ ID NO: 52 - protein, 6 aa, artificial sequence
  Re6B06 CDR3
KRGPYW

SEQ ID NO: 53 - protein, 131 aa, artificial sequence
  Re6B07
GSQVQLVESGGGLVQPGGSLRLSCAASGFTLDYYAIGWFRQAPGKEREGVSYIRSSDGTTYYADSVKGRFTISRDNAKNTVYLQMNSLKPEDTAVYYCAADEAYYSELGWESPWGWSYWGQGTRVTVSSTS

SEQ ID NO: 54 - protein, 9 aa, artificial sequence
  Re6B07 CDR1
FTLDYYAIG

SEQ ID NO: 55 - protein, 15 aa, artificial sequence
  Re6B07 CDR2
GVSYIRSSDGTTYYA

SEQ ID NO: 56 - protein, 21 aa, artificial sequence
  Re6B07 CDR3
AADEAYYSELGWESPWGWSYW

SEQ ID NO: 57 - protein, 123 aa, artificial sequence
  Re6D06
GSQVQLVESGGGLVQAGASLRLSCAASGRMFGVYRMGWFRQAPGKEREFVAGISTSVGTTNYADSVKGRFTISRDNAKNTVYLQMNSLKPEDTAVYYCAARDPTTYEYDYWGQGTQVTVSSTS

SEQ ID NO: 58 - protein, 9 aa, artificial sequence
  Re6D06 CDR1
RMFGVYRMG

SEQ ID NO: 59 - protein, 15 aa, artificial sequence
  Re6D06 CDR2
FVAGISTSVGTTNYA

SEQ ID NO: 60 - protein, 13 aa, artificial sequence
  Re6D06 CDR3
AARDPTTYEYDYW

SEQ ID NO: 61 - protein, 133 aa, artificial sequence
  Re6D08
GSQVQLVESGGGLVQAGGSLRLSCAASGRTFSSYAMGWFHQAPGKEREFVATINWSGDSTYYADSVKGRFTISRDNAKNTVYLQMNSLKPEDTAVYYCAAVVDPSPTYYSGKYYPPRVEYWGKGTQVTVSSTS

SEQ ID NO: 62 - protein, 9 aa, artificial sequence
  Re6D08 CDR1
RTFSSYAMG

SEQ ID NO: 63 - protein, 15 aa, artificial sequence
  Re6D08 CDR2
FVATINWSGDSTYYA

SEQ ID NO: 64 - protein, 23 aa, artificial sequence
  Re6D08 CDR3
AAVVDPSPTYYSGKYYPPRVEYW

SEQ ID NO: 65 - protein, 120 aa, artificial sequence
  Re6D09
GSQVQLVESGGGSVEAGGSLRLSCAASGRTFNNYNMVWFRQAPGKEREFVAAINWNGGSTSYAASVKGRFAVSIDNAKNTLYLQMNNLKHEDTAEYLCYTVGPEGDYWGQGTQVTVSSTS

SEQ ID NO: 66 - protein, 9 aa, artificial sequence
  Re6D09 CDR1
RTFNNYNMV

SEQ ID NO: 67 - protein, 15 aa, artificial sequence
  Re6D09 CDR2
FVAAINWNGGSTSYA

SEQ ID NO: 68 - protein, 10 aa, artificial sequence
  Re6D09 CDR3
YTVGPEGDYW

SEQ ID NO: 69 - protein, 134 aa, artificial sequence
  Re6E11
GSQVQLVESGGGLVQPGGSLRLSCAASGLTLDYYAIGWFRQAPGKEREGVSCISSRDGSTMYADSVKGRFTISRDNAKNTVYLQMNSLKPEDTAVYYCAATPTTYYSGSYYYTCSPEGYDYWGQGTQVTVSSTS

SEQ ID NO: 70 - protein, 9 aa, artificial sequence
  Re6E11 CDR1
LTLDYYAIG

SEQ ID NO: 71 - protein, 15 aa, artificial sequence
  Re6E11 CDR2
GVSCISSRDGSTMYA

SEQ ID NO: 72 - protein, 24 aa, artificial sequence
  Re6E11 CDR3
AATPTTYYSGSYYYTCSPEGYDYW

SEQ ID NO: 73 - protein, 119 aa, artificial sequence
  Re6F06
GSQVQLVESGGGLVQAGGSLRLSCAASGFTFSNYAMGWYRQAPGKECEFVAVITITGSNTNYADSVKGRFTISRDNAKNTVYLQMNSLKPEDTAVYYCNPDPGCESQGQGTRVTVSSTS

SEQ ID NO: 74 - protein, 9 aa, artificial sequence
  Re6F06 CDR1
FTFSNYAMG

SEQ ID NO: 75 - protein, 15 aa, artificial sequence
  Re6F06 CDR2
FVAVITITGSNTNYA

SEQ ID NO: 76 - protein, 9 aa, artificial sequence
  Re6F06 CDR3
NPDPGCESQ

SEQ ID NO: 77 - protein, 130 aa, artificial sequence
  Re6G03
GSQVQLVESGGGLVQAGGSLRLSCAASGRTFSTYRMAWFRLAPGKEREFVAGINWSDGTTSYKDSVKGRFTISRDNAKNTVYLQMDSLKPEDTAVYYCNAHLSTGQEGPGEYFGMDYWGKGTQVTVSSTS

SEQ ID NO: 78 - protein, 9 aa, artificial sequence
  Re6G03 CDR1
RTFSTYRMA

SEQ ID NO: 79 - protein, 15 aa, artificial sequence
  Re6G03 CDR2
FVAGINWSDGTTSYK

SEQ ID NO: 80 - protein, 20 aa, artificial sequence
  Re6G03 CDR3
NAHLSTGQEGPGEYFGMDYW

SEQ ID NO: 81 - protein, 134 aa, artificial sequence
  Re6H06
GSQVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTLVTVSSTS

SEQ ID NO: 82 - protein, 9 aa, artificial sequence
  Re6H06 CDR1
VTLDYYAIG

SEQ ID NO: 83 - protein, 15 aa, artificial sequence
  Re6H06 CDR2
GVSCTSSSDGSTYYA

SEQ ID NO: 84 - protein, 24 aa, artificial sequence
  Re6H06 CDR3
AVVPQTYYGGKYYSQCTANGMDYW

SEQ ID NO: 85 - protein, 119 aa, artificial sequence
  Re6H10
GSQVQLVESGGGLVQPGGSLRLSCAASGFTFSSYAMGWYRQAPGKECEFVAVITITGGSTNYADSVKGRFTISRDNAKNTVYLQMNSLKPEDTAVYYCNPDPGCRGGGQGTLVTVSSTS

SEQ ID NO: 86 - protein, 9 aa, artificial sequence
  Re6H10 CDR1
FTFSSYAMG

SEQ ID NO: 87 - protein, 15 aa, artificial sequence
  Re6H10 CDR2
FVAVITITGGSTNYA

SEQ ID NO: 88 - protein, 9 aa, artificial sequence
  Re6H10 CDR3
NPDPGCRGG

SEQ ID NO: 89 - protein, 125 aa, artificial sequence
  Re7A01
GSQVQLVESGGGLVQAGGSLRLSCAASGRTFSSYAMGWFRQAPGKEREFVATISFSGSTSYAGHVKGRFTISRDNAKNTVYLQMNSLKPEDTAVYYCHAVTRASDQDGGMDYWGQGTQVTVSSTS

SEQ ID NO: 90 - protein, 9 aa, artificial sequence
  Re7A01 CDR1
RTFSSYAMG

SEQ ID NO: 91 - protein, 14 aa, artificial seq
  Re7A01 CDR2
FVATISFSGSTSYA

SEQ ID NO: 92 - protein, 16 aa, artificial sequence
  Re7A01 CDR3
HAVTRASDQDGGMDYW

SEQ ID NO: 93 - protein, 131 aa, artificial sequence
  Re7B01
GSQVQLVESGGGLVQPGGSLRLSCGASGFTLDYYAIGWFRQAPGKEREGVSRIRSNDGSTDYADSVKGRFTISRDNAKNTVYLQMNSLKPEDTAVYYCAYGPLTKYGSSWYWPYEYDYWGQGTQVTVSSTS

SEQ ID NO: 94 - protein, 9 aa, artificial sequence
  Re7B01 CDR1
FTLDYYAIG

SEQ ID NO: 95 - protein, 15 aa, artificial sequence
  Re7B01 CDR2
GVSRIRSNDGSTDYA

SEQ ID NO: 96 - protein, 21 aa, artificial sequence
  Re7B01 CDR3
AYGPLTKYGSSWYWPYEYDYW

SEQ ID NO: 97 - protein, 126 aa, artificial sequence
  Re7D05
GSQVQLVESGGGLVQAGGSLRLSCAASGRTFSSYAMGWFRQAPGKEREFVATISWSGGSTSYADSVKGRFTISRDNAKNTVYLQMNSLKPEDTAVYYCNAVTHHSDQDGGMDYWGKGTLVTVSSTS

SEQ ID NO: 98 - protein, 9 aa, artificial sequence
  Re7D05 CDR1
RTFSSYAMG

SEQ ID NO: 99 - protein, 15 aa, artificial sequence
  Re7D05 CDR2
FVATISWSGGSTSYA

SEQ ID NO: 100 - protein, 16 aa, artificial sequence
  Re7D05 CDR3
NAVTHHSDQDGGMDYW

SEQ ID NO: 101 - protein, 131 aa, artificial sequence
  Re7E02
GSQVQLVESGGGLVQAGGSLRLSCAASGFTLDYYAIGWFRQAPGKEREGVSYIRSSDGTTYYADSVKGRFTISRDNAKNTVYLQMNSLKPEDTAVYYCAADEAYYSELGWESPWGWSYWGQGTQVTVSSTS

SEQ ID NO: 102 - protein, 9 aa, artificial sequence
  Re7E02 CDR1
FTLDYYAIG

SEQ ID NO: 103 - protein, 15 aa, artificial sequence
  Re7E02 CDR2
GVSYIRSSDGTTYYA

SEQ ID NO: 104 - protein, 21 aa, artificial sequence
  Re7E02 CDR3
AADEAYYSELGWESPWGWSYW

SEQ ID NO: 105 - protein, 116 aa, artificial sequence
  Re7H02
GSQVQLVESGGGLVQAGGSLRLSCAASGRAFESAPMSWFRQAPGKEREFVASVSWSGDSTNYADSVKGRFTISRDNAENTGYLQMNSLKPEDTAVYYCKRGPYWGQGTQVTVSSTS

SEQ ID NO: 106 - protein, 9 aa, artificial sequence
  Re7H02 CDR1
RAFESAPMS

SEQ ID NO: 107 - protein, 15 aa, artificial sequence
  Re7H02 CDR2
FVASVSWSGDSTNYA

SEQ ID NO: 108 - protein, 6 aa, artificial sequence
  Re7H02 CDR3
KRGPYW

SEQ ID NO: 109 - protein, 121 aa, artificial sequence
  Re8A03
GSQVQLVESGGGLVQPGGSLRLSCAASGRITGFNGMGWYRQTPGKQRELVASITNGGITKYADSVKGRFTISRDNAKNTVYLQMNSLKPEDTAVYLCYFWRPEFPNLYWGQGTQVTVSSTS

SEQ ID NO: 110 - protein, 9 aa, artificial sequence
  Re8A03 CDR1
RITGFNGMG

SEQ ID NO: 111 - protein, 14 aa, artificial sequence
  Re8A03 CDR2
LVASITNGGITKYA

SEQ ID NO: 112 - protein, 12 aa, artificial sequence
  Re8A03 CDR3
YFWRPEFPNLYW

SEQ ID NO: 113 - protein, 123 aa, artificial sequence
  Re8A06
GSQVQLVESGGGLVQAGGSLRLSCAASGSIFSINAMGWYRQAPGKERELVAAMGSSGWINYADSVKGRLTISRDNAKNTLYLQMNSLKPEDTAVYYCRGTGGVGPTSADYWGQGTQVTVSSTS

SEQ ID NO: 114 - protein, 9 aa, artificial sequence
  Re8A06 CDR1
SIFSINAMG

SEQ ID NO: 115 - protein, 14 aa, artificial sequence
  Re8A06 CDR2
LVAAMGSSGWINYA

SEQ ID NO: 116 - protein, 14 aa, artificial sequence
  Re8A06 CDR3
RGTGGVGPTSADYW

SEQ ID NO: 117 - protein, 128 aa, artificial sequence
  Re8C06
GSQVQLVESGGGLVQAGGSLRLSCAASGRTDTIYNMGWFRQAPGKEREFVAAISWSDGKTTFADSVKGRFTISRDNAKNTVYLQMNSLKPEDTANYYCAAKAFLVAGRSLEEYDYSGQGTQVTVSSTS

SEQ ID NO: 118 - protein, 9 aa, artificial sequence
  Re8C06 CDR1
RTDTIYNMG

SEQ ID NO: 119 - protein, 15 aa, artificial sequence
  Re8C06 CDR2
FVAAISWSDGKTTFA

SEQ ID NO: 120 - protein, 18 aa, artificial sequence
  Re8C06 CDR3
AAKAFLVAGRSLEEYDYS

SEQ ID NO: 121 - protein, 123 aa, artificial sequence
  Re8E12
GSQVQLVESGGGSVQPGGSLRLSCKVSGFTSDVDLRNYLVSWNRQAPGKERELVAAITPTVISGGNTNYADSVKGRFTISRDYSKSTVYLQMNSLNPEDTAVYYCKVGVYWGQGTQVTVSSTS

SEQ ID NO: 122 - protein, 13 aa, artificial sequence
  Re8E12 CDR1
FTSDVDLRNYLVS

SEQ ID NO: 123 - protein, 18 aa, artificial sequence
  Re8E12 CDR2
LVAAITPTVISGGNTNYA

SEQ ID NO: 124 - protein, 6 aa, artificial sequence
  Re8E12 CDR3
KVGVYW

SEQ ID NO: 125 - protein, 123 aa, artificial sequence
  Re8F03
GSQVQLVESGGGLVQPGGSLTLSCKVSGLTSYVDLRNYLVSWYRQGPGKERELVAAITPTAITGGSTNYADSVKGRFTISRDYSKSTVYLQMNSLNPEDTAVYSCKVGVYWGQGTQVTVSSTS

SEQ ID NO: 126 - protein, 13 aa, artificial sequence
  Re8F03 CDR1
LTSYVDLRNYLVS

SEQ ID NO: 127 - protein, 18 aa, artificial sequence
  Re8F03 CDR2
LVAAITPTAITGGSTNYA

SEQ ID NO: 128 - protein, 6 aa, artificial sequence
  Re8F03 CDR3
KVGVYW

SEQ ID NO: 129 - protein, 134 aa, artificial sequence
  Re9B09
GSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSSTS

SEQ ID NO: 130 - protein, 9 aa, artificial sequence
  Re9B09 CDR1
FTLDYYAIG

SEQ ID NO: 131 - protein, 15 aa, artificial sequence
  Re9B09 CDR2
GVSRISSSDGSTDYA

SEQ ID NO: 132 - protein, 24 aa, artificial sequence
  Re9B09 CDR3
ATVPGTYYSGNWYYTWHPEAVDYW

SEQ ID NO: 133 - protein, 123 aa, artificial sequence
  Re9B10
GSQVQLVESGGGLVQPGGSLRLSCAASGRMFGVYRMGWFRQAPGKEREFVAGISTSVGTTNYADSVKGRFTISRDNAKNTVYLQMNSLKPEDTAVYYCAARDPTTYEYDYWGQGTQVTVSSTS

SEQ ID NO: 134 - protein, 9 aa, artificial sequence
  Re9B10 CDR1
RMFGVYRMG

SEQ ID NO: 135 - protein, 15 aa, artificial sequence
  Re9B10 CDR2
FVAGISTSVGTTNYA

SEQ ID NO: 136 - protein, 13 aa, artificial sequence
  Re9B10 CDR3
AARDPTTYEYDYW

SEQ ID NO: 137 - protein, 131 aa, artificial sequence
  Re9C07
GSQVQLVESGGGLVQAGGSLRLSCAASGRTFSRYAMGWFRQAPGKEREFVAAITWNADTTYYADSVKGRFTISRDNAKNTVYLQMNSLKPEDTAVYYCAAGGNHYYSRSYYSSLEYDHWGQGTQVTVSSTS

SEQ ID NO: 138 - protein, 9 aa, artificial sequence
  Re9C07 CDR1
RTFSRYAMG

SEQ ID NO: 139 - protein, 15 aa, artificial sequence
  Re9C07 CDR2
FVAAITWNADTTYYA

SEQ ID NO: 140 - protein, 21 aa, artificial sequence
  Re9C07 CDR3
AAGGNHYYSRSYYSSLEYDHW

SEQ ID NO: 141 - protein, 121 aa, artificial sequence
  Re9C08
GSQVQLVESGGGLVQPGGSLRLSCAVSGNIFGITAWDWHRQAPGKQRELVAHITSRGDTYYLDSVKGRFAISRDHAKNTLSLQMNSLKPEDTAVYYCYLRTFGPPNDHWGQGTQVTVSSTS

SEQ ID NO: 142 - protein, 9 aa, artificial sequence
  Re9C08 CDR1
NIFGITAWD

SEQ ID NO: 143 - protein, 14 aa, artificial sequence
  Re9C08 CDR2
LVAHITSRGDTYYL

SEQ ID NO: 144 - protein, 12 aa, artificial sequence
  Re9C08 CDR3
YLRTFGPPNDHW

SEQ ID NO: 145 - protein, 129 aa, artificial sequence
  Re9D02
GSQVQLVESGGGLVQAGGSLRLSCAASGRTFSNYAMGWFRQAPGKEREFVAAISWGGDTTYYADSLKGRFTISRDNAKNTVYLQMNSLKPEDTAVYYCAADRGLSYYYDRVTEYDYWGQGTQVTVSSTS

SEQ ID NO: 146 - protein, 9 aa, artificial sequence
  Re9D02 CDR1
RTFSNYAMG

SEQ ID NO: 147 - protein, 15 aa, artificial sequence
  Re9D02 CDR2
FVAAISWGGDTTYYA

SEQ ID NO: 148 - protein, 19 aa, artificial sequence
  Re9D02 CDR3
AADRGLSYYYDRVTEYDYW

SEQ ID NO: 149 - protein, 121 aa, artificial sequence
  Re9G05
GSQVQLVESGGGLVQPGGSLRLSCAVSGNISSITAWDWHRQAPGKQRELVAHITSRGDTMYLDSVKGRFAISRDHAKNTLSLQMNSLKPEDTAVYYCYLRTFGPPYDYWGQGTQVTVSSTS

SEQ ID NO: 150 - protein, 9 aa, artificial sequence
  Re9G05 CDR1
NISSITAWD

SEQ ID NO: 151 - protein, 14 aa, artificial sequence
  Re9G05 CDR2
LVAHITSRGDTMYL

SEQ ID NO: 152 - protein, 12 aa, artificial sequence
  Re9G05 CDR3
YLRTFGPPYDYW

SEQ ID NO: 153 - protein, 129 aa, artificial sequence
  Re9G12
GSQVQLVESGGGLVQAGGSLRLSCAASGRTFSSYVMGWFRQAPGKEREFVAHISWSGDSTYYADSVKGRFTIFRDNAKNTAYLQMNSLKPEDTAVYYCAADRGASYYYTWASEYNYWGQGTQVTVSSTS

SEQ ID NO: 154 - protein, 9 aa, artificial sequence
  Re9G12 CDR1
RTFSSYVMG

SEQ ID NO: 155 - protein, 15 aa, artificial sequence
  Re9G12 CDR2
FVAHISWSGDSTYYA

SEQ ID NO: 156 - protein, 19 aa, artificial sequence
  Re9G12 CDR3
AADRGASYYYTWASEYNYW

SEQ ID NO: 157 - protein, 123 aa, artificial sequence
  Re9H01
GSQVQLVESGGGLVQAGDSLRLSCAASGNIFSINAMGWYRQAPGKQRELVAFITSRGSTNYTDSVKGRFTISRDTAKDTVYLQMNSLKPEDTAVYFCRGGYSDYDIYFGSWGQGTQVTVSSTS

SEQ ID NO: 158 - protein, 9 aa, artificial sequence
  Re9H01 CDR1
NIFSINAMG

SEQ ID NO: 159 - protein, 14 aa, artificial sequence
  Re9H01 CDR2
LVAFITSRGSTNYT

SEQ ID NO: 160 - protein, 14 aa, artificial sequence
  Re9H01 CDR3
RGGYSDYDIYFGSW

SEQ ID NO: 161 - protein, 117 aa, artificial sequence
  Re10B02
GSQVQLVESGGGLVQPGGSLRLSCATSGSISSYRMGWYRQGPGKQRELVAFITIGGITDYIDSVKGRFTISRDNAKNTMYLQMNSLKPEDTAVYYCNADPPLFNWGQGTQVTVSSTS

SEQ ID NO: 162 - protein, 8 aa, artificial sequence
  Re10B02 CDR1
SISSYRMG

SEQ ID NO: 163 - protein, 14 aa, artificial sequence
  Re10B02 CDR2
LVAFITIGGITDYI

SEQ ID NO: 164 - protein, 9 aa, artificial sequence
  Re10B02 CDR3
NADPPLFNW

SEQ ID NO: 165 - protein, 131 aa, artificial sequence
  Re10B10
GSQVQLVESGGGLVQPGGSLRLSCAASGFTLDYYAIGWFRQAPGKEREGVSRIRSSDGSTTYADSVKGRFTISRDNAKNTVYLQMNSLKPEDTAVYYCAYGPLTKYGSSWYWPYEYDYWGQGTQVTVSSTS

SEQ ID NO: 166 - protein, 9 aa, artificial sequence
  Re10B10 CDR1
FTLDYYAIG

SEQ ID NO: 167 - protein, 15 aa, artificial sequence
  Re10B10 CDR2
GVSRIRSSDGSTTYA

SEQ ID NO: 168 - protein, 21 aa, artificial sequence
  Re10B10 CDR3
AYGPLTKYGSSWYWPYEYDYW

SEQ ID NO: 169 - protein, 131 aa, artificial sequence
  Re10F10
GSQVQLVESGGGLVQPGGSLRLSCAASGFTLDYYAIGWFRQAPGKEREGVSRIRNNDGSTDYADSVKGRFTISRDNAKNTVYLQMNSLKPEDTAVYYCAYGPLTKYGSSWYWPYEYDYWGQGTQVTVSSTS

SEQ ID NO: 170 - protein, 9 aa, artificial sequence
  Re10F10 CDR1
FTLDYYAIG

SEQ ID NO: 171 - protein, 15 aa, artificial sequence
  Re10F10 CDR2
GVSRIRNNDGSTDYA

SEQ ID NO: 172 - protein, 21 aa, artificial sequence
  Re10F10 CDR3
AYGPLTKYGSSWYWPYEYDYW

SEQ ID NO: 173 - protein, 120 aa, artificial sequence
  Re11C10
GSQVQLVESGGGSVEAGGSLRLSCAASGRTLDNYNAVWFRQAPGKEREFVAAINWNGSNTSYGNSVKGRFAVSRDNAKNTVYLQMNNLKHEDTAEYLCYTVGPEGDYWGQGTQVTVSSTS

SEQ ID NO: 174 - protein, 9 aa, artificial sequence
  Re11C10 CDR1
RTLDNYNAV

SEQ ID NO: 175 - protein, 15 aa, artificial sequence
  Re11C10 CDR2
FVAAINWNGSNTSYG

SEQ ID NO: 176 - protein, 10 aa, artificial sequence
  Re11C10 CDR3
YTVGPEGDYW

SEQ ID NO: 177 - protein, 120 aa, artificial sequence
  Re11E11
GSQVQLVESGGGSVEAGGSLRLSCAASGRTFNNYNIVWFRQAPGKEREFVAAINWNGGSTSYANSVKGRFAVSRDNAKNTVYLQMNNLKHEDTAEYLCYTVGPEGDYWGQGTQVTVSSTS

SEQ ID NO: 178 - protein, 9 aa, artificial sequence
  Re11E11 CDR1
RTFNNYNIV

SEQ ID NO: 179 - protein, 15 aa, artificial sequence
  Re11E11 CDR2
FVAAINWNGGSTSYA

SEQ ID NO: 180 - protein, 10 aa, artificial sequence
  Re11E11 CDR3
YTVGPEGDYW

SEQ ID NO: 181 - protein, 126 aa, artificial sequence
  Re11F07
GSQVQLVESGGGLVQAGGSLRLSCAASGRAFSSGTMGWFRQAPGKEREFVATISWSGGSTSYARSVKGRFTISGDNAENTVYLQMNSLKPEDTAVYYCYAVSSGSDYDGGMDYWGKGTLVTVSSTS

SEQ ID NO: 182 - protein, 9 aa, artificial sequence
  Re11F07 CDR1
RAFSSGTMG

SEQ ID NO: 183 - protein, 15 aa, artificial sequence
  Re11F07 CDR2
FVATISWSGGSTSYA

SEQ ID NO: 184 - protein, 16 aa, artificial sequence
  Re11F07 CDR3
YAVSSGSDYDGGMDYW

SEQ ID NO: 185 - protein, 124 aa, artificial sequence
  Re11F11
GSQVQLVESGGGLVQPGGSLRLSCAASGFTFSNYHMSWYRQAPGKGRELVADITSGGDYTHYADSVKGRFTVSRDNPKNTLYLQMNSLKPEDTAVYHCHVRIFGPGFPVDYRGQGTQVTVSSTS

SEQ ID NO: 186 - protein, 9 aa, artificial sequence
  Re11F11 CDR1
FTFSNYHMS

SEQ ID NO: 187 - protein, 15 aa, artificial sequence
  Re11F11 CDR2
LVADITSGGDYTHYA

SEQ ID NO: 188 - protein, 14 aa, artificial sequence
  Re11F11 CDR3
HVRIFGPGFPVDYR

SEQ ID NO: 189 - protein, 121 aa, artificial sequence
  Re11G09
GSQVQLVESGGGLVHTGGSLRLSCAASGSIFNIYRMAWYRQAPGKQREKVAIITTYGLTDYADSVKGRFTISRDNAKNTTYLQMNSLKPDDTAVYYCNTDPPDLGPGYWGQGTQVTVSSTS

SEQ ID NO: 190 - protein, 9 aa, artificial sequence
  Re11G09 CDR1
SIFNIYRMA

SEQ ID NO: 191 - protein, 14 aa, artificial sequence
  Re11G09 CDR2
KVAIITTYGLTDYA

SEQ ID NO: 192 - protein, 12 aa, artificial sequence
  Re11G09 CDR3
NTDPPDLGPGYW

SEQ ID NO: 193 - protein, 129 aa, artificial sequence
  Re11H04
GSQVQLVESGGGLVQPGGSLRLSCAASGFTLDYYTIAWFRQAPGKEREGVSCISGNDGSTYYADSVKGRFTISRDNAKNTVYLQMNSLKPEDTAVYYCAADRGESYYPFRPSEYHYWGQGTQVTVSSTS

SEQ ID NO: 194 - protein, 9 aa, artificial sequence
  Re11H04 CDR1
FTLDYYTIA

SEQ ID NO: 195 - protein, 15 aa, artificial sequence
  Re11H04 CDR2
GVSCISGNDGSTYYA

SEQ ID NO: 196 - protein, 19 aa, artificial sequence
  Re11H04 CDR3
AADRGESYYPFRPSEYHYW

SEQ ID NO: 197 - protein, 116 aa, artificial sequence
  KG4B11
GSQVQLVESGGGLVQAGGSLRLSCAASGRAFESAPMSWFRQAPGKEREFVASVSWSGDSTNYADSVKGRFTISRDNAKNTGYLQMNSLKPEDTAVYYCKRGPYWGQGTQVTVSSTS

SEQ ID NO: 198 - protein, 9 aa, artificial sequence
  KG4B11 CDR1
RAFESAPMS

SEQ ID NO: 199 - protein, 15 aa, artificial sequence
  KG4B11 CDR2
FVASVSWSGDSTNYA

SEQ ID NO: 200 - protein, 6 aa, artificial sequence
  KG4B11 CDR3
KRGPYW

SEQ ID NO: 201 - protein, 131 aa, artificial sequence
  Re5D06R11
GSQVQLVESGGGLVQPGGSLRLSCAASGITLDYYAMGWFREAPGKEREGVARIRSNDGSTNYADSVKGRFTMSRDNAKDTYYLQMNSLEPEDTAVYYCAYGPLTKYGSEWYWPYEYDYWGQGTQVTVSSTS

SEQ ID NO: 202 - protein, 9 aa, artificial sequence
  Re5D06R11 CDR1
ITLDYYAMG

SEQ ID NO: 203 - protein, 15 aa, artificial sequence
  Re5D06R11 CDR2
GVARIRSNDGSTNYA

SEQ ID NO: 204 - protein, 21 aa, artificial sequence
  Re5D06R11 CDR3
AYGPLTKYGSEWYWPYEYDYW

SEQ ID NO: 205 - protein, 131 aa, artificial sequence
  Re5D06R13
GSQVQLVESGGGLVQPGGSLRLSCAASGITLDYYAMGWFREAPGKEREGVARIRSNDGSVNYADSVKGRFTMSRDNAKDTYYLQMNSLEPEDTADYYCAYGPLTKYGSEWYWPYEYDYWGQGTQVTVSSTS

SEQ ID NO: 206 - protein, 9 aa, artificial sequence
  Re5D06R13 CDR1
ITLDYYAMG

SEQ ID NO: 207 - protein, 15 aa, artificial sequence
  Re5D06R13 CDR2
GVARIRSNDGSVNYA

SEQ ID NO: 208 - protein, 21 aa, artificial sequence
  Re5D06R13 CDR3
AYGPLTKYGSEWYWPYEYDYW

SEQ ID NO: 209 - protein, 31 aa, artificial sequence
  Spacer
EGSEGPESSDGSDSTDPGEQGEGADASDGSE

SEQ ID NO: 210 - protein, 57 aa, artificial sequence
  human collagen XVIII NC trimerization domain
GASSGVRLWATRQAMLGQVHEVPEGWLIFVAEQEELYVRVQNGFRKVQLEARTPLPR

SEQ ID NO: 211 - protein, 204 aa, artificial sequence
  Re6B06 ColXVIII trimer
GSQVQLVESGGGLVQAGGSLRLSCAASGRAFSSAPMSWFRQAPGKEREFVASVSWSGDSTNYADSVKGRFTISRDNAKNTGYLQMNSLKPEDTAVYYCKRGPYWGQGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 212 - protein, 204 aa, artificial sequence
  Re7H02 ColXVIII trimer
GSQVQLVESGGGLVQAGGSLRLSCAASGRAFESAPMSWFRQAPGKEREFVASVSWSGDSTNYADSVKGRFTISRDNAENTGYLQMNSLKPEDTAVYYCKRGPYWGQGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 213 - protein, 204 aa, artificial sequence
  KG4B11 ColXVIII trimer
GSQVQLVESGGGLVQAGGSLRLSCAASGRAFESAPMSWFRQAPGKEREFVASVSWSGDSTNYADSVKGRFTISRDNAKNTGYLQMNSLKPEDTAVYYCKRGPYWGQGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 214 - protein, 211 aa, artificial sequence
  Re6D06 ColXVIII trimer
GSQVQLVESGGGLVQAGASLRLSCAASGRMFGVYRMGWFRQAPGKEREFVAGISTSVGTTNYADSVKGRFTISRDNAKNTVYLQMNSLKPEDTAVYYCAARDPTTYEYDYWGQGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 215 - protein, 215 aa, artificial sequence
  Re6A11/Re9F06 ColXVIII trimer
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTLVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 216 - protein, 211 aa, artificial sequence
  Re5A08 ColXVIII trimer
GSQVQLVESGGGLVQAGGSLRLSCTASGHTFTANRMGWFRQAPGKEREFVAAINWGGDSTNYVDSVKGRFTISRDIAKNTVYLQMNSLKPEDTAVYFCAARNHVTGEFDSWGQGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 217 - protein, 130 aa, artificial sequence
  VHH-72 monomer
GSGQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGS

SEQ ID NO: 218 - protein, 217 aa, artificial sequence
  VHH-72 ColXVIII trimer
GSGQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 219 - protein, 23 aa, artificial sequence
  S. cerevisiae Ost1 signal peptide
MRQVWFSWIVGLFLCFFNVSSAA

SEQ ID NO: 220 - protein, 16 aa, artificial sequence
  P. pastoris Ost1 signal peptide
MKFISILFLLIGSVFG

SEQ ID NO: 221 - protein, 19 aa, artificial sequence
  S. pombe Ost1 signal peptide
MLVLKLLLWSIISGLSLAE

SEQ ID NO: 222 - protein, 17 aa, artificial sequence
  C. albicans Ost1 signal peptide
MWKFFITLGVIFSICSA

SEQ ID NO: 223 - protein, 66 aa, artificial sequence
  propeptide of S. cerevisae alpha-factor
APVNTTTEDETAQIPAEAVIGYSDLEGDFDVAVLPFSNSTNNGLLFINTTIASIAAKEEGVSLEKR

SEQ ID NO: 224 - protein, 131 aa, artificial sequence
  Re5D06R15
GSQVQLVESGGGLVQPGGSLRLSCAASGITLDYYAMGWFREAPGKEREGVARIRNSDGSTNYADSVKGRFTMSRDNAKDTYYLQMNSLEPEDTADYYCAYGPLTKYGSSWYWPYEYDYWGQGTQVTVSSTS

SEQ ID NO: 225 - protein, 9 aa, artificial sequence
  Re5D06R15 CDR1
ITLDYYAMG

SEQ ID NO: 226 - protein, 13 aa, artificial sequence
  Re5D06R15 CDR2
ARIRNSDGSTNYA

SEQ ID NO: 227 - protein, 21 aa, artificial sequence
  Re5D06R15 CDR3
AYGPLTKYGSSWYWPYEYDYW

SEQ ID NO: 228 - protein, 131 aa, artificial sequence
  Re5D06R23
GSQVQLVESGGGLVQPGGSLRLSCAISGSTLDYYAMGWFREAPGKEREGVARIRNNDGSTDYADSVKGRFTMSRDNAKDTYYLQMNSLEPEDTADYYCAYGPLTKYGSSWYWPYEYDYWGQGTQVTVSSTS

SEQ ID NO: 229 - protein, 9 aa, artificial sequence
  Re5D06R23 CDR1
STLDYYAMG

SEQ ID NO: 230 - protein, 13 aa, artificial sequence
  Re5D06R23 CDR2
ARIRNNDGSTDYA

SEQ ID NO: 231 - protein, 21 aa, artificial sequence
  Re5D06R23 CDR3
AYGPLTKYGSSWYWPYEYDYW

SEQ ID NO: 232 - protein, 131 aa, artificial sequence
  Re5D06R28
GSQVQLVESGGGLVQPGGSLRLSCAISGSTLDYYAMGWFREAPGKEREGVARWRNNDGSTNYADSVKGRFTMSRDNAKDTYYLQMNSLEPEDTADYYCAYGPLTKYGSSWHWPYEYDYWGQGTQVTVSSTS

SEQ ID NO: 233 - protein, 9 aa, artificial sequence
  Re5D06R28 CDR1
STLDYYAMG

SEQ ID NO: 234 - protein, 13 aa, artificial sequence
  Re5D06R28 CDR2
ARWRNNDGSTNYA

SEQ ID NO: 235 - protein, 21 aa, artificial sequence
  Re5D06R28 CDR3
AYGPLTKYGSSWHWPYEYDYW

SEQ ID NO: 236 - protein, 131 aa, artificial sequence
  Re5D06R28D
GSQVQLVESGGGLVQPGGSLRLSCAISGSTLDYYAMGWFREAPGKEREGVARWRNNDGSTNYADSVKGRFTMSRDNAKDTYYLQMNSLEPEDTADYYCAYGPLTKYGDEWHWPYEYDYWGQGTQVTVSSTS

SEQ ID NO: 237 - protein, 9 aa, artificial sequence
  Re5D06R28D CDR1
STLDYYAMG

SEQ ID NO: 238 - protein, 13 aa, artificial sequence
  Re5D06R28D CDR2
ARWRNNDGSTNYA

SEQ ID NO: 239 - protein, 21 aa, artificial sequence
  Re5D06R28D CDR3
AYGPLTKYGSSWHWPYEYDYW

SEQ ID NO: 240 - protein, 131 aa, artificial sequence
  Re5D06R15_3QE
GSQVQLVESGGGLVEPGGSLRLSCAASGITLDYYAMGWFREAPGKEREGVARIRNSDGSTNYADSVKGRFTMSRDNAKDTYYLQMNSLEPEDTADYYCAYGPLTKYGSSWYWPYEYDYWGEGTEVTVSSTS

SEQ ID NO: 241 - protein, 9 aa, artificial sequence
  Re5D06R15_3QE CDR1
ITLDYYAMG

SEQ ID NO: 242 - protein, 13 aa, artificial sequence
  Re5D06R15_3QE CDR2
ARIRNSDGSTNYA

SEQ ID NO: 243 - protein, 21 aa, artificial sequence
  Re5D06R15_3QE CDR3
AYGPLTKYGSSWYWPYEYDYW

SEQ ID NO: 244 - protein, 131 aa, artificial sequence
  Re5D06R28_3QE
GSQVQLVESGGGLVEPGGSLRLSCAISGSTLDYYAMGWFREAPGKEREGVARWRNNDGSTNYADSVKGRFTMSRDNAKDTYYLQMNSLEPEDTADYYCAYGPLTKYGSSWHWPYEYDYWGEGTEVTVSSTS

SEQ ID NO: 245 - protein, 9 aa, artificial sequence
  Re5D06R28_3QE CDR1
STLDYYAMG

SEQ ID NO: 246 - protein, 13 aa, artificial sequence
  Re5D06R28_3QE CDR2
ARWRNNDGSTNYA

SEQ ID NO: 247 - protein, 21 aa, artificial sequence
  Re5D06R28_3QE CDR3
AYGPLTKYGSSWHWPYEYDYW

SEQ ID NO: 248 - protein, 127 aa, artificial sequence
  Re9F06
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTS

SEQ ID NO: 249 - protein, 9 aa, artificial sequence
  Re9F06 CDR1
RTFSNDALG

SEQ ID NO: 250 - protein, 12 aa, artificial sequence
  Re9F06 CDR2
AAINWNSGTYYA

SEQ ID NO: 251 - protein, 18 aa, artificial sequence
  Re9F06 CDR3
AAASDYGLPREDFLYDYW

SEQ ID NO: 252 - protein, 134 aa, artificial sequence
  Re9H03
GSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPKAVDYWGKGTLVTVSSTS

SEQ ID NO: 253 - protein, 9 aa, artificial sequence
  Re9H03 CDR1
FTLDYYAIG

SEQ ID NO: 254 - protein, 13 aa, artificial sequence
  Re9H03 CDR2
SRISSSDGSTDYA

SEQ ID NO: 255 - protein, 24 aa, artificial sequence
  Re9H03 CDR3
ATVPGTYYSGNWYYTWHPKAVDYW

SEQ ID NO: 256 - protein, 130 aa, artificial sequence
  Re21B09
GSQVQLVESGGGLVQPGGSLRLSCAASGFTLDNYAIGWFRQAPGKEREGVSCIRSSDGSTYYADSVKGRFTISKDNAKNTVYLQMNSLKPEDTAVYYCATDGTFNPPCDDLYSWYFPERQGTQVTVSSTS

SEQ ID NO: 257 - protein, 9 aa, artificial sequence
  Re21B09 CDR1
FTLDNYAIG

SEQ ID NO: 258 - protein, 13 aa, artificial sequence
  Re21B09 CDR2
SCIRSSDGSTYYA

SEQ ID NO: 259 - protein, 20 aa, artificial sequence
  Re21B09 CDR3
ATDGTFNPPCDDLYSWYFPE

SEQ ID NO: 260 - protein, 118 aa, artificial sequence
  Re21D01
GSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTS

SEQ ID NO: 261 - protein, 9 aa, artificial sequence
  Re21D01 CDR1
FTFSSFAMG

SEQ ID NO: 262 - protein, 13 aa, artificial sequence
  Re21D01 CDR2
ATITITGGSTNYA

SEQ ID NO: 263 - protein, 8 aa, artificial sequence
  Re21D01 CDR3
NPDPGCRR

SEQ ID NO: 264 - protein, 119 aa, artificial sequence
  Re21H01
GSQVQLVESGGALVQPGGSLRLSCVASGFTFSSFAMGWYRQAPGKECEWVATITITGGSTNYADSVKGRFTISRDNAKNTLYLQMNSLKPEDTAVYYCNPDPGCRGGGQGTQVTVSSTS

SEQ ID NO: 265 - protein, 9 aa, artificial sequence
  Re21H01 CDR1
FTFSSFAMG

SEQ ID NO: 266 - protein, 13 aa, artificial sequence
  Re21H01 CDR2
ATITITGGSTNYA

SEQ ID NO: 267 - protein, 9 aa, artificial sequence
  Re21H01 CDR3
NPDPGCRGG

SEQ ID NO: 268 - protein, 127 aa, artificial sequence
  Re22D04
GSQVQLVESGGGLVQTGGSLRLSCAASGRTFSDDAMGWFRQAPGKERDVVAALGWAGVSTYYADSVKGRFGISRDNAKNTVYLQMSSLKPEDTAVYYCAAAPSVAHARLGEWAYWGKGTQVTVSSTS

SEQ ID NO: 269 - protein, 9 aa, artificial sequence
  Re22D04 CDR1
RTFSDDAMG

SEQ ID NO: 270 - protein, 13 aa, artificial sequence
  Re22D04 CDR2
AALGWAGVSTYYA

SEQ ID NO: 271 - protein, 17 aa, artificial sequence
  Re22D04 CDR3
AAAPSVAHARLGEWAYW

SEQ ID NO: 272 - protein, 134 aa, artificial sequence
  Re22E05
GSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPKAVDYWGKGTQVTVSSTS

SEQ ID NO: 273 - protein, 9 aa, artificial sequence
  Re22E05 CDR1
FTLDYYAIG

SEQ ID NO: 274 - protein, 13 aa, artificial sequence
  Re22E05 CDR2
SRISSSDGSTDYA

SEQ ID NO: 275 - protein, 24 aa, artificial sequence
  Re22E05 CDR3
ATVPGTYYSGNWYYTWHPKAVDYW

SEQ ID NO: 276 - protein, 126 aa, artificial sequence
  Re25H10
GSQVQLVESGGGLVQPGGSLRLSCAASGFTFSSSAMSWARQAPGKGLEWVSTISEDGSTYYADSMKGRFTISRDNAKNTVYLQMNSLKPEDTAAYYCATSTEPRTVVAGWGDYLGQGTQVTVSSTS

SEQ ID NO: 277 - protein, 9 aa, artificial sequence
  Re25H10 CDR1
FTFSSSAMS

SEQ ID NO: 278 - protein, 12 aa, artificial sequence
  Re25H10 CDR2
STISEDGSTYYA

SEQ ID NO: 279 - protein, 17 aa, artificial sequence
  Re25H10 CDR3
ATSTEPRTVVAGWGDYL

SEQ ID NO: 280 - protein, 134 aa, artificial sequence
  Re26D07
GSQVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTQVTVSSTS

SEQ ID NO: 281 - protein, 9 aa, artificial sequence
  Re26D07 CDR1
VTLDYYAIG

SEQ ID NO: 282 - protein, 13 aa, artificial sequence
  Re26D07 CDR2
SCTSSSDGSTYYA

SEQ ID NO: 283 - protein, 24 aa, artificial sequence
  Re26D07 CDR3
AVVPQTYYGGKYYSQCTANGMDYW

SEQ ID NO: 284 - protein, 118 aa, artificial sequence
  Re26E09
GSQVQLVESGGALVQPGGSLRLSCVASGFTFSSFAMGWYRQAPGKECEWVATITITGGNTNYADSVKGRFTISRDNAKNTVYLQMNSLKPEDTAVYYCNPDPGCRRGQGTRVTVSSTS

SEQ ID NO: 285 - protein, 9 aa, artificial sequence
  Re26E09 CDR1
FTFSSFAMG

SEQ ID NO: 286 - protein, 13 aa, artificial sequence
  Re26E09 CDR2
ATITITGGNTNYA

SEQ ID NO: 287 - protein, 8 aa, artificial sequence
  Re26E09 CDR3
NPDPGCRR

SEQ ID NO: 288 - protein, 118 aa, artificial sequence
  Re26E11
GSQVQLVESGGALVQPGGSLRLSCVASGFTFSSFAMGWYRQAPGKECEWVATITITGGNTNYADSVKGRFTISRDNAKNTVYLQMNSLKPEDTAVYYCNPDPGCRRGQGTQVTVSSTS

SEQ ID NO: 289 - protein, 9 aa, artificial sequence
  Re26E11 CDR1
FTFSSFAMG

SEQ ID NO: 290 - protein, 13 aa, artificial sequence
  Re26E11 CDR2
ATITITGGNTNYA

SEQ ID NO: 291 - protein, 8 aa, artificial sequence
  Re26E11 CDR3
NPDPGCRR

SEQ ID NO: 292 - protein, 271 aa, artificial sequence
  Re9F06-SpacerA-Re9B09|pDG03599
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGEGGEGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSSTS

SEQ ID NO: 293 - protein, 268 aa, artificial sequence
  Re9F06-SpacerA-Re5D06R28D|pDG03560
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGEGGEGSQVQLVESGGGLVQPGGSLRLSCAISGSTLDYYAMGWFREAPGKEREGVARWRNNDGSTNYADSVKGRFTMSRDNAKDTYYLQMNSLEPEDTADYYCAYGPLTKYGDEWHWPYEYDYWGQGTQVTVSSTS

SEQ ID NO: 294 - protein, 268 aa, artificial sequence
  Re22D04-SpacerA-Re5D06R28D|pD0G3661
GSQVQLVESGGGLVQTGGSLRLSCAASGRTFSDDAMGWFRQAPGKERDVVAALGWAGVSTYYADSVKGRFGISRDNAKNTVYLQMSSLKPEDTAVYYCAAAPSVAHARLGEWAYWGKGTQVTVSSTSGEGEGGEGGEGSQVQLVESGGGLVQPGGSLRLSCAISGSTLDYYAMGWFREAPGKEREGVARWRNNDGSTNYADSVKGRFTMSRDNAKDTYYLQMNSLEPEDTADYYCAYGPLTKYGDEWHWPYEYDYWGQGTQVTVSSTS

SEQ ID NO: 295 - protein, 270 aa, artificial sequence
  Re25H10-SpacerA-Re9B09|pDG03697
GSQVQLVESGGGLVQPGGSLRLSCAASGFTFSSSAMSWARQAPGKGLEWVSTISEDGSTYYADSMKGRFTISRDNAKNTVYLQMNSLKPEDTAAYYCATSTEPRTVVAGWGDYLGQGTQVTVSSTSGEGEGGEGGEGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSSTS

SEQ ID NO: 296 - protein, 267 aa, artificial sequence
  Re25H10-SpacerA-Re5D06R28D|pDG03798
GSQVQLVESGGGLVQPGGSLRLSCAASGFTFSSSAMSWARQAPGKGLEWVSTISEDGSTYYADSMKGRFTISRDNAKNTVYLQMNSLKPEDTAAYYCATSTEPRTVVAGWGDYLGQGTQVTVSSTSGEGEGGEGGEGSQVQLVESGGGLVQPGGSLRLSCAISGSTLDYYAMGWFREAPGKEREGVARWRNNDGSTNYADSVKGRFTMSRDNAKDTYYLQMNSLEPEDTADYYCAYGPLTKYGDEWHWPYEYDYWGQGTQVTVSSTS

SEQ ID NO: 297 - protein, 268 aa, artificial sequence
  Pp086|Re9F06-SpacerB-Re5D06R28D|pDG03637
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGEGSQVQLVESGGGLVQPGGSLRLSCAISGSTLDYYAMGWFREAPGKEREGVARWRNNDGSTNYADSVKGRFTMSRDNAKDTYYLQMNSLEPEDTADYYCAYGPLTKYGDEWHWPYEYDYWGQGTQVTVSS

SEQ ID NO: 298 - protein, 285 aa, artificial sequence
  Pp087|Re9F06-SpacerC-Re6H06|pDG03625
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGSGEGGSEGGEGGSGEGSQVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTLVTVSS

SEQ ID NO: 299 - protein, 285 aa, artificial sequence
  Pp088|Re9F06-SpacerC-Re9B09|pDG03626
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGSGEGGSEGGEGGSGEGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSS

SEQ ID NO: 300 - protein, 271 aa, artificial sequence
  Pp089|Re9F06-SpacerB-Re6H06|pDG03627
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGEGSQVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTLVTVSS

SEQ ID NO: 301 - protein, 271 aa, artificial sequence
  Pp090|Re9F06-SpacerB-Re9B09|pDG03628
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGEGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSS

SEQ ID NO: 302 - protein, 268 aa, artificial sequence
  Pp091|Re9F06-SpacerB-Re5D06R15_3QE|pDG03629
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGEGSQVQLVESGGGLVEPGGSLRLSCAASGITLDYYAMGWFREAPGKEREGVARIRNSDGSTNYADSVKGRFTMSRDNAKDTYYLQMNSLEPEDTADYYCAYGPLTKYGSSWYWPYEYDYWGEGTEVTVSS

SEQ ID NO: 303 - protein, 268 aa, artificial sequence
  Pp092|Re9F06-SpacerB-Re5D06R28D_3QE|pDG03630
EGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTSGEGEGGGEGGEGSQVQLVESGGGLVEPGGSLRLSCAISGSTLDYYAMGWFREAPGKEREGVARWRNNDGSTNYADSVKGRFTMSRDNAKDTYYLQMNSLEPEDTADYYCAYGPLTKYGSSWHWPYEYDYWGEGTEVTVSS

SEQ ID NO: 304 - protein, 259 aa, artificial sequence
  Pp093|Re21D01-SpacerB-Re5D06R15_3QE|pDG03663
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGEGSQVQLVESGGGLVEPGGSLRLSCAASGITLDYYAMGWFREAPGKEREGVARIRNSDGSTNYADSVKGRFTMSRDNAKDTYYLQMNSLEPEDTADYYCAYGPLTKYGSSWYWPYEYDYWGEGTEVTVSS

SEQ ID NO: 305 - protein, 259 aa, artificial sequence
  Pp094|Re21D01-SpacerB-Re5D06R28_3QE|pDG03664
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGEGSQVQLVESGGGLVEPGGSLRLSCAISGSTLDYYAMGWFREAPGKEREGVARWRNNDGSTNYADSVKGRFTMSRDNAKDTYYLQMNSLEPEDTADYYCAYGPLTKYGSSWHWPYEYDYWGEGTEVTVSS

SEQ ID NO: 306 - protein, 273 aa, artificial sequence
  Pp095|Re21D01-SpacerC-Re5D06R15_3QE|pDG03665
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGSGEGGSEGGEGGSGEGSQVQLVESGGGLVEPGGSLRLSCAASGITLDYYAMGWFREAPGKEREGVARIRNSDGSTNYADSVKGRFTMSRDNAKDTYYLQMNSLEPEDTADYYCAYGPLTKYGSSWYWPYEYDYWGEGTEVTVSS

SEQ ID NO: 307 - protein, 273 aa, artificial sequence
  Pp096|Re21D01-SpacerC-Re5D06R28_3QE|pDG03666
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGSGEGGSEGGEGGSGEGSQVQLVESGGGLVEPGGSLRLSCAISGSTLDYYAMGWFREAPGKEREGVARWRNNDGSTNYADSVKGRFTMSRDNAKDTYYLQMNSLEPEDTADYYCAYGPLTKYGSSWHWPYEYDYWGEGTEVTVSS

SEQ ID NO: 308 - protein, 262 aa, artificial sequence
  Pp097|Re21D01-SpacerB-Re6H06|pDG03667
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGEGSQVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTLVTVSS

SEQ ID NO: 309 - protein, 262 aa, artificial sequence
  Pp098|Re21D01-SpacerB-Re9B09|pDG03668
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGEGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSS

SEQ ID NO: 310 - protein, 276 aa, artificial sequence
  Pp099|Re21D01-SpacerC-Re6H06|pDG03669
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGSGEGGSEGGEGGSGEGSQVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTLVTVSS

SEQ ID NO: 311 - protein, 276 aa, artificial sequence
  Pp100|Re21D01-SpacerC-Re9B09|pDG03670
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSSTSGEGEGGGEGGSGEGGSEGGEGGSGEGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSS

SEQ ID NO: 312 - protein, 14 aa, artificial sequence
  Spacer A
TSGEGEGGEGGEGS

SEQ ID NO: 313 - protein, 15 aa, artificial sequence
  Spacer B
TSGEGEGGGEGGEGS

SEQ ID NO: 314 - protein, 29 aa, artificial sequence
  Spacer C
TSGEGEGGGEGGSGEGGSEGGEGGSGEGS

SEQ ID NO: 315 - protein, 223 aa, artificial sequence
  ColXV-Re9B09 ColXV trimer
GSEGNLVTAFSNMDDMLQKAHLVIEGTFIYLRDSTEFFIRVRDGWKKLQLGELIPIPAGSEGPESSDGSDSTDPGEQGEGADASDGSEGGSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSSTS

SEQ ID NO: 316 - protein, 222 aa, artificial sequence
  Re9B09-ColXVIII trimer
GSQVQLVESGGGLVQPGGSLRLSCAASGFTLDYYAIGWFRQAPGKEREGVSRISSSDGSTDYADSVKGRFTISRDNAKNTVYLQMNSLKPEDTAVYYCATVPGTYYSGNWYYTWHPEAVDYWGKGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 317 - protein, 211 aa, artificial sequence
  ColXV-Re9H01 trimer
GSEGNLVTAFSNMDDMLQKAHLVIEGTFIYLRDSTEFFIRVRDGWKKLQLGELIPIPAGSEGPESSDGSDSTDPGEQGEGADASDGSEGGSQVQLVESGGGLVQAGDSLRLSCAASGNIFSINAMGWYRQAPGKQRELVAFITSRGSTNYTDSVKGRFTISRDTAKDTVYLQMNSLKPEDTAVYFCRGGYSDYDIYFGSWGQGTQVTVSST

SEQ ID NO: 318 - protein, 211 aa, artificial sequence
  Re9H01-ColXVIII trimer
GSQVQLVESGGGLVQAGDSLRLSCAASGNIFSINAMGWYRQAPGKQRELVAFITSRGSTNYTDSVKGRFTISRDTAKDTVYLQMNSLKPEDTAVYFCRGGYSDYDIYFGSWGQGTQVTVSSTSEGSEGPESSDGSDSTDPGEQGEGADASDGSEGASSGVRLWATRQAMLGQVHEVPEGWLIFVAEQEELYVRVQNGFRKVQLEARTPLPR

SEQ ID NO: 319 - protein, 205 aa, artificial sequence
  ColXV-Re7H02 trimer
GSEGNLVTAFSNMDDMLQKAHLVIEGTFIYLRDSTEFFIRVRDGWKKLQLGELIPIPAGSEGPESSDGSDSTDPGEQGEGADASDGSEGGSQVQLVESGGGLVQAGGSLRLSCAASGRAFESAPMSWFRQAPGKEREFVASVSWSGDSTNYADSVKGRFTISRDNAENTGYLQMNSLKPEDTAVYYCKRGPYWGQGTQVTVSSTS

SEQ ID NO: 320 - protein, 203 aa, artificial sequence
  KG4B11-ColXV trimer
GSQVQLVESGGGLVQAGGSLRLSCAASGRAFESAPMSWFRQAPGKEREFVASVSWSGDSTNYADSVKGRFTISRDNAKNTGYLQMNSLKPEDTAVYYCKRGPYWGQGTQVTVSSTSEGSEGPESSDGSDSTDPGEQGEGADASDGSEGNLVTAFSNMDDMLQKAHLVIEGTFIYLRDSTEFFIRVRDGWKKLQLGELIPIPAD

(19) United States
(12) Patent Application Publication
(10) Pub. No.: US 2023/0287087 A1
(43) Pub. Date: Sep. 14, 2023

(54) THERAPEUTIC AND DIAGNOSTIC VHH ANTIBODIES AGAINST SARS-COV-2 AND METHODS FOR THEIR ENHANCEMENT

(71) Applicant: Max-Planck-Gesellschaft zur Förderung der Wissenschaften e.V., München (DE)

(72) Inventors: Dirk GÖRLICH, Göttingen (DE); Volker CORDES, Göttingen (DE); Thomas GÜTTLER, Göttingen (DE); Philip GUNKEL, Göttingen (DE); Renate REES, Gleichen (DE); Jens KRULL, Friedland/Deiderode (DE); Kathrin GREGOR, Dransfeld (DE); Waltraud TAXER, Northeim (DE); Leonie NEUMANN, Göttingen (DE); Tino PLEINER, Pasedena, CA (US); Bianka MUSSIL, Göttingen (DE); Ulrike TEICHMANN, Hann-Münden (DE); Aksu METIN, Göttingen (DE); Oleh RYMARENKO, Göttingen (DE); Jürgen SCHÜNEMANN, Gelliehausen (DE); Matthias DOBBELSTEIN, Göttingen (DE); Kim Maren STEGMANN, Göttingen (DE); Antje DICKMANNS, Adelebsen-Güntersen (DE)

(73) Assignee: Max-Planck-Gesellschaft zur Förderung der Wissenschaften e.V., München (DE)

(21) Appl. No.: 18/007,236

(22) PCT Filed: Jul. 29, 2021
(86) PCT No.: PCT/EP2021/071310

§ 371 Date: Jan. 27, 2023

(30) Foreign Application Priority Data
Jul. 29, 2020 (EP) 20188451.7
Jan. 12, 2021 (EP) 21151145.6
Jan. 12, 2021 (EP) 21151159.7
Jul. 2, 2021 (EP) 21183420.5

(51) Int. Cl. C07K 16/10; A61P 31/14

(52) U.S. Cl. CPC C07K 16/10; A61P 31/14; C07K 2317/569; C07K 2317/565; C07K 2317/14; C07K 2317/94

(57) ABSTRACT

The present invention pertains in the fields of antibody technology, protein engineering, medicine, pharmacology, infection biology, virology, and medical diagnostics. More specifically, the present disclosure provides VHH antibodies that prevent cell entry of and infection by SARS-CoV-2, a strategy for an enhanced block of the homotrimeric viral spike proteins by symmetry-matching VHH-fusions, implementations of this strategy, as well as VHH antibodies for sensitive detection of SARS-CoV2-infections.

### FIELD OF THE INVENTION

The present invention pertains in the fields of antibody technology, protein engineering, medicine, pharmacology, infection biology, virology, and medical diagnostics. More specifically, the present disclosure provides VHH antibodies that prevent cell entry of and infection by SARS-CoV-2, a strategy for an enhanced block of the homotrimeric viral spike proteins by symmetry-matching VHH-fusions, implementations of this strategy, methods for producing such symmetry-matching fusions, as well as VHH antibodies for sensitive detection of SARS-CoV2-infections. Further, the present disclosure provides homotrimeric VHH antibodies directed against symmetry-matching target structures different from SARS-CoV-2.

### BACKGROUND OF THE INVENTION

The COVID-19 pandemics has plunged the world into a nearly unprecedented medical and economic crisis—mainly because only insufficient means for treatment are available. COVID-19 is caused by SARS-CoV-2—a recently evolved virus belonging to the Coronaviridae. The virus infects the upper airways initially but can also cause pneumonia and more systemic infections with damages to lungs, kidneys and/or the circulatory system. Approximately 2.2% of the WHO-documented infections had a lethal outcome so far, whereby the probability of a severe course increases with the age of the individuals and with risk factors such as obesity, diabetes, and chronic lung or kidney diseases.

SARS-CoV-2 enters cells with the help of its homotrimeric spike protein, which docks to the ACE2 receptor of the host and subsequently mediates fusion between the viral and host cell membranes to allow entry of the viral genomic RNA into the now infected host cell. The host cell is then re-programmed to produce viral proteins, to replicate the viral RNA, and package replicated RNA into the next generation of viral particles, which are then shed in large numbers off the host cell to infect further cells.

The adaptive immune system can control viral infections by a T-cell mediated cytotoxic response that eliminates infected cells and/or by B-cells producing antibodies that bind to the virus's surface and block infection (for a textbook view see: Murphy and Weaver, 2016). Such infection-blocking antibodies are referred to as neutralizing ones. Typically, however, only a small fraction of produced anti-viral antibodies also has neutralizing properties.

Immunoglobulin G (IgG) is not only the most abundant class of antibodies but also the most important one in terms of long-term immunity. IgGs are built from four polypeptide chains, two light and two heavy ones. They contain two antigen-binding sites formed each by one heavy and one light chain. Such a composite binding site can confer a virus-neutralizing activity. In addition, IgGs comprise an Fc fragment, formed only by the two heavy chains. The Fc fragment is a prototypic immune effector domain that can interact with cell surface receptors (called Fc receptors) as well as with components of the complement system.

In some cases, however, antibodies might even help viral entry by an effect called ADE (for antibody-dependent enhancement) that is mediated by Fc-receptor interactions (Kliks et al., 1989; Littaua et al., 1990). Likewise, the immune system might aggravate the condition of patients with COVID-19 by a systemic over-reaction that manifests itself as a cytokine storm (Chen et al., 2020b; Mehta et al., 2020) or an inappropriate complement activation (Magro et al., 2020).

An effective immune response will either prevent a virus from establishing an infection in the first place or reduce, after an initial infection, the virus-load to non-pathogenic levels. An insufficiently controlled SARS-CoV-2 infection, however, might have a lethal outcome. In survivors, it often causes permanent damage (e.g., lung dysfunction) and impairs quality of life. An efficient anti-viral therapy would be highly desirable, ideally with orthogonal lines of treatment that target distinct steps of the viral infection cycle independently.

Remdesivir represents so far one, still experimental line of treatment. This nucleoside analogue inhibits the SARS-CoV-2 RNA polymerase. As of May 2020, clinical studies gave mixed outcomes: from no decrease in mortality to a modest reduction in hospitalization time (Chen et al., 2020a).

Another treatment option for severely infected patients is passive immunization with polyclonal antibodies/blood plasma from individuals who have already recovered from COVID-19 (Duan et al., 2020). This approach is considered as a last resort and generally has several drawbacks, such as the risk of ADE in case of an insufficient neutralization, limited availability of sera from convalescent individuals, inevitable batch-to-batch variability of plasma samples, the risk of transmitting other infectious diseases, and all other potential adverse effects when large amounts of antibodies of mixed specificity are transferred from one person to another.

A more advanced approach is the isolation of monoclonal antibodies from SARS-CoV-2 infected or convalescent patients (Wang et al., 2020). These can be characterized in-depth and produced reproducibly in a recombinant form. This solves some of the issues raised for the plasma-therapy mentioned above. However, monoclonal antibodies are very costly to produce, making it hard to imagine that it will be feasible to scale up production to levels needed for the treatment of large numbers of patients. In addition, immunological side effects, such as aggravating cytokine storms, inappropriate complement activation, or ADE, cannot be ruled out either, mainly due to the Fc region present in human/humanized antibodies.

Virus diagnostics is another important line for dealing with local, epidemic or even pandemic virus infections. Once infected individuals have been identified, they can be isolated from the still healthy part of the population and thereby a spread of the disease be contained. Also, a proper diagnosis is essential for a targeted treatment, for monitoring the course of an infection as well as for monitoring the success of a treatment. A SARS-CoV-2 infection can be diagnosed by three principles: (1) by a detection of viral RNA, e.g., through RT PCR. This is potentially very sensitive and reports acute infections; (2) by a detection of anti-viral antibodies that patients have produced in response to an infection. This allows potentially higher throughputs but yields false negative results at an early stage of infection, allows no conclusions as to the current viral load and cannot distinguish between past and ongoing infections. (3) The third possibility is to detect viral proteins by means of anti-viral antibodies. This way of detection reports on acute infections but is likely of limited sensitivity, at least when bulk detection in ELISA or lateral flow setups is being used, and when patient's antibodies mask the respective epitopes.

Camelids are unusual in that they produce heavy chain-only antibodies, whose simpler antigen-binding domain can easily be cloned and recombinantly expressed in bacteria or yeast (Arbabi Ghahroudi et al., 1997). Such isolated antigen-binding domains are then referred to as nanobodies or VHH antibodies (variable domain of heavy homodimer antibodies). They are just ˜14 kDa in size—about one-tenth of a traditional IgG molecule. They have found numerous useful applications, for example as crystallization chaperones (Rasmussen et al., 2011; Chug et al., 2015), as tools in affinity chromatography, for localizing proteins in cells or as an animal-friendly substitute for secondary antibodies (Pleiner et al., 2015; Pleiner et al., 2018).

### SUMMARY OF THE INVENTION

A first aspect of the invention relates to an VHH antibody recognizing the SARS-CoV-2 spike protein S1 domain, particularly the receptor-binding domain (RBD) of the SARS-CoV-2 S1 domain.

The VHH antibody is selected from the group of VHH antibodies comprising a CDR3 sequence as disclosed herein or a related CDR3 sequence. In certain claims, the VHH antibody is selected from the group of VHH antibodies comprising a combination of CDR1, CDR2 and CDR3 sequences as disclosed herein or a related combination of CDR1, CDR2 and CDR3 sequences. In certain embodiments, the VHH antibody is selected from the group of antibodies comprising a VHH sequence as disclosed herein or a VHH sequence.

In certain embodiments, the SARS-CoV-2-neutralizing VHH antibody is a stable, particularly a thermostable or a hyperthermostable VHH antibody.

In certain embodiments, the VHH antibody as described above is covalently or non-covalently conjugated to a heterologous moiety, which may be selected from a labeling group, a capture group or an effector group.

In certain embodiments, the VHH antibody as described above is fused to a heterologous polypeptide moiety, which may be selected from a multimerization module, e.g. dimerization, trimerization or tetramerization module. In particular embodiments, the multimerization module is trimerization module.

In certain embodiments, the VHH antibody is conjugated to one or several polymer moieties, preferably hydrophilic polymer moieties, such as polyethylene glycol (PEG), to increase the molecular weight of the antibody conjugate and, thus, delay renal clearance. The molecular weight of a polymer moiety may vary over a broad range, for example in the range of about 5 kDa to about 80 kDa. Such coupling may be performed through e.g. amino or carboxyl groups already present in the VHHs and/or through the side chains of engineered lysine, aspartic acid, glutamic acid or cysteine residues and involve known chemistries for forming amide bonds, secondary amine bonds or thioether bonds.

In certain embodiments, the VHH antibody as described above is directed against the SARS-CoV-2 spike protein receptor-binding domain (RBD). In certain embodiments, the VHH antibody is capable of virus neutralization.

In certain embodiments, the SARS-CoV-2-neutralizing VHH antibody is capable of neutralizing a SARS-CoV2 mutant, in particular a SARS-CoV-2 escape mutant. In certain embodiments, the SARS-CoV-2-neutralizing VHH antibody is capable of neutralizing a SARS-CoV2 mutant comprising a spike protein RBD including at least one amino acid substitution in the RBD selected from the group consisting of K417T, K417N, L452R, E484K, and N501Y.

A further aspect of the invention relates to a heterodimeric VHH antibody, particularly a covalently linked VHH heterodimer comprising a first VHH antibody and a second VHH antibody wherein the first VHH antibody and the second VHH antibody bind to different epitopes on the RBD of the SARS-CoV-2 Spike protein. In certain embodiments, the heterodimeric VHH antibody, particularly the covalently linked VHH heterodimer comprises two VHH antibodies that bind to non-overlapping RBD epitopes. In certain embodiments, the first and the second VHH antibody are fused through a linker sequence.

A further aspect of the present invention relates to a set of two or more different VHH antibodies as described above, wherein the different VHH antibodies may recognize different epitopes, particularly different epitopes on the RBD, and more particularly non-overlapping epitopes on the RBD. A VHH antibody including a heterodimeric VHH antibody as described above is suitable for use in medicine, e.g. human medicine, particularly for use in therapy, e.g. in the prevention or treatment of a disorder caused by and/or associated with an infection with SARS-CoV-2, or in diagnostics, e.g. for detecting SARS-CoV-2 virus or viral components in a patient sample, e.g. in a body fluid or tissue sample.

Still a further aspect of the invention relates to a nucleic acid molecule encoding a VHH antibody including a heterodimeric VHH antibody as described above, particularly in operative linkage with a heterologous expression control sequence, a vector comprising said nucleic acid molecule or a recombinant cell or non-human organism transformed or transfected with said nucleic acid molecule or said vector.

Still a further aspect of the invention relates to a method for recombinant production of a VHH antibody including a heterodimeric VHH antibody as described above, comprising cultivating a cell or an organism as described above in a suitable medium and obtaining the VHH antibody from the cell or organism or from the medium.

Still a further aspect of the invention relates to a method for the prevention or treatment of a disorder caused by and/or associated with an infection with SARS-CoV-2, comprising administering an effective dose of the VHH antibody including a heterodimeric VHH antibody as described above or the set of at least two different VHH antibodies as described above to a subject in need thereof, particularly to a human subject infected with SARS-CoV-2 or at risk from being infected with SARS-CoV-2.

A list of VHH antibodies of the present invention and their utility for virus detection and virus neutralization is shown in the following Table 1:

TABLE 1

| | | Spike protein | | |
|---|---|---|---|---|
| | | detection in | | |
| | | transfected | infected | Virus |
| VHH | Anti | cells* | cells** | neutralization |
| Re5A08 | RBD | Yes | Yes | Yes |
| Re5B06 | RBD | Yes | Yes | Yes |
| Re5C01 | RBD | Yes | Yes | Yes |
| Re5C08 | RBD | Yes | weak | Yes |
| Re5D06 | RBD | Yes | Yes | Yes |
| Re5E03 | RBD | ND | ND | Yes |
| Re5E11 | RBD | ND | ND | Yes |
| Re5F10 | RBD | ND | ND | Yes |
| Re5F11 | RBD | ND | ND | Yes |
| Re5G05 | RBD | ND | ND | Yes |
| Re6A11 | RBD | Yes*** | ND | Yes |
| Re6B02 | RBD | Yes | Yes | Yes |
| Re6B06 | RBD | Yes | weak | Yes |
| Re6B07 | RBD | Yes | weak | Yes |
| Re6D06 | RBD | Yes | Yes | Yes |
| Re6D08 | RBD | Yes | ND | Yes |
| Re6D09 | RBD | Yes | Yes | Yes |
| Re6E11 | RBD | ND | ND | Yes |
| Re6F06 | RBD | ND | ND | Yes |
| Re6G03 | RBD | ND | ND | Yes |
| Re6H06 | RBD | ND | ND | Yes |
| Re6H10 | RBD | ND | ND | Yes |
| Re7A01 | RBD | Yes | Yes | Yes |
| Re7B01 | RBD | Yes | Yes | Yes |
| Re7D05 | RBD | Yes | weak | No |
| Re7E02 | RBD | Yes | Yes | Yes |
| Re7H02 | RBD | Yes | Yes | Yes |
| Re8A03 | S1ΔRBD | Yes | weak | ND |
| Re8A06 | S1ΔRBD | Yes | weak | ND |
| Re8C06 | S1ΔRBD | Yes | Yes | ND |
| Re8E12 | S1ΔRBD | Yes | Yes | ND |
| Re8F03 | S1ΔRBD | Yes | Yes | ND |
| Re9B09 | RBD | Yes | Yes | Yes |
| Re9B10 | RBD | Yes | Yes | Yes |
| Re9C07 | RBD | Yes | Yes | Yes |
| Re9C08 | RBD | Yes | Yes | Yes |
| Re9D02 | RBD | weak | weak | Yes |
| Re9G05 | RBD | Yes | Yes | Yes |
| Re9G12 | RBD | Yes | Yes | No ACE2- |
| | | | | block^{#} |
| Re9H01 | RBD | Yes | Yes | Yes |
| Re10B02 | RBD | ND | ND | Yes |
| Re10B10 | RBD | Yes | Yes | Yes |
| Re10F10 | RBD | Yes | Yes | Yes |
| Re11C10 | RBD | Yes | Yes | Yes |
| Re11E11 | RBD | Yes | Yes | Yes |
| Re11F07 | RBD | Yes | Yes | Yes |
| Re11F11 | RBD | Yes | Yes | Yes |
| Re11G09 | RBD | Yes | Yes | Yes |
| Re11H04 | RBD | weak | weak | Yes |
| KG4B11 | RBD | ND | ND | Yes |
| Re5D06R11 | RBD | ND | ND | Yes |
| Re5D06R13 | RBD | ND | ND | Yes |
| Re5D06R15 | RBD | ND | ND | Yes |
| Re5D06R28 | RBD | ND | ND | Yes |
| Re5D06R15_3QE | RBD | ND | ND | Yes |
| Re5D06R28D | RBD | Yes | Yes | Yes |
| Re5D06R28_3QE | RBD | ND | ND | Yes |
| Re9F06 | RBD | Yes | Yes | Yes |
| Re9H03 | RBD | Yes | Yes | Yes |
| Re21B09 | RBD | Yes | Yes | No ACE2- |
| | | | | block^{#} |
| | RBD | ND | ND | Yes |
| Re21H01 | RBD | ND | ND | Yes |
| Re22D04 | RBD | Yes | Yes | No ACE2- |
| | | | | block^{#} |
| Re22E05 | RBD | Yes | Yes | Yes |
| Re25H10 | RBD | Yes | Yes | No ACE2- |
| | | | | block^{#} |
| Re26D07 | RBD | ND | ND | Yes |
| Re26E09 | RBD | ND | ND | Yes |
| Re26E11 | RBD | ND | ND | Yes |

*SARS-COV2 spike protein detection in transiently transfected HeLa cells with 5 minutes fixation with 4% PFA;
**in SARS-COV-2 infected Vero E6 cells with 60 minutes fixation with 4% PFA.
***Sensitive detection only with trimerized VHH.
ND not determined.
^{#}Binding site on the RBD does not overlap with ACE2, i.e. the VHH cannot neutralize by blocking the RBD-ACE2-interaction. However, such VHHs are useful as tandem fusion partners for directly neutralizing VHHs to increase their avidity and mutation-tolerance (see below).

The variable regions (CDRs) of each VHH antibody of Table 1 as well as assignment of SEQ ID NOs. for VHHs and CDRs are listed in the following Table 2:

TABLE 2

| | SEQ | | SEQ | | SEQ | | SEQ |
|---|---|---|---|---|---|---|---|
| | ID | | ID | | ID | | ID |
| VHH | NO. | CDR1 | NO. | CDR2 | NO. | CDR3 | NO. |
| Re5A08 | 1 | HTFTANRMG | 2 | FVAAINWGGDSTNYV | 3 | AARNHVTGEFDSW | 4 |
| Re5B06 | 5 | SIRSIYATV | 6 | WVGSITSSNVTTYA | 7 | NVHFASEYSDYAQIQ | 8 |
| Re5C01 | 9 | RTFSSYAMG | 10 | FVATISWSGGTTNYA | 11 | YAVSSGSDYDGGMDYW | 12 |
| Re5C08 | 13 | RTFNDYNMV | 14 | FVAAIKWNGGNTSYA | 15 | YTVGPEGDYW | 16 |
| Re5D06 | 17 | ITLDYYAIG | 18 | GVSRIRSSDGSTNYA | 19 | AYGPLTKYGSSWYWPYEYDYW | 20 |
| Re5E03 | 21 | FTLDYYAIG | 22 | GVSCISNSDGSTRYA | 23 | AGGPQTYYSGSYYYTCAEGAM | 24 |
| | | | | | | DYW | |
| Re5E11 | 25 | FTLDYYAIG | 26 | GVSCISSSDGRTYYA | 27 | ATAPLTYYSGSWYLTCNSDAM | 28 |
| | | | | | | DYW | |
| Re5F10 | 29 | FTFSSFAMG | 30 | WWVATITITGGSTNY | 31 | NPDPGCRR | 32 |
| | | | | A | | | |
| Re5F11 | 33 | SISSYRMG | 34 | LVAFITIGGITDYI | 35 | NADPPLFNW | 36 |
| Re5G05 | 37 | FTATSYAMG | 38 | WVATITSTGGNTNYA | 39 | NPDPGCDW | 40 |
| Re6A11 | 41 | RTFSNDALG | 42 | FVAAINWNSGTYYA | 43 | AAASDYGLPREDFLYDYW | 44 |
| Re6B02 | 45 | FTLDYYAIG | 46 | GVSRIRSSDGSTNYA | 47 | AYGPLTKYGSSWYWPYEYDYW | 48 |
| Re6B06 | 49 | RAFSSAPMS | 50 | FVASVSWSGDSTNYA | 51 | KRGPYW | 52 |
| Re6B07 | 53 | FTLDYYAIG | 54 | GVSYIRSSDGTTYYA | 55 | AADEAYYSELGWESPWGWSYW | 56 |
| Re6D06 | 57 | RMFGVYRMG | 58 | FVAGISTSVGTTNYA | 59 | AARDPTTYEYDYW | 60 |
| Re6D08 | 61 | RTFSSYAMG | 62 | FVATINWSGDSTYYA | 63 | AAVVDPSPTYYSGKYYPPRVE | 64 |
| | | | | | | YW | |
| Re6D09 | 65 | RTFNNYNMV | 66 | FVAAINWNGGSTSYA | 67 | YTVGPEGDYW | 68 |
| Re6E11 | 69 | LTLDYYAIG | 70 | GVSCISSRDGSTMYA | 71 | AATPTTYYSGSYYYTCSPEGY | 72 |
| | | | | | | DYW | |
| Re6F06 | 73 | FTFSNYAMG | 74 | FVAVITITGSNTNYA | 75 | NPDPGCESQ | 76 |
| Re6G03 | 77 | RTFSTYRMA | 78 | FVAGINWSDGTTSYK | 79 | NAHLSTGQEGPGEYFGMDYW | 80 |
| Re6H06 | 81 | VTLDYYAIG | 82 | GVSCTSSSDGSTYYA | 83 | AVVPQTYYGGKYYSQCTANGM | 84 |
| | | | | | | DYW | |
| Re6H10 | 85 | FTFSSYAMG | 86 | FVAVITITGGSTNYA | 87 | NPDPGCRGG | 88 |
| Re7A01 | 89 | RTFSSYAMG | 90 | FVATISFSGSTSYA | 91 | HAVTRASDQDGGMDYW | 92 |
| Re7B01 | 93 | FTLDYYAIG | 94 | GVSRIRSNDGSTDYA | 95 | AYGPLTKYGSSWYWPYEYDYW | 96 |
| Re7D05 | 97 | RTFSSYAMG | 98 | FVATISWSGGSTSYA | 99 | NAVTHHSDQDGGMDYW | 100 |
| Re7E02 | 101 | FTLDYYAIG | 102 | GVSYIRSSDGTTYYA | 103 | AADEAYYSELGWESPWGWSYW | 104 |
| Re7H02 | 105 | RAFESAPMS | 106 | FVASVSWSGDSTNYA | 107 | KRGPYW | 108 |
| Re8A03 | 109 | RITGFNGMG | 110 | LVASITNGGITKYA | 111 | YFWRPEFPNLYW | 112 |
| Re8A06 | 113 | SIFSINAMG | 114 | LVAAMGSSGWINYA | 115 | RGTGGVGPTSADYW | 116 |
| Re8C06 | 117 | RTDTIYNMG | 118 | FVAAISWSDGKTTFA | 119 | AAKAFLVAGRSLEEYDYS | 120 |
| Re8E12 | 121 | FTSDVDLRN | 122 | LVAAITPTVISGGNT | 123 | KVGVYW | 124 |
| | | YLVS | | NYA | | | |
| Re8F03 | 125 | LTSYVDLRN | 126 | LVAAITPTAITGGST | 127 | KVGVYW | 128 |
| | | YLVS | | NYA | | | |
| Re9B09 | 129 | FTLDYYAIG | 130 | GVSRISSSDGSTDYA | 131 | ATVPGTYYSGNWYYTWHPEAV | 132 |
| | | | | | | DYW | |
| Re9B10 | 133 | RMFGVYRMG | 134 | FVAGISTSVGTTNYA | 135 | AARDPTTYEYDYW | 136 |
| Re9C07 | 137 | RTFSRYAMG | 138 | FVAAITWNADTTYYA | 139 | AAGGNHYYSRSYYSSLEYDHW | 140 |
| Re9C08 | 141 | NIFGITAWD | 142 | LVAHITSRGDTYYL | 143 | YLRTFGPPNDHW | 144 |
| Re9D02 | 145 | RTFSNYAMG | 146 | FVAAISWGGDTTYYA | 147 | AADRGLSYYYDRVTEYDYW | 148 |
| Re9G05 | 149 | NISSITAWD | 150 | LVAHITSRGDTMYL | 151 | YLRTFGPPYDYW | 152 |
| Re9G12 | 153 | RTFSSYVMG | 154 | FVAHISWSGDSTYYA | 155 | AADRGASYYYTWASEYNYW | 156 |
| Re9H01 | 157 | NIFSINAMG | 158 | LVAFITSRGSTNYT | 159 | RGGYSDYDIYFGSW | 160 |
| Re10B02 | 161 | SISSYRMG | 162 | LVAFITIGGITDYI | 163 | NADPPLFNW | 164 |
| Re10B10 | 165 | FTLDYYAIG | 166 | GVSRIRSSDGSTTYA | 167 | AYGPLTKYGSSWYWPYEYDYW | 168 |
| Re10F10 | 169 | FTLDYYAIG | 170 | GVSRIRNNDGSTDYA | 171 | AYGPLTKYGSSWYWPYEYDYW | 172 |
| Re11C10 | 173 | RTLDNYNAV | 174 | FVAAINWNGSNTSYG | 175 | YTVGPEGDYW | 176 |
| Re11E11 | 177 | RTFNNYNIV | 178 | FVAAINWNGGSTSYA | 179 | YTVGPEGDYW | 180 |
| Re11F07 | 181 | RAFSSGTMG | 182 | FVATISWSGGSTSYA | 183 | YAVSSGSDYDGGMDYW | 184 |
| Re11F11 | 185 | FTFSNYHMS | 186 | LVADITSGGDYTHYA | 187 | HVRIFGPGFPVDYR | 188 |
| Re11G09 | 189 | SIFNIYRMA | 190 | KVAIITTYGLTDYA | 191 | NTDPPDLGPGYW | 192 |
| Re11H04 | 193 | FTLDYYTIA | 194 | GVSCISGNDGSTYYA | 195 | AADRGESYYPFRPSEYHYW | 196 |
| KG4B11 | 197 | RAFESAPMS | 198 | FVASVSWSGDSTNYA | 199 | KRGPYW | 200 |
| Re5D06R11 | 201 | ITLDYYAMG | 202 | GVARIRSNDGSTNYA | 203 | AYGPLTKYGSEWYWPYEYDYW | 204 |
| Re5D06R13 | 205 | ITLDYYAMG | 206 | GVARIRSNDGSVNYA | 207 | AYGPLTKYGSEWYWPYEYDYW | 208 |
| Re5D06R15 | 224 | ITLDYYAMG | 225 | ARIRNSDGSTNYA | 226 | AYGPLTKYGSSWYWPYEYDYW | 227 |
| Re5D06R23 | 228 | STLDYYAMG | 229 | ARIRNNDGSTDYA | 230 | AYGPLTKYGSSWYWPYEYDYW | 231 |
| Re5D06R28 | 232 | STLDYYAMG | 233 | ARWRNNDGSTNYA | 234 | AYGPLTKYGSSWHWPYEYDYW | 235 |
| Re5D06R28D | 236 | STLDYYAMG | 237 | ARWRNNDGSTNYA | 238 | AYGPLTKYGSSWHWPYEYDYW | 239 |
| Re5D06R15_3QE | 240 | ITLDYYAMG | 241 | ARIRNSDGSTNYA | 242 | AYGPLTKYGSSWYWPYEYDYW | 243 |
| Re5D06R28_3QE | 244 | STLDYYAMG | 245 | ARWRNNDGSTNYA | 246 | AYGPLTKYGSSWHWPYEYDYW | 247 |
| Re9F06 | 248 | RTFSNDALG | 249 | AAINWNSGTYYA | 250 | AAASDYGLPREDFLYDYW | 251 |
| Re9H03 | 252 | FTLDYYAIG | 253 | SRISSSDGSTDYA | 254 | ATVPGTYYSGNWYYTWHPKAV | 255 |
| | | | | | | DYW | |
| Re21B09 | 256 | FTLDNYAIG | 257 | SCIRSSDGSTYYA | 258 | ATDGTFNPPCDDLYSWYFPE | 259 |
| Re21D01 | 260 | FTFSSFAMG | 261 | ATITITGGSTNYA | 262 | NPDPGCRR | 263 |
| Re21H01 | 264 | FTFSSFAMG | 265 | ATITITGGSTNYA | 266 | NPDPGCRGG | 267 |
| Re22D04 | 268 | RTFSDDAMG | 269 | AALGWAGVSTYYA | 270 | AAAPSVAHARLGEWAYW | 271 |
| Re22E05 | 272 | FTLDYYAIG | 273 | SRISSSDGSTDYA | 274 | ATVPGTYYSGNWYYTWHPKAV | 275 |
| | | | | | | DYW | |
| Re25H10 | 276 | FTFSSSAMS | 277 | STISEDGSTYYA | 278 | ATSTEPRTVVAGWGDYL | 279 |
| Re26D07 | 280 | VTLDYYAIG | 281 | SCTSSSDGSTYYA | 282 | AVVPQTYYGGKYYSQCTANGM | 283 |
| | | | | | | DYW | |
| Re26E09 | 284 | FTFSSFAMG | 285 | ATITITGGNTNYA | 286 | NPDPGCRR | 287 |
| Re26E11 | 288 | FTFSSFAMG | 289 | ATITITGGNTNYA | 290 | NPDPGCRR | 291 |

A list of complete sequences of VHH antibodies is shown at the end of the specification (see also FIG. 1).

A further independent aspect of the present invention relates to a homomultimeric VHH antibody, particularly a homotrimeric VHH antibody. The homomultimeric VHH antibody comprises a plurality of individual subunits, particularly 3 individual subunits. An individual subunit may be a fusion polypeptide comprising a VHH antibody, e.g. a VHH antibody as described above, directly linked or linked via a spacer to a multimerization module, particularly to a trimerization module.

In certain embodiments, an individual subunit may comprise at its N-terminus the VHH antibody and at its C-terminus the multimerization module, optionally linked via a peptide spacer. In other embodiments, an individual subunit may comprise the multimerization module at its N-terminus and the VHH antibody at its C-terminus, optionally linked via spacer.

In certain embodiments, the spacer is a peptide spacer having a length of about 5 to about 100 amino acids, particularly of about 10 to about 50 amino acids.

In certain embodiments, the peptide spacer comprises amino acids selected from glycine, serine, threonine, alanine, proline, glutamate, and aspartate. In certain embodiments, the spacer is composed to at least about 50%, about 60%, about 70%, about 80%, about 90% or completely of these amino acids.

In certain embodiments, the spacer is composed of a succession of tripeptides, whose first two residues are selected from glycine, serine, threonine, alanine and proline, and the third amino acid is selected from glutamate and aspartate. In certain embodiments, each tripeptide differs in at least one amino acid from adjacent tripeptides.

In certain embodiments, the linker is substantially free or free from amino acids selected from basic amino acids lysine and arginine, as well as from one, more or all of amino acids selected from asparagine, tryptophan, tyrosine, phenylalanine, methionine, leucine, isoleucine, valine, histidine and cysteine.

In further embodiments, the linker consists of amino acids selected from glycine, glutamate, and serine.

In certain embodiments, the homomultimeric VHH antibody is a homotrimeric VHH antibody.

In certain embodiments of this aspect, the VHH antibody portion of the homomultimeric VHH antibody is directed to the SARS-CoV-2 spike protein S1 domain, particularly the receptor-binding domain (RBD) of the SARS-CoV-2 S1 domain. In particular embodiments, the VHH antibody portion of the homomultimeric VHH antibody is a VHH antibody as described above, i.e. an antibody comprising a CDR3 sequence as shown in Table 2 or a sequence related thereto, a combination of CDR1, CDR2 and CDR3 sequences, as shown in Table 2 or a related sequence, or a VHH sequence as shown in FIG. 1 or a related sequence.

According to this aspect, the VHH antibody portion may be directed to another target structure, particularly a target structure having a threefold-rotational symmetry, e.g. viral or non-viral target structure comprising a homotrimeric protein or protein assembly.

A further aspect of the invention relates to a VHH antibody, particularly a trimeric VHH antibody, which is produced in a yeast cell, particularly in a Pichia yeast cell, and to a method for producing a VHH antibody, particularly a trimeric VHH antibody in a yeast cell, particularly in a Pichia yeast cell.

A further aspect of the present invention relates to a heterodimeric VHH antibody, particularly a covalently linked VHH heterodimer comprising a first VHH antibody and a second VHH antibody wherein the first VHH antibody and the second VHH antibody bind to different epitopes on the RBD of the SARS-CoV-2 S1 domain.

### EMBODIMENTS OF THE INVENTION

In the following, specific embodiments of the invention are disclosed as follows:
1. A VHH antibody recognizing the SARS-CoV-2 spike protein S1 domain, particularly the receptor-binding domain (RBD) of the SARS-CoV-2 S1 domain comprising
   (a) a CDR3 sequence as shown in SEQ. ID NO: 20, 204, 208, 4, 8, 12, 16, 24, 28, 32, 36, 40, 44, 48, 52, 56, 60, 64, 68, 72, 76, 80, 84, 88, 92, 96, 100, 104,108, 112, 116, 120, 124, 128, 132, 136, 140, 144, 148, 152, 156, 160, 164, 168, 172, 176, 180, 184, 188, 192, 196, 200, 227, 231, 235, 239, 243, 247, 251, 255, 259, 263, 267, 271, 275, 279, 283, 287, or 291,
   (b) a CDR3 sequence, which has an identity of at least 80%, at least 90% or at least 95% to a CDR3 sequence of (a), or
   (c) a VHH antibody, which competes with a VHH antibody of (a) for the binding to the SARS-CoV-2 spike protein S1 domain, particularly the receptor-binding domain (RBD) of the SARS-CoV-2 S1 domain.
2. The VHH antibody according to embodiment 1 comprising
   (a) a combination of CDR1, CDR2 and CDR3 sequences as shown in SEQ. ID NO: 18-20, 202-204, 206-208, 2-4, 6-8, 10-12, 14-16, 22-24, 26-28, 30-32, 34-36, 38-40, 42-44, 46-48, 50-52, 54-56, 58-60, 62-64, 66-68, 70-72, 74-76, 78-80, 82-84, 86-88, 90-92, 94-96, 98-100, 102-104, 106-108, 110-112, 114-116, 118-120, 122-124, 126-128, 130-132, 134-136, 138-140, 142-144, 146-148, 150-152, 154-156, 158-160, 162-164, 166-168, 170-172, 174-176, 178-180, 182-184, 186-188, 190-192, 194-196, 198-200, 225-227, 229-231, 233-235, 237-239, 241-243, 245-247, 249-251, 253-255, 257-259, 261-263, 265-267, 269-271, 273-275, 277-279, 281-283, 285-287, or 289-291,
   (b) a combination of CDR1, CDR2 and CDR3 sequences which has an identity of at least 80%, at least 90% or at least 95% to a combination of CDR1, CDR2 and CDR3 sequences of (a), or
   (c) a VHH antibody, which competes with a VHH antibody of (a) for the binding to the SARS-CoV-2 spike protein S1 domain, particularly the receptor-binding domain (RBD) of the SARS-CoV-2 S1 domain.
3. The VHH antibody according to embodiment 1 or 2 comprising
   (a) a VHH sequence as shown in SEQ. ID NO: 17, 201, 205, 1, 5, 9, 13, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161,165, 169, 173, 177, 181, 185, 189, 193, 197, 224, 228, 232, 236, 240, 244, 248, 252, 256, 260, 264, 268, 272, 276, 280, 284 or 288,
   (b) a sequence which has an identity of at least 70%, at least 80%, at least 90%, at least 95% or at least 99% to a VHH sequence of (a), or
   (c) a VHH antibody, which competes with a VHH antibody of (a) for the binding to the SARS-CoV-2 spike protein S1 domain, particularly the receptor-binding domain (RBD) of the SARS-CoV-2 S1 domain.
4. The VHH antibody of any one of embodiments 1-3, which recognizes the receptor-binding domain (RBD) of the SARS-CoV-2 S1 domain.
5. The VHH antibody of any one of embodiments 1-4, which is capable of virus neutralization.
6. The VHH antibody of any one of embodiments 1-5, which is capable of neutralizing a SARS-CoV2 mutant, in particular a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.
7. The VHH antibody of any one of embodiments 1-6, which is capable of neutralizing a SARS-CoV2 mutant comprising a spike protein RBD including at least one amino acid substitution in the RBD selected from the group consisting of K417T, K417N, L452R, E484K, N501 and T478K.
8. The VHH antibody of any one of embodiments 5-7, which neutralizes SARS-CoV-2 or a SARS-CoV-2 mutant at a concentration of about 500 pM or less, of about 250 pM or less, of about 170 pM or less, of about 100 pM or less, or of about 50 pM or less.
9. The VHH antibody of any one of embodiments 1-8, which is stable, particularly thermostable or hyperthermostable.
10. The VHH antibody of embodiment 9, which has a melting temperature of at least about 65° C., of at least about 80° C., of at least 90° C. or of at least about 95° C. when measured under non-reducing conditions and/or under reducing conditions.
11. The VHH antibody of embodiment 9 or 10, which has an aggregation temperature of at least about 50° C., of at least about 60° C., of at least 70° C. or of at least about 80° C. when measured under non-reducing conditions and/or under reducing conditions.
12. The VHH antibody of any one of embodiments 1-11, which is selected from antibody Re5D06 comprising a VHH sequence as shown in SEQ. ID NO: 17 or a VHH antibody, which is a variant thereof, particularly a variant comprising at least one of the mutations: A261, 129S, 136M, Q41E, S51A, 153W, S55N, S56N, T60V, N61D, N79D, V81Y, K89E, V95D, Y106X1 (with X1 being an amino acid residue selected from D, N, or E), S108X2 (with X2 being any amino acid residue except for C), S109X3 (with X3 being any amino acid residue, in particular E, Q or K, except for C or P), and Y111H, and more particularly a variant as shown in SEQ. ID NO: 201, (Re5D06R11), 205 (Re5D06R13), 224 (Re5D06R15), 228 (Re5D06R23), 232 (Re5D06R28), 236 (Re5D06R28D), 240 (Re5D06R15_3QE), or 244 (Re5D06R28_3QE).
13. The VHH antibody of any one of embodiments 1-11, which is selected from antibody Re9H03 comprising a VHH sequence as shown in SEQ. ID NO: 252 or a VHH antibody, which is a variant thereof, particularly a variant as shown in SEQ. ID NO: 272 (Re22E05).
14. The VHH antibody of any one of embodiments 1-11, which is selected from antibody Re5F10 comprising a VHH sequence as shown in SEQ. ID NO: 29 or a VHH antibody, which is a variant thereof, particularly a variant as shown in SEQ. ID NO: 260 (Re21 D01), 284 (Re26E09), or 288 (Re26E11).
15. The VHH antibody of any one of embodiments 1-11, which is selected from antibody Re21H01 comprising a VHH sequence as shown in SEQ. ID NO: 264 or a VHH antibody, which is a variant thereof.
16. The VHH antibody of any one of embodiments 1-11, which is selected from antibody Re25H10 comprising a VHH sequence as shown in SEQ. ID NO: 276 or a VHH antibody, which is a variant thereof.
17. The VHH antibody of any one of embodiments 1-11, which is selected from antibody Re6H06 comprising a VHH sequence as shown in SEQ. ID NO: 73 or a VHH antibody, which is a variant thereof, particularly a variant as shown in SEQ. ID NO: 280 (Re26D07).
18. The VHH antibody of any one of embodiments 1-17, which is covalently or non-covalently conjugated to a heterologous moiety, e.g. a labeling group, a capture group or an effector group, wherein the heterologous moiety is particularly selected from a fluorescence group, biotin, an enzyme such as a peroxidase, phosphatase or luciferase, a hapten, an affinity tag, or a nucleic acid such as an oligonucleotide.
19. The VHH antibody of any one of embodiments 1-18, which is fused to a heterologous polypeptide moiety.
20. The VHH antibody of embodiment 19, wherein the heterologous polypeptide moiety is a multimerization module, e.g. dimerization, trimerization or tetramerization module.
21. The VHH antibody of embodiment 20, which is a homo-trimerized VHH antibody fused to a trimerization module, e.g. a collagen trimerization moiety, particularly a human collagen moiety, or a lung surfactant protein D moiety.
22. The VHH antibody of any of embodiments 20-21, which is fused to a heterologous polypeptide moiety directly or via a spacer, e.g. a spacer having a chain length of 1-50 amino acids, particularly selected from (i) Gly, Ser, Glu and/or Asp, or from (ii) Gly, Glu, Ser and Pro.
23. The VHH antibody of any one of embodiments 1-22, which is non-glycosylated.
24. The VHH antibody of any one of embodiments 1-23, which is produced in a bacterium, e.g. E. coli.
25. The VHH antibody of any one of embodiments 1-24, which is produced in a yeast, e.g. Pichia pastoris.
26. The VHH antibody of any of embodiments 1-25, which is conjugated to one or several polymer moieties, preferably hydrophilic polymer moieties, such as polyethylene glycol (PEG).
27. A set of two or more different VHH antibodies of any of embodiments 1-26.
28. The set of embodiment 27, wherein the different VHH antibodies recognize different epitopes on the RBD, particularly non-overlapping epitopes on the RBD.
29. The VHH antibody of any one of embodiments 1-26 or the set of embodiment 27 or 28 for use in medicine, e.g. human medicine, particularly for use in therapy or diagnostics.
30. The VHH antibody of any one of embodiments 1-26 or the set of embodiment 27 or 28 for use in the prevention or treatment of a disorder caused by and/or associated with an infection with SARS-CoV-2 including an infection with a SARS-CoV-2 escape mutant.
31. The VHH antibody of any one of embodiments 1-26 or the set of embodiment 27 or 28 for use in the prevention or treatment of Covid-19 in a human subject.
32. The VHH antibody of any one of embodiments 1-26 or the set of embodiment 27 or 28 for the detection of SARS-CoV-2 virus comprising
   (i) a set of at least two VHH antibodies recognizing different, particularly non-overlapping epitopes on the RBD,
   (ii) a set of at least two VHH antibodies comprising a set of capturing antibodies conjugated to a capturing moiety and a set of labeling antibodies conjugated to a labeling moiety, and/or
   (iii) a set of at least two VHH antibodies comprising a first set of labeling antibodies conjugated to a first labeling moiety, and a second set of labeling antibodies conjugated to a second labeling moiety, wherein the first labeling moiety is different from the second labeling moiety, wherein the first and the second labeling moieties are particularly selected from two spectrally different fluorescence labeling moieties.
33. Use of the VHH antibody of any one of embodiments 1-26 or the set of embodiment 27 or 28 for detecting SARS-CoV-2 virus or viral components in a patient sample, e.g. in a body fluid or tissue sample.
34. Use of the VHH antibody of any one of embodiments 1-26 or the set of embodiment 27 or 28 for detecting SARS-CoV-2 virus or viral components in a virus culture, e.g. in a culture of SARS-CoV-2 or variants thereof including pseudo typed variants, or in a genetically modified organism, e.g. in an organism producing viruses or viral components.
35. The use of embodiment 34 for monitoring, quantification and/or quality control during production of viruses or viral components.
36. A nucleic acid molecule encoding a VHH antibody according to any one of embodiments 1-26, preferably in operative linkage with a heterologous expression control sequence.
37. A vector comprising a nucleic acid molecule according to embodiment 36.
38. A recombinant cell or non-human organism transformed or transfected with a nucleic acid molecule according to embodiment 36 or a vector according to embodiment 37.
39. The cell or organism of embodiment 38, which is selected from a bacterium such as E. coli Bacillus sp., a unicellular eukaryotic organism, e.g. yeast such as Pichia pastoris, or Leishmania, an insect cell, a mammalian cell or a plant cell.
40. A method for recombinant production of a VHH antibody of any one of embodiments 1-26, comprising cultivating a cell or an organism of embodiment 38 or 39 in a suitable medium and obtaining the VHH antibody from the cell or organism or from the medium.
41. The method of embodiment 40, comprising cultivating a yeast such as Pichia pastoris and obtaining the VHH antibody from the medium.
42. A method for the prevention or treatment of a disorder caused by and/or associated with an infection with SARS-CoV-2, comprising administering an effective dose of the VHH antibody of any one of embodiments 1-26 or the set of embodiment 27 or 28 to a subject in need thereof, particularly to a human subject.
43. A homotrimeric VHH antibody comprising 3 VHH antibody subunits recognizing the same target structure, wherein each VHH antibody subunit comprises a VHH antibody directly linked or linked via a spacer to a trimerization module.
44. The homotrimeric VHH antibody of embodiment 43, which comprises three identical VHH antibody subunits.
45. The homotrimeric VHH antibody of embodiment 43 or 44 wherein the VHH antibody is directed to a target structure having a threefold-rotationally symmetric subunit structure.
46. The homotrimeric VHH antibody of any one of embodiments 43-45, wherein the VHH antibody is directed to a target structure comprising a homotrimeric protein or protein assembly.
47. The homotrimeric VHH antibody of any one of embodiments 43-46, wherein the target structure is a viral protein or protein assembly, particularly a homo-trimeric viral protein or protein assembly, more particularly selected from a Coronavirus trimeric spike protein, an Orthomyxovirus trimeric HA protein, a Paramyxovirus trimeric fusion protein, a Dengue fever virus or Zikavirus trimeric or higher order protein assembly, a Herpesvirus trimeric gB fusion protein or a HIV trimeric gp120 protein.
48. The homotrimeric VHH antibody of any one of embodiments 43-47, wherein the target structure is the SARS-CoV-2 spike protein S1 domain, particularly the receptor-binding domain (RBD) of the SARS-CoV-2 S1 domain including the RBD of wildtype SARS-CoV-2 and the RBD domain of a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.
49. The homotrimeric VHH antibody of embodiment 48, wherein the VHH antibody is selected from the VHH antibodies according to any one of embodiments 1-16.
50. The homotrimeric VHH antibody of embodiment 49, wherein the VHH antibody is selected from:
   (a) the VHH antibody VHH-72 comprising an VHH amino acid sequence as shown in SEQ. ID NO: 217, or
   (b) a sequence which has an identity of at least 70%, at least 80%, at least 90%, at least 95% or at least 99% to a VHH sequence of (a).
51. The homotrimeric VHH antibody of any one of embodiments 49-50, wherein the target structure is different from spike protein S1 domain, particularly from the receptor-binding domain (RBD) of the SARS-CoV-2 S1 domain.
52. The homotrimeric VHH antibody of any one of embodiments 49-51, wherein the target structure is a non-viral protein or protein assembly, particularly a member of the TNF Ligand superfamily or a member of the TNF Receptor superfamily.
53. The homotrimeric VHH antibody of any one of embodiments 49-52, wherein each subunit comprises a VHH antibody linked via a spacer to a trimerization module.
54. The homotrimeric VHH antibody of embodiment 53, wherein the spacer has a length of about 5 to about 100 amino acids, particularly of about 10 to about 50 amino acids.
55. The homotrimeric VHH antibody of embodiment 53 or 54, wherein the spacer comprises amino acids selected from Gly, Ser, Ala, Thr, Pro, Asp and Glu, particularly in an amount of at least about 50%, about 60%, about 70%, about 80%, about 90% or 100%.
56. The homotrimeric VHH antibody of embodiment 53 or 54, wherein the spacer comprises amino acids selected from Gly, Glu, Ser and Pro particularly in an amount of at least about 50%, about 60%, about 70%, about 80%, about 90% or 100%.
57. The homotrimeric VHH antibody of any one of embodiments 53-56, wherein the spacer comprises a succession of tripeptides, wherein the first and the second amino acid are selected from Gly, Ser, Ala, Thr, and Pro, and the third amino acid is selected from Asp and Glu.
58. The homotrimeric VHH antibody of any one of embodiments 53-57, wherein the spacer is substantially free or free of amino acids selected from Asn, Lys, Arg, Trp, Tyr, Phe, Met, Leu, Ile, Val, His and Cys.
59. The homotrimeric VHH antibody of any one of embodiments 43-58, wherein the trimerization module is selected from:
   (a) a collagen trimerization domain, e.g. the NC1 domain of collagen XV, the NC1 domain of collagen XVIII or the NC1 domain of collagen X, or the lung surfactant protein D,
   (b) a trimerization domain which has an identity of at least 80%, particularly at least 90%, more particularly at least 95% to a trimerization domain of (a).
60. A subunit of a homotrimeric VHH antibody of any one of embodiments 43-59.
61. A nucleic acid molecule encoding a homotrimeric VHH antibody according to any one of embodiments 43-59 or a subunit according to embodiment 60, preferably in operative linkage with a heterologous expression control sequence.
62. A vector comprising a nucleic acid molecule according to embodiment 61.
63. A recombinant cell or non-human organism transformed or transfected with a nucleic acid molecule according to embodiment 61 or a vector according to claim 62.
64. The cell or organism of embodiment 63, which is selected from a bacterium such as E. coli, Bacillus sp., a unicellular eukaryotic organism, e.g. yeast such as Pichia pastoris, Saccharomyces cerevisiae, or Hansenula polymorpha, or Leishmania, an insect cell, a mammalian cell or a plant cell.
65. The homotrimeric VHH antibody of any one of embodiments 43-59 or the subunit of embodiment 60, which is non-glycosylated.
66. The homotrimeric VHH antibody of any one of embodiments 43-59 or the subunit of embodiment 60, which is produced in a bacterium, e.g. E. coli.
67. The homotrimeric VHH antibody of any one of embodiments 43-59 or the subunit of embodiment 60, which is produced in a yeast, e.g. Pichia pastoris.
68. The homotrimeric VHH antibody of any one of embodiments 43-59 or 65-67 or the subunit of embodiment 60 or 65-67, which is conjugated to one or several polymer moieties, preferably hydrophilic polymer moieties, such as polyethylene glycol (PEG).
69. The homotrimeric VHH antibody of any one of embodiments 43-59 or 65-68 for use in medicine, e.g. human medicine, particularly for use in therapy or diagnostics, more particularly for use in the prevention or treatment of a disorder caused by and/or associated with an infection with SARS-CoV-2 including an infection with a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.
70. A method for recombinant production of a homotrimeric VHH antibody of any one of embodiments 43-59 or 65-68, comprising cultivating a cell or an organism of embodiment 63 or 64 in a suitable culture medium and obtaining the homotrimeric VHH antibody from the cell or organism or from the culture medium.
71. The method of embodiment 70, comprising cultivating a yeast such as Pichia pastoris Saccharomyces cerevisiae, or Hansenula polymorpha, and obtaining the homotrimeric VHH antibody from the medium.
72. A method for the recombinant production of a monomeric or multimeric VHH antibody, comprising:
   cultivating a yeast such as Pichia pastoris, Saccharomyces cerevisiae, or Hansenula polymorpha, comprising a nucleic acid molecule encoding the VHH antibody or a subunit thereof, in a suitable culture medium and
   obtaining the monomeric or multimeric, e.g. homotrimeric VHH antibody from the culture medium,
   wherein the monomeric or multimeric VHH antibody is particularly selected from a monomeric VHH antibody, a heterodimeric VHH antibody, or a homomultimeric VHH antibody, particularly a homotrimeric VHH antibody, more particularly a homotrimeric VHH antibody of any one of embodiments 43-59, 65 or 67.
73. The method of embodiment 72, wherein the yeast comprises a nucleic acid molecule encoding a polypeptide comprising (from N-terminus to C-terminus) (i) a cleavable co-translational signal sequence (pre-sequence), (ii) optionally a cleavable pro-sequence, which enhances secretion, and (iii) a sequence encoding a VHH antibody, e.g., a monomeric VHH antibody, a heterodimeric VHH antibody or a subunit of the homomultimeric, e.g. homotrimeric VHH antibody, particularly a fusion comprising the VHH antibody sequence, optionally a spacer, and a trimerization domain, e.g. the Collagen XVIII NC1 domain.
74. The method of embodiment 73, wherein the cleavable co-translational signal sequence (i) is selected from the Ost1 signal sequence of Saccharomyces cerevisiae (SEQ: ID NO. 219) or an Ost1 signal sequence from a related yeast species, e.g. from Pichia pastoris (SEQ: ID NO. 220), Schizosaccharomyces pombe (SEQ: ID NO. 221), or Candida albicans (SEQ: ID NO. 222) or a variant of those, e.g. having an amino acid identity of at least 80%, of at least 90% or of at least 95% thereto.
75. The method of embodiment 73 or 74, wherein the cleavable pro-sequence comprising a secretion signal sequence (ii) is selected from the propeptide of S. cerevisiae alpha-factor (SEQ: ID NO. 223) or a variant thereof, having an amino acid identity of at least 80%, of at least 90% or of at least 95%, or another Kex2-cleavable pro-peptide that promotes packaging into COPII vesicles and thus export from the ER.
76. A nucleic acid molecule encoding a polypeptide comprising (from N-terminus to C-terminus) (i) a cleavable co-translational signal sequence (pre-sequence), (ii) optionally a cleavable pro-sequence, which enhances secretion, and (iii) the sequence of a VHH antibody, e.g., a monomeric VHH antibody, a heterodimeric VHH antibody or a subunit of a homomultimeric, e.g. homotrimeric VHH antibody, particularly a fusion comprising the VHH antibody sequence, optionally a spacer, and a trimerization domain, e.g. the Collagen XVIII NC1 domain.
77. A polypeptide encoded by the nucleic acid molecule of embodiment 76, which comprises (from N-terminus to C-terminus) (i) a cleavable co-translational signal sequence (pre-sequence), (ii) optionally a cleavable pro-sequence, which enhances secretion, and (iii) a VHH antibody sequence, e.g., a monomeric VHH antibody, a heterodimeric VHH antibody or a subunit of the homomultimeric, e.g. homotrimeric VHH antibody, particularly a fusion comprising the VHH antibody sequence, optionally a spacer, and a trimerization domain, e.g. the Collagen XVIII NC1 domain.
78. A heterodimeric VHH antibody, particularly a covalently linked VHH heterodimer comprising a first VHH antibody and a second VHH antibody wherein the first VHH antibody and the second VHH antibody bind to different epitopes on the RBD of the SARS-CoV-2 S1 domain.
79. The heterodimeric VHH antibody of embodiment 78, which comprises two VHH antibodies that bind to non-overlapping RBD epitopes.
80. The heterodimeric VHH antibody of embodiment 78 or 79, wherein at least one VHH antibody is selected from the VHH antibodies according to any one of embodiments 1-17.
81. The heterodimeric VHH antibody of any one of embodiments 78-80, wherein the first and the second VHH antibody are fused through a linker sequence.
82. The heterodimeric VHH antibody of any one of embodiments 78-81, wherein the linker sequence is a peptidic linker, particularly having a length of 5 to 50 amino acid residues, particularly 10 to 40 amino acid residues and more particularly 12 to 30 amino acid residues.
83. The heterodimeric VHH antibody of embodiment 82, wherein the peptidic linker comprises amino acid residues selected from amino acid residues selected from Gly, Glu and Ser particularly in an amount of at least about 50%, at least about 60%, at least about 70%, at least about 80%, at least about 90% or 100%.
84. The heterodimeric VHH antibody of any one of embodiments 78-83, wherein the first VHH antibody competes with a VHH antibody selected from Re6B07, Re7E02 or Re9F06, particularly with VHH antibody Re9F06.
85. The heterodimeric VHH antibody of any one of embodiments 78-84, wherein the first VHH antibody is selected from Re9F06 or a variant thereof, or from Re5F10 or a variant thereof.
86. The heterodimeric VHH antibody of any one of embodiments 78-85, wherein the second VHH antibody competes with a VHH antibody selected from Re5D06, Re6B06, Re6D06, Re6H06, Re9B09, Re9C08, or Re9H01, particularly with VHH antibody Re5D06.
87. The heterodimeric VHH antibody of any one of embodiments 78-86, wherein the second VHH antibody is selected from Re5D06 or a variant thereof, more particularly from a thermostable or hyperthermostable variant thereof such as Re5D06R28.
88. The heterodimeric VHH antibody of any one of embodiments 78-87, wherein the first VHH antibody is selected from a VHH antibody comprising a VHH sequence as shown in SEQ. ID NO 29 (Re5F10), 248 (Re9F06), 268 (Re22D04), 276 (Re25H10), 260 (Re21D01), 284 (Re26E09) and 288 (Re26E11) or a VHH antibody, which is a variant thereof, and/or wherein the second VHH antibody is selected from a VHH antibody comprising a VHH sequence as shown in SEQ. ID NO 17 (Re5D06), or a VHH antibody, which is a variant thereof, e.g. SEQ. ID NO: 232 (Re5D06R28D), SEQ. ID NO: 240 (Re5D06R15_3QE) or SEQ. ID NO: 244 (Re5D06R28_3QE), or as shown in SEQ. ID NO: 81 (Re6H06), SEQ. ID NO: 129 (Re9B09), SEQ. ID NO: 272 (Re22E05) or SEQ. ID NO: 280 (Re26D07) or a VHH antibody, which is a variant thereof.
89. The heterodimeric VHH antibody of any one of embodiments 78-88, which is non-glycosylated.
90. The heterodimeric VHH antibody of any one of embodiments 78-89, which is produced in a bacterium, e.g. E. coli.
91. The heterodimeric VHH antibody of any one of embodiments 78-90, which is produced in a yeast, e.g. Pichia pastoris.
92. The heterodimeric VHH antibody of any one of embodiments 78-91 for use in medicine, e.g. human medicine, particularly for use in therapy or diagnostics.
93. The heterodimeric VHH antibody of any one of embodiments 78-92 for use in the prevention or treatment of a disorder caused by and/or associated with an infection with SARS-CoV-2 including an infection with a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.
94. The heterodimeric VHH antibody of any one of embodiments 78-91 for use in the prevention or treatment of Covid-19 in a human subject.
95. A nucleic acid molecule encoding a heterodimeric VHH antibody according to any one of embodiments 78-91, preferably in operative linkage with a heterologous expression control sequence.
96. A vector comprising a nucleic acid molecule according to embodiment 95.
97. A recombinant cell or non-human organism transformed or transfected with a nucleic acid molecule according to embodiment 94 or a vector according to embodiment 96.
98. The cell or organism of embodiment 97, which is selected from a bacterium such as E. coli Bacillus sp., a unicellular eukaryotic organism, e.g. yeast such as Pichia pastoris, or Leishmania, an insect cell, a mammalian cell or a plant cell.
99. A method for recombinant production of a heterodimeric VHH antibody of any one of embodiments 78-91, comprising cultivating a cell or an organism of embodiment 97 or 98 in a suitable medium and obtaining the VHH antibody from the cell or organism or from the medium.
100. The method of embodiment 99, comprising cultivating a yeast such as Pichia pastoris and obtaining the VHH antibody from the medium.
101. A method for the prevention or treatment of a disorder caused by and/or associated with an infection with SARS-CoV-2, comprising administering an effective dose of the heterodimeric VHH antibody of any one of embodiments 78-91 to a subject in need thereof, particularly to a human subject.

### DETAILED DESCRIPTION OF THE INVENTION

According to the present invention, SARS-CoV-2-neutralizing VHH antibodies are provided, which are suitable for the effective treatment of COVID-19 patients. Specifically, the invention provides VHH antibodies that neutralize virus already at a very low concentration. Further, the invention provides stable VHH antibodies.

Furthermore, the invention provides multimeric, particularly trimeric VHH antibodies for enhancing the potency of VHH antibodies, which may be directed against SARS-CoV-2 or against other target structures, e.g. for blocking infections by other viruses.

Furthermore, the invention provides heterodimeric VHH antibodies comprising a first VHH antibody and a second VHH antibody wherein the first VHH antibody and the second VHH antibody bind to different epitopes on the RBD of the SARS-CoV-2 S1 domain.

SARS-CoV-2-Neutralising VHH Antibodies

The primary aim of this aspect of the invention has been to generate, select, engineer and optimize SARS-CoV-2-neutralising VHH antibodies for effective treatment of COVID-19 patients. Specifically, we aimed for VHH antibodies that are capable of virus neutralization already at a very low concentration. Further, we aimed for VHH antibodies that have sufficient stability for pharmaceutical applications.

The present invention relates to a VHH antibody, which is a monovalent heavy chain-only antibody comprising a CDR1 domain, a CDR2 domain and a CDR3 domain linked by framework regions including, but not being limited to, whole VHH antibodies, e.g. native VHH antibodies comprising framework regions derived from camelids, and modified VHH antibodies comprising modified framework regions, VHH antibody fragments and VHH antibody fusion proteins, e.g. a fusion protein with an immunoglobulin or non-immunoglobulin peptide or polypeptide, as long as it shows the properties according to the invention.

The present invention is also directed to a covalent or non-covalent conjugate of a VHH antibody molecule to a non-proteinaceous structure, for example, a labeling group, a capture group such a solid phase-binding group, or an effector group such as a toxin. For example, the heterologous moiety may be from a fluorescence group, biotin, an enzyme such as a peroxidase, phosphatase or luciferase, a hapten, an affinity tag, or a nucleic acid such as an oligonucleotide.

The VHH antibody of the present invention is particularly a monoclonal VHH antibody characterized by a specific amino acid sequence. The VHH antibody may be produced in a prokaryotic host cell, a yeast cell or a mammalian cell. In certain embodiments, the VHH antibody is non-glycosylated. In certain embodiments, the VHH antibody is glycosylated, wherein a carbohydrate structure may be derived from a glycosylation site introduced into the VHH sequence and/or from a fusion partner.

A VHH antibody according to the present invention is characterized by (i) a CDR3 sequence, (ii) a combination of a CDR1 sequence, a CDR2 sequence, and a CDR3 sequence, (iii) by a complete VHH sequence, or (iv) by competition with a specific reference antibody. Specific CDR and VHH sequences are provided in the Tables, Figures and the Sequence Listing. According to the present invention, sequences related to the above sequences are encompassed. These related sequences are defined by having a minimum identity to a specifically indicated amino acid sequence, e.g. a CDR or VHH sequence. This identity is indicated over the whole length of the respective reference sequence and may be determined by using well known algorithms such as BLAST.

In particular embodiments, a related CDR3 sequence has an identity of at least 80% or at least 90% or at least 95% to a specifically indicated CDR3 sequence, e.g. a substitution of 1, 2, or 3 amino acids.

In particular embodiments, a related combination of a CDR1 sequence, a CDR2 sequence, and a CDR3 sequence has an identity of at least 80% or at least 90% or at least 95% to a specifically indicated combination of a CDR1 sequence, a CDR2 sequence, and a CDR3 sequence, e.g. a substitution of 1, 2, 3, 4, 5 or 6 amino acids by different amino acids.

In particular embodiments, a related VHH sequence has an identity of least 70%, at least 80%, at least 90%, at least 95% or at least 99% to a VHH sequence, e.g. a substitution of 1, 2, 3, 4, 5 or up to 20 amino acids.

Further, the invention refers to a VHH antibody, which competes with a specific VHH antibody disclosed herein for the binding to the SARS-CoV-2 spike protein S1 domain. In certain embodiments, a competing VHH antibody binds the same or an overlapping epitope on the SARS-CoV-2 spike protein S1 domain, particularly the receptor-binding domain (RBD) of the SARS-CoV-2 S1 domain as a specific VHH antibody disclosed herein. For example, the invention refers to a VHH antibody, which competes either with the VHH antibodies Re5D06, Re6B06, Re6D06, Re6H06, Re9B09, Re9C08, or Re9H01, or with VHH antibodies Re6B07 or Re7E02.

In certain embodiments, the invention relates to a VHH antibody competing with VHH antibody Re5D06, such as any one of the VHH antibodies Re6B02, Re7B01, Re10B10, or Re10F10. In certain embodiments, the invention relates to a VHH antibody competing with VHH antibody Re7E02, such as the VHH antibody Re6B07.

Competition is measured either as an at least 90% loss of fluorescence staining of the SARS-CoV-2 spike protein by a fluorophore-labelled candidate VHH antibody in the presence of a 100-fold molar excess of an unlabelled competitor VHH antibody e.g. selected from Re5D06, Re6B06, Re6D06, Re6H06, Re9B09, Re9C08, Re9H01, or Re7E02, or conversely as an at least 90% loss of spike protein-staining by a fluorophore-labelled VHH antibody e.g. selected from Re5D06, Re6B06, Re6D06, Re6H06, Re9B09, Re9C08, Re9H01, or Re7E02 in the presence of a 100-fold molar excess of the unlabelled candidate VHH antibody as a competitor.

Further, the invention refers to a VHH antibody, which competes with a specific VHH antibody disclosed herein for the binding to the SARS-CoV-2 spike protein S1 domain. In certain embodiments, a competing VHH antibody binds the same or an overlapping epitope on the SARS-CoV-2 spike protein S1 domain, particularly the receptor-binding domain (RBD) of the SARS-CoV-2 S1 domain as a specific VHH antibody disclosed herein. For example, the invention refers to a VHH antibody, which competes either with the VHH antibodies Re5D06 and/or Re6D06 or with VHH antibodies Re6B07 and/or Re7E02.

In particular embodiments, at least one amino acid of a reference sequence, including an amino acid in a CDR1, CDR2 or CDR3 sequence and/or an amino acid in a framework region, is replaced by another amino acid, while preserving structural integrity and epitope-binding of the VHH antibody. These exchanges can be conservative (i.e., by a similar amino acid) or non-conservative.

In further particular embodiments, at least one amino acid of a reference sequence, including an amino acid in a CDR1, CDR2 or CDR3 sequence and/or an amino acid in a framework region, is replaced by a conservative amino acid substitution, i.e. a substitution of an amino acid by another amino acid with similar biochemical properties, for example a substitution of an aliphatic amino acid, e.g. Gly, Ala, Val, Leu, or Ile, for another aliphatic amino acid; a substitution of a basic amino acid, e.g. His, Lys or Arg, against another basic amino acid or against Met; a substitution of an acidic amino acid or an amide thereof, e.g. Asp, Glu, Asn or Gln, against another acidic amino acid or an amide thereof; a substitution of an aromatic amino acid, e.g. Phe, Tyr or Trp, against another aromatic amino acid.

In further particular embodiments, the amino acid Ser, particularly the amino acid Ser11 of the CDR3 sequence of the VHH antibody Re5D06 (SEQ: ID NO. 20) is replaced by a different amino acid, e.g. Glu in Re5D06R13 (SEQ: ID NO 205).

In further particular embodiments, the VHH antibody comprises the VHH sequence of VHH antibody Re5D06 (SEQ: ID NO. 17) or a variant thereof. For example, the VHH antibody comprises an amino acid sequence wherein at least one amino acid of the VHH sequence of VHH antibody Re5D06 (SEQ: ID NO. 17) including an amino acid in a CDR1, CDR2 or CDR3 sequence and/or an amino acid in a framework region, is replaced by another amino acid, for example at least one of the amino acid substitutions A261, 129S, 136M, Q41E, S51A, 153W, S55N, S56N, T60V, N61D, N79D, V81Y, K89E, V95D, Y106X1 (with X1 being an amino acid residue selected from D, N, or E), S108X2 (with X2 being any amino acid residue except for C), S109X3 (with X3 being any amino acid residue, in particular E, Q or K, except for C or P), and Y111H. In certain embodiments, 1, 2, 3, 4, 5, 6, 7, 8, 9 or 10 amino acids of SEQ ID NO: 17 are replaced by another amino acid. In even more particular embodiments, the VHH sequence is as shown in SEQ: ID NO. 201 (Re5D06R11), 205 (Re5D06R13), 224 (Re5D06R15), 228 (Re5D06R23), 232 (Re5D06R28), 236 (Re5D06R28D), 240 (Re5D06R15_3QE), or 244 (Re5D06R28_3QE).

In further particular embodiments, the VHH antibody is selected from antibody Re25H10 comprising a VHH sequence as shown in SEQ: ID NO. 276 or a VHH antibody, which is a variant thereof. In certain embodiments, 1, 2, 3, 4, 5, 6, 7, 8, 9 or 10 amino acids of SEQ ID NO: 276 are replaced by another amino acid.

In further particular embodiments, the VHH antibody is selected from antibody Re6H06 comprising a VHH sequence as shown in SEQ: ID NO. 73 or a VHH antibody, which is a variant thereof, particularly a variant as shown in SEQ: ID NO. 280 (Re26D07). In certain embodiments, 1, 2, 3, 4, 5, 6, 7, 8, 9 or 10 amino acids of SEQ ID NO: 73 are replaced by another amino acid.

In further particular embodiments, the VHH antibody is selected from antibody Re9H03 comprising a CDR3 sequence as shown in SEQ: ID No. 252 or a VHH antibody, which is a variant thereof, particularly a variant as shown in SEQ: ID NO. 272 (Re22E05). In certain embodiments, 1, 2, 3, 4, 5, 6, 7, 8, 9 or 10 amino acids of SEQ ID NO: 252 are replaced by another amino acid.

In further particular embodiments, the VHH antibody is selected from antibody Re5F10 comprising a VHH sequence as shown in SEQ: ID NO. 29 or a VHH antibody, which is a variant thereof, particularly a variant as shown in SEQ: ID NO. 260 (Re21 D01), 284 (Re26E09), or 288 (Re26E11). In certain embodiments, 1, 2, 3, 4, 5, 6, 7, 8, 9 or 10 amino acids of SEQ ID NO: 29 are replaced by another amino acid.

In further particular embodiments, the VHH antibody is selected from antibody Re21H01 comprising a CDR3 sequence as shown in SEQ: ID NO. 264 or a VHH antibody, which is a variant thereof. In certain embodiments, 1, 2, 3, 4, 5, 6, 7, 8, 9 or 10 amino acids of SEQ ID NO: 264 are replaced by another amino acid.

The VHH antibody of the present invention is binding to the S1 domain, particularly to the RBD of the S1 domain of SARS-CoV-2. In certain embodiments, a VHH antibody is capable of virus neutralization at a concentration of about 10 nM or less, of about 1 nM or less, of about 500 pM or less, of about 100 pM or less or even at about 50 pM or less.

In certain embodiments, a VHH antibody is capable of neutralizing a SARS-CoV2 mutant, in particular a SARS-CoV-2 escape mutant. In certain further embodiments, the VHH antibody is capable of neutralizing a SARS-CoV2 mutant comprising a spike protein RBD including at least one amino acid substitution in the RBD selected from the group consisting of K417T, K417N, L452R, E484K, and N501Y. Thus, the present invention provides a VHH antibody capable of neutralizing a SARS-CoV-2 escape mutant, e.g. the UK mutant (Alpha variant, B.1.1.7), the South African mutant (Beta variant, B.1.351), the Brazilian mutant (Gamma variant, P.1), the Indian mutant (Delta variant, B.1.617) and/or the Californian mutant (Epsilon variant, B.1.427).

Further, the present invention relates to a nucleic acid molecule, e.g. a DNA molecule, encoding a VHH as indicated above, or a vector, comprising said nucleic acid molecule as indicated above in operative linkage with an expression control sequence, particularly with a heterologous expression control sequence. Furthermore, the invention relates to a cell comprising a nucleic acid molecule or a vector as described above. Vectors for the recombinant production of VHH antibodies are well known in the art. In certain embodiments, the vector is an extrachromosomal vector. In other embodiments, the vector is a vector for genomic integration. The cell may be a known host cell for producing antibodies or antibody fragments, e.g. a prokaryotic cell such as an E. coli or a Bacillus sp. cell, a yeast cell, particularly a Pichia yeast cell, an insect cell or a mammalian cell, e.g. a CHO cell, or a plant cell. In certain embodiments, the cell comprises the nucleic acid or the vector extrachromosomally. In other embodiments, the cell comprises the nucleic acid or the vector integrated into the genome, e.g. as a genomically integrated expression cassette.

Still a further aspect of the present invention is a method of recombinantly producing a VHH antibody by growing a cell as described above in a culture medium and obtaining the VHH antibody from the cell or the culture medium. Suitable culture media and culture conditions are well known in the art.

Sets of VHH Antibodies

In a further aspect, the present invention relates to a set comprising at least 2, 3, 4 or more of the above VHH antibodies. In such a set, the individual VHH antibodies are present in suitable molar ratios. Typically, the molar ratios are in the range of about 2:1 to about 1:2, particularly about 1.5:1 to about 1:1.5, even more particularly about 1:1. In certain embodiments, the VHH antibody set may comprise a single composition wherein the VHH antibodies in said set consist of a predetermined number of different species of VHH antibodies as described above. The VHH antibody set may comprise a plurality of compositions each comprising a different species of VHH antibody as described above. The set of the present invention may be free from other VHH antibodies.

The inventors' target structure has been the S1 fragment of the SARS-CoV-2 spike protein (Wrapp et al., 2020), which contains two main globular domains: A large N-terminal, lectin-like domain (residues 27-290) as well as a receptor-binding domain (RBD, residues 330-521). The RBD mediates the interaction with the ACE2 host cell receptor and is thus essential for infectivity.

The inventors applied five subcutaneous immunizations in weekly intervals such that the S1 fragment and in particular the RBD were presented in multiple contexts to the animals' immune systems. As antigens were used: transfected HEK293 cells expressing the complete spike protein on its surface (10⁷ cells per immunization) and up to 100 μg of the following purified proteins: fusions of the RBD and the entire S1 domain to mouse and human Fc fragments as well as an RBD fusion to the E. coli DsbG dimer. Four days after the last immunization, blood samples were collected, RNA from ˜10⁸ lymphocytes each was isolated, VHH-coding regions were amplified by nested RT-PCRs, cloned as cDNAs into a phagemid to yield three separate immune libraries with complexities of around 10⁹ independent clones each. These libraries were subjected to phage display, using as baits either an immobilized RBD (yielding anti-RBD VHHs) or an immobilized S1 fragment with competition by an excess of free RBD (to yield anti-S1ΔRBD VHH antibodies). Bovine Serum Albumin, as well as human and murine serum, were included as non-specific competitors.

A total of 672 selected clones were so far sequenced and classified according to sequence similarity. Representatives of all VHH classes were then expressed in E. coli and purified (see FIG. 1 for specific sequences of VHHs). One set of nanobodies was expressed with ectopic cysteines for labelling with fluorophore-maleimides (Pleiner et al., 2015). These labelled nanobodies were used for immunofluorescence on HeLa cells that had been transfected with a SARS CoV2 spike expression construct. Because only a fraction of cells gets transfected, a typical field of view contains non-transfected cells (serving as internal negative control) as well as transfected ones. Several of the labelled nanobodies stained the expressed spike protein very brightly. FIG. 2 shows VHH Re6D09 labelled with Alexa568 as an example. Table 1 (supra) lists the VHHs that have been tested positive for anti-Spike fluorescence.

In a second set of experiments, the inventors used cultured cells infected with SARS-CoV-2 isolated from a patient's sample. It was observed that numerous VHH antibodies (e.g. Re5A08, Re5B06, Re5D05, Re6D06, and Re6D09) stained viral structures brightly and specifically as judged by a lack of signal in non-infected cells (see FIG. 3 for example). Particularly, the VHH antibody Re6D06 and antibodies of the same class, e.g. Re9B10, gave the brightest stainings. Thus, VHH antibodies of this invention are suitable for detecting SARS-CoV-2 spike components in biological samples.

In the third set of experiments, the inventors performed tests if any of the obtained nanobodies can neutralize SARS-CoV-2 and prevent the virus from infecting a target cell. In these experiments, the Vero E6 cell line was used, which expresses high levels of the SARS-CoV-2 receptor ACE2 (Li et al., 2003) but is deficient in its interferon response (Emeny and Morgan, 1979); it is therefore extremely susceptible to viral infection. Moreover, a SARS-CoV-2 patient derived virus strain was used carrying the infection-enhancing D614G spike mutation (Zhang et al., 2020). This together therefore combines into a highly stringent test system for anti-viral inhibition. Without VHH antibodies, the addition of virus caused a pronounced cytopathic effect (CPE), and the amount of virus increased within three days of incubation approximately 10 000-fold.

When tested at a concentration of 500 nM (7.5 mg/liter), 43 anti-RBD VHHs were able to protect cells from the CPE and prevented virus replication completely (see Table 1 and FIG. 4 for examples). These VHH antibodies are now excellent candidates for treating SARS-CoV-2-infected patients.

Lowest VHH Antibody Concentration of Virus Neutralization

An important issue in pharmaceutical development is the lower limit of concentration at which an anti-viral antibody is still effective. Indeed, it makes a great difference if grams (as in the case of rabies post-exposure prophylaxis) or one milligram are required for an effective therapeutic dose. This determines the costs per treatment and, if many patients need to be treated, it matters how many therapeutic doses can be produced at all. Likewise, adverse side effects by the antibody itself will scale with the applied dose. Finally, contaminants, such as endotoxins, can cause side effects, and the absolute amount of contaminant will scale-up with the administered dose. It is thus highly desirable to obtain VHH antibodies that effectively block viral infection at the lowest possible concentration.

In particular embodiments, the present invention relates to a neutralizing VHH antibody, e.g. a VHH antibody which has a lowest SARS-CoV-2 neutralizing concentration of about 500 pM or less, of about 250 pM or less, of about 170 pM or less, of about 100 pM or less, of about 50 pM or less or of about 17 pM or less.

The inventors tested their collection of anti-RBD VHHs (Table 1) for neutralization potency. For that, SARS-CoV-2 was pre-incubated with VHH dilution series before adding the virus to Vero E6 cell cultures. 2-3 days later, infection (respectively block of infection) was scored by CPE, detection of newly produced viral components (using fluorescently labelled anti Spike VHHs) and quantitation of RNA replication by quantitative RT PCR. These readouts were used to determine the lowest VHH concentration that still reliably neutralized the virus completely (IC99+ values). Lowest SARS-CoV-2-neutralizing concentrations of selected VHH antibodies are listed that fully suppressed infection over a period of three days. Table 3 lists a selection of particularly potent SARS-CoV-2 neutralizers.

This identified several highly potent, SARS-CoV-2-neutralizing VHHs. Re9H01 and Re9B09 neutralized already at a concentration of 170 pM. Re5D06 and Re6H06 even at 50 pM. This corresponds to ˜0.65 μg VHH antibody per liter. When injected into a patient, and assuming that it gets diluted into an extracellular volume of 15 liters, then 100 μg should already be sufficient to exceed the therapeutically effective concentration in the extracellular space ten-fold.

Further it was found, that several VHH antibodies belong to a certain class of VHH antibodies, e.g. class Re5D06, class Re6B07 or Re6D06, i.e. they compete with the respective VHH antibody Re5D6, Re6B07 or Re6D06 for the binding to the RBD. Thus, the VHH antibodies of an individual class compete with each other, e.g. they recognize the same or an overlapping epitope on the RBD. Table 3 shows the neutralization potency and thermostability of selected anti-SARS-CoV-2 VHH antibodies.

Hyperthermostable SARS-CoV-2 Neutralizers

For the intended therapeutic application, the anti-SARS-CoV-2 VHH antibodies should not only be highly potent in virus neutralization, but also, they should be developable as biological drugs. This includes that they are stable enough to survive a lengthy, large-scale production process as well as transportation and storage (ideally for years in liquid formulation) without aggregation or loss of activity.

A good predictor for stability is thermostability, which can be measured, e.g., by thermal shift assays or specifically by differential scanning fluorimetry.

In a particular embodiment, the invention relates to a VHH antibody, which is stable, particularly thermostable or hyperthermostable. Preferably, the VHH antibody has a melting point (melting temperature) of at least about 65° C., of at least about 80° C., of at least 90° C. or of at least about 95° C., and/or an aggregation temperature of at least about 50° C., of at least about 60° C., of at least 70° C. or of at least about 80° C. when measured under non-reducing conditions and/or under reducing conditions. Melting and aggregation temperatures are determined as described herein.

Applying this assay to the super-neutralizing VHH antibody Re5D06 revealed a melting point of 50° C. when expressed cytoplasmically in the E. coli strain NEB Shuffle (FIG. 6). When expressed in Pichia pastoris and secreted from the host cell, an improved thermostability, i.e. a melting point of 65° C. was obtained, which can be explained by a more complete formation of the structural disulfide bond.

To further improve the properties of Re5D06, the inventors solved the structure of the Re5D06-RBD complex by X-ray crystallography and used the structural information to guide mutagenesis. The crystal structure of the RBD-Re5D06 complex revealed a potentially destabilizing cavity within the VHH antibody's hydrophobic core. This cavity hosted the hydrophobic portion of the used crystallization additive (benzyl dimethyl ammonium propane sulfonate). In turn, this suggested that the packing of the hydrophobic core could be improved and that this might stabilize the VHH antibody further.

Core II (136M, Q41E, S51A, N79D, V81Y, and K89E) was the first Re5D06-stabilizing design. It comprises two sets of mutations, a first set (136M, S51A, and V81Y) for stabilizing the hydrophobic core and a second set (Q41E, N79D, and K89E) for establishing additional, stabilizing salt bridges.

Core III (A261, 129S, 136M, Q41E, S51A, N79D, V81Y, and K89E) is based on Core II but contains A261 and 129S as additional stabilizing mutations.

Core IV (A261, 129S, 136M, Q41E, S51A, 153W, N79D, V81Y, and K89E) is based on Core III but contains an additional 153W mutation.

Starting from these core designs, the inventors tested a series of specific variants. A first variant of Re5D06, designated Re5D06R11 comprises the mutations (i) 136M, S51A, and V81Y, (ii) Q41E, N79D, and K89E, and (iii) S56N and S109E for stabilizing the conformation of CDR3 and shielding the epitope-distal side of its critical residue Y111 (numbering according to FIG. 1 and the sequence protocol).

A second variant Re5D06R13 comprises additional mutations T60V and V95D for improving side chain packing and polarity at the surface.

Remarkably, both Re5D06R11 and Re5D06R13 variants showed no signs of melting even at 95° C. and where highly resistant against heat-induced aggregation, while retaining a potency (IC99+ neutralization at 170 pM) close the Re5D06 original. They are thus in the category of the most thermostable antibodies/VHHs ever reported so far (for comparisons see: Hussack et al., 2011; Kunz et al., 2017).

A third variant Re5D06R15 combined the Core II mutations set with additional S55N and V95D exchanges. This variant was hyperthermostable to >95° C. as well. It showed an extraordinary anti-viral potency and achieved a complete SARS-CoV-2 neutralization (IC99+) down to a monomer concentration of 17 pM or 0.2 μg per liter.

A fourth variant Re5D06R23 is based on the Core III design and carries the additional S55N, S56N, N61 D, and V95D mutations. It was also stable to 95° C. and neutralized down to 170 pM.

A fifth variant Re5D06R28 is based on Core IV and comprises the additional S55N, S56N, V95D, as well as an Y111H exchange in CDR3. Y111 in the original Re5D06 stacks to F490 of the RBD. A histidine in this position retains this stacking. In addition, it forms a salt bridge to E484 of the RBD, resulting in a faster on-rate of RBD-binding. Accordingly, this variant neutralizes down to a concentration of 17 pM.

In addition, the inventors identified several VHH antibodies that were hyperthermostable even before any optimization (Table 3).

TABLE 3

| | | Expression | Lowest SARS-COV-2- | | | | | |
|---|---|---|---|---|---|---|---|---|
| VHH | Class | System | neutralizing concentration | | T_{Melt} | | T_{Aggr} | |
| Re5D06 | Re5D06 | E. coli Shuffle/cytoplasmic | 50 | pM | 50° | C. | 50° | C. |
| Re5D06 | Re5D06 | Secretion Pichia | 50 | pM | 65° | C. | 53° | C. |
| Re5D06R11 | Re5D06 | E. coli Shuffle/cytoplasmic | 170 | pM | >95° | C. | >80° | C. |
| Re5D06R13 | Re5D06 | E. coli Shuffle/cytoplasmic | 170 | pM | >95° | C. | >80° | C. |
| Re5D06R15 | Re5D06 | E. coli Shuffle/cytoplasmic | 17-50 | pM | >95° | C. | >80° | C. |
| Re5D06R23 | Re5D06 | E. coli Shuffle/cytoplasmic | 170 | pM | >95° | C. | >80° | C. |
| Re5D06R28 | Re5D06 | E. coli Shuffle/cytoplasmic | 17-50 | pM | >95° | C. | >80° | C. |
| Re9F06 | Re9F06 | E. coli Shuffle/cytoplasmic | 50 | nM | >95° | C. | | |
| Re9H01 | Re9H01 | E. coli Shuffle/cytoplasmic | 170 | pM | 37° | C. | 35° | C. |
| Re9B09 | Re9B09 | Secretion Pichia | 170 | pM | 64° | C. | 66° | C. |
| Re9H03 | Re9B09 | E. coli Shuffle/cytoplasmic | 170 | pM | 60° | C. | | |
| Re5F10 | Re5F10 | E. coli periplasmic | 1.7 | nM | >95° | C. | | |
| Re21D01 | Re5F10 | E. coli periplasmic | 1.7 | nM | >95° | C. | | |
| Re26E09 | Re5F10 | E. coli periplasmic | 1.7 | nM | >95° | C. | | |
| Re21H01 | Re21H01 | E. coli periplasmic | 5 | nM | >95° | C. | | |
| Re25H10 | Re25H10 | E. coli Shuffle/cytoplasmic | | | >95° | C. | | |
| Re21B09 | Re21B09 | E. coli periplasmic | | | >95° | C. | | |
| Re6H06 | Re6H06 | E. coli periplasmic | 50 | pM | >95° | C. | >80° | C. |
| Re26D07 | Re6H06 | E. coli periplasmic | 50 | pM | >95° | C. | | |
| Re6B06 | Re6B06 | E. coli Shuffle/cytoplasmic | 170 | nM | >95° | C. | >80° | C. |
| Re7H02 | Re6B06 | E. coli Shuffle/cytoplasmic | 17 | nM | >95° | C. | >80° | C. |
| KG4B11 | Re6B06 | E. coli Shuffle/cytoplasmic | 17 | nM | >95° | C. | >80° | C. |

| | | | | | | |
|---|---|---|---|---|---|---|
| Re10B10 | Re5D06 | E. coli Shuffle/cytoplasmic | 50 | pM | n.d. | n.d. |
| Re6B02 | Re5D06 | E. coli Shuffle/cytoplasmic | 50 | pM | n.d. | n.d. |
| Re10F10 | Re5D06 | E. coli Shuffle/cytoplasmic | 50 | pM | n.d. | n.d. |
| Re7B01 | Re5D06 | E. coli Shuffle/cytoplasmic | 50 | pM | n.d. | n.d. |
| Re9B10 | Re6D06 | E. coli Shuffle/cytoplasmic | 500 | pM | n.d. | n.d. |
| Re6D06 | Re6D06 | E. coli Shuffle/cytoplasmic | 500 | pM | n.d. | n.d. |
| Re9C08 | Re8C08 | E. coli Shuffle/cytoplasmic | 500 | pM | n.d. | n.d. |
| Re7E02 | Re6B07 | E. coli Shuffle/cytoplasmic | 500 | pM | n.d. | n.d. |

Neutralization potency and thermostability of selected VHH antibodies. Melting temperatures were determined as described in FIG. 6. The onset of aggregation was determined by dynamic light scattering (DLS) using a Wyatt DynaPro machine, heating the sample @ 1 mg/ml protein concentration slowly (1°/min) up to the maximum temperature of 80° C. n.d .: not determined.

These include Re5F10 (and the related VHHs Re21D01, Re26E09, and Re26E11), Re9F06 (and the related Re6A11), Re2l H01, Re6B06 (and the related VHHs KG4B11 and Re7H02), Re6H06 (and the related VHH Re26D07), Re21B09, and Re25H10.

The VHH antibodies Re5F06, Re6H06, Re21 D01, and Re21H01 are stabilized by an additional disulfide bond.

The set of hyperthermostable VHHs includes some extremely strong RBD-binders and very potent neutralizers, such as Re6H06 (K_{D}≤1 pM, neutralization at 50 pM).

Neutralization of SARS-CoV-2 Escape Mutations

By now, several SARS-CoV-2 strains with mutated RBDs have emerged that are causing devastating outbreaks. These include the UK B.1.1.7 variant with an N501Y mutation in the RBD, the South African B.1.351 variant (K417N, E484K, N501Y), Brazilian P1 (K417T, E484K, N501Y), New York City B.1.526 (S477N, E484K), Californian B.1.429 (L452R) as well as the Indian B.1.617 strain (L452R, E484Q).

The emergence of such mutant strains is presumably driven by the selective pressure of the immune system, particularly in immunocompromised, persistently infected patients (Clark et al., 2021; Starr et al., 2021). Escape mutants can bypass antibody-based immunity in previously infected or vaccinated individuals. This has the potential of sustaining infection waves in populations that have acquired herd immunity against earlier strains.

Escape mutants also pose a tremendous challenge for therapeutic antibodies, which may be rendered ineffective by a given mutation. Indeed, several monoclonal anti-SARS-CoV-2 antibodies developed for treating COVID-19 are ineffective against the South African and Brazilian variants (Garcia-Beltran et al., 2021; Hoffmann et al., 2021; Li et al., 2021; Tada et al., 2021; Wibmer et al., 2021; Zhou et al., 2021).

Given the relevance of the now circulating RBD mutations, the inventors decided to explore their impact on their VHH leads. They found that Re5D06-binding to the RBD was not affected by the N501Y exchange. Thus, Re5D06 was found to be active against the British variant.

However, the combined South African (K417N, E484K, N501Y) or Brazilian (K417T, E484K, N501Y) mutations, weakened the Re5D06⋅RBD interaction to 0.1-0.5 nM K_{D}s. The Californian L452R mutation also reduced affinity to ˜400 pM.

Heterodimeric VHH Antibodies

A further aspect of the invention relates to a heterodimeric VHH antibody, particularly a covalently linked VHH heterodimer comprising a first VHH antibody and a second VHH antibody wherein the first VHH antibody and the second VHH antibody bind to different epitopes on the RBD of the SARS-CoV-2 S1 domain.

A heterodimeric VHH antibody comprises two different VHH antibodies optionally connected by linker. In particular embodiments, at least one of the VHH antibodies is a VHH antibody of the present invention as described herein. A VHH antibody of a heterodimeric VHH antibody is characterized by (i) a CDR3 sequence, (ii) a combination of a CDR1 sequence, a CDR2 sequence, and a CDR3 sequence, (iii) by a complete VHH sequence, or (iv) by competition with a specific reference antibody.

In order to provide VHH antibodies active against SARS-CoV-2 escape mutants including the South African, Brazilian and Indian variants, the inventors constructed a heterodimeric VHH antibody comprising a first VHH antibody and a second VHH antibody wherein the first VHH antibody and the second VHH antibody bind to different non-overlapping epitopes on the RBD of the SARS-CoV-2 S1 domain. In certain embodiments, the first and the second VHH antibody do not compete with each other as described above. The first and the second VHH antibody may be directly fused to each other or fused through a linker sequence. In particular embodiments, the first and the second VHH antibody are genetically fused by a peptidic linker sequence.

In certain embodiments, the linker is a peptidic linker, particularly having a length of 5 to 50 amino acid residues, particularly of 10 to 40 amino acid residues and more particularly of 12 to 30 amino acid residues.

In certain embodiments, the peptidic linker comprises amino acid residues selected from Gly, Glu and Ser particularly in an amount of at least about 50%, at least about 60%, at least about 70%, at least about 80%, at least about 90% or 100%. More particularly, the linker comprises a sequence of 1-3 Gly residues interrupted by 1-2 Glu and/or Ser residues. Even more particularly, the linker comprises a sequence of 1-3 Gly residues interrupted by 1 Glu and/or Ser residue.

In certain embodiments, the first VHH antibody competes with a VHH antibody selected from Re5F10, Re6B07, Re7E02 or Re9F06, particularly Re9F06. In certain embodiments, the first VHH antibody is selected from a VHH antibody comprising a VHH sequence as shown in SEQ. ID NO 29 (Re5F10), 248 (Re9F06), 268 (Re22D04), 276 (Re25H10), 260 (Re21D01), 284 (Re26E09) or 288 (Re26E11) or a VHH antibody, which is a variant thereof. In certain embodiments, 1, 2, 3, 4, 5, 6, 7, 8, 9 or 10 amino acids of the VHH reference sequence are replaced by another amino acid.

In certain embodiments, the second VHH antibody competes with a VHH antibody selected from Re5D06, Re6B06, Re6D06, Re6H06, Re9B09, Re9C08, or Re9H01, particularly Re5D06. In certain embodiments, the second VHH antibody comprises a VHH sequence as shown in SEQ. ID NO: 17 (Re5D06) or a variant thereof as described above, e.g. SEQ. ID NO: 232 (Re5D06R28D), SEQ. ID NO: 240 (Re5D06R15_3QE) or SEQ. ID NO: 244 (Re5D06R28_3QE), or in SEQ. ID NO: 49 (Re6B06), SEQ. ID NO: 57 (Re6D06), SEQ. ID NO: 81 (Re6H06), SEQ. ID NO: 129 (Re9B09), SEQ. ID NO: 141 (Re9C08), or SEQ. ID NO: 157 (Re9H01), SEQ. ID NO: 272 (Re22E05) or SEQ. ID NO: 280 (Re26D07) or a VHH antibody, which is a variant thereof. In certain embodiments, 1, 2, 3, 4, 5, 6, 7, 8, 9 or 10 amino acids of the VHH reference sequence are replaced by another amino acid.

In certain embodiments, the second antibody is selected from Re5D06 or a variant thereof, in particular a thermostable or hyperthermostable variant thereof as described above such as ReD05R28.

In certain embodiments, the first VHH antibody is positioned at the N-terminus of the heterodimer and the second VHH antibody is positioned at the C-terminus of the heterodimer. Alternatively, the first VHH antibody is positioned at the C-terminus of the heterodimer and the second VHH antibody is positioned at the N-terminus of the heterodimer.

Particular embodiments of heterodimeric VHH antibodies are shown in SEQ. ID NO: 292-311.

The inventors exploited that VHH antibodies Re9F06 and Re5D06 can simultaneously bind to the same RBD molecule and that a tandem of the two can bind more avidly than either VHH antibody alone. Re5D06 binds to the main epitope (epitope 1). Re9F06 is hyper-thermostable and binds to epitope 2 that is unaffected by those escape mutations. The inventors fused Re9F06 through a structure-optimized linker to the hyper-thermostable VHH antibody Re5D06R28 described above. The resulting tandem indeed bound the South African, Brazilian, and Californian variants with low picomolar affinity hardly detectable dissociation. For a more stringent test, the inventors produced a quadruple (K417T, L452R, E484K, N501Y) RBD mutant that combines the Brazilian and Californian mutations. The Re9F06-Re5D06R28 tandem bound this extremely mutated RBD remarkably well, with only slow dissociation. The analogous Re9F06-Re9B09 tandem showed even less dissociation from the South African and Brazilian RBD mutants and captured the quadruple RBD mutant essentially irreversibly.

Further, fusions of Re9F06 and Re9B09 and Re9F06 and Re5D06R28D showed ≤10 pM binding to the Indian (Delta) RBD mutant.

VHH heterodimers were also found to be capable of neutralizing the Indian (Delta) RBD mutant at concentrations of 0.5 nM or less or even 0.1 nM or less, in particular, fusions of Re9F06 and Re5D06 or variants thereof such as Re5D06R28D, Re5D06R15_3QE or Re5D06R28_3QE.

Monomeric VHH Antibodies with Exceptional Mutation Tolerance

To find yet another solution to the mutation problem, the inventors re-selected the immune libraries (after the initial four rounds of anti-wild type RBD selection) with the above-mentioned quadruple RBD mutant. Sequencing of recovered clones identified a simplified pattern of VHH antibody classes. The Re5F10 VHH antibody class including the VHH antibodies Re5F10, Re21D01, Re26E09 and Re26E11 became very prevalent.

The antibody Re5F10 is hyperthermostable, competes the ACE2-RBD interaction, binds with 30 pM affinity to epitope 2 of either the wildtype or the quadruple mutant RBD. Further, Re6H06 showed 40 pM binding to the Indian (Delta) RBD mutant.

Re6H06 was selected at a lower frequency, but BLI revealed remarkable mutation resistance and 510 pM binding to either the South African or the Brazilian as well as 50 pM binding to the Californian RBD mutant. Further, Re6H06 showed 510 pM binding to the Indian (Delta) RBD mutant.

Finally, the inventors observed a very strong selection for Re9B09 class members with an E115K exchange at CDR3, exemplified by Re9H03. This class targets the main epitope and belongs to the most potent neutralizers. Remarkably, Re9H03 bound essentially irreversibly to the Brazilian, the South African as well as to the quadruple mutant (100-fold better than Re9B09 itself), whereas some dissociation from the Californian single L452R mutant was clearly notable. This indicates that some of the other escape mutations (probably K417N/T) improve VHH antibody binding. As the alpacas were immunized only with the “wild type” RBD, this indicates that their immune system had already generated a diversity of antibodies that “anticipated” even the worst combination of escape mutants.

Homotrimeric VHH Antibodies

Under some circumstance, it might be desirable to engineer VHH antibodies to higher binding strength, e.g. the aforementioned hyper-thermostable VHH antibody Re6B06. The binding strength of an antibody or any other binder is in the first instance ruled by the Kd of the relevant interaction (dissociation constant or the ratio of off and on rates). If the free concentration of the binder equals the Kd, then half of the binding sites are blocked. As a SARS-CoV-2 viral particle contains >100 spike trimers (Bar-On et al., 2020), quantitative neutralization probably only occurs if their majority, i.e. 90% or even 99% are blocked. This requires the binder to be present at concentrations that are 10-fold or a 100-fold higher than the Kd.

A solution to this problem is to fuse the monovalent VHH antibody to the Fc part of an immunoglobulin, e.g. a human IgG and thus make the VHH bivalent. This could impose a stronger binding by avidity effects, but only if the distance of binding sites fits the geometry of the antibody. Apart from this reservation, Fc fusions are associated with additional potential drawbacks: First, production of such Fc fusions should be carried out in human cells to ensure proper folding and a human glycosylation pattern. Second, the Fc fragment is an immune effector domain that might aggravate the problem of inappropriate complement activation and cytokine storms, which are hallmarks of the most severe SARS-CoV-2 pathologies. Third, a dimeric IgG might well dock to two of the spike molecules of a homotrimeric spike, but this would leave the third one in an unbound state.

Thus, the inventors have considered that a homotrimeric ligand would be far more appropriate for blocking a homotrimeric target (FIG. 7). This principle of ‘symmetry-matching’ is a central element of this approach.

Homotrimerization of a VHH antibody requires an appropriate polypeptide fusion partner as trimerization module. According to the present invention, a fusion partner is preferred, comprising at least one of the following features:
(1) it has a length of about 250 amino acids or less, of about 200 amino acids or less, of about 150 amino acids or less or of about 120 amino acids or less, particularly between about 40 and 100 amino acids, e.g. about 90 amino acids;
(2) it is capable of folding properly in commonly used recombinant expression systems (bacteria, yeast, insect and human cells), and assemble into trimers with a very low Kd of e.g. less than 1 nM in the picomolar range—to avoid disassembly at low concentrations; and
(3) it is of mammalian, particularly of human origin to be as non-immunogenic as possible.

Preferred trimerization modules include domains derived from collagen, the most abundant class of proteins of the human body, including the trimerization (NC1) domains of collagen XV (PDB 3N3F, 54 residues) (Wirz et al., 2011), collagen XVIII (PDB 3HSH; 54 residues) (Boudko et al., 2009) and collagen X (PDB 1GR3; 160 amino acids) (Bogin et al., 2002). Another very appropriate homotrimeric fusion partner is the lung surfactant protein D (LSFPD) that recognizes sugars on viral and bacterial pathogens (see e.g. PDB 1B08, 3IKN, 3IKQ, 3IKR). As the SARS-CoV-2 spike protein is heavily glycosylated, a VHH-LSPD fusion could confer a bimodal inhibition of spike function.

The collagen XVIII NC1 (ColXVIII) module has been used for trimerizing VHHs before (Alvarez-Cienfuegos et al., 2016), however, not for the purpose of symmetry-matching and targeting homotrimeric targets. Further, this earlier study used expression in human cells.

In a particular embodiment, the trimerization module comprises an amino acid sequence of SEQ. ID NO: 210, or an amino acid sequence having an identity of at least about 80%, of at least about 90% or of at least about 95% thereto.

The inventors now fused 27 different VHH antibodies through a flexible spacer to this trimerization module and found good expression and solubility in E. coli, and succeeded in just a single purification step to obtain an essentially pure fusion protein, using the technology of cleavable His-tags (Frey and Görlich, 2014; Pleiner et al., 2015).

Amino acid sequences of individual subunits of specific homotrimeric VHH antibodies are shown in SEQ. ID NO: 211 (Re6B06 ColXVIII trimer), SEQ. ID NO: 212 (Re7H02 ColXVIII trimer), SEQ. ID NO: 213 (KG4B11 ColXVIII trimer), SEQ. ID NO: 214 (Re6D06 ColXVIII trimer), SEQ. ID NO: 215 (Re6A11/Re9F06 ColXVIII trimer), and SEQ. ID NO: 216 (Re5A08 ColXVIII trimer).

According to the present invention, it is preferred that the VHH antibody is linked to the trimerization domain via a spacer comprising at least one of the following features:
(1) it is long enough to allow a simultaneous binding of all three VHH copies to the same spike trimer, e.g. having a length of at least about 5 amino acids, particularly about 5 amino acids to about 100 amino acids, and more particularly of about 10 to about 50 amino acid, also depending on the specific target structure;
(2) it is substantially free (i.e. comprising an amount of 10% or less, 5% or less or 2.5% or less) or it is free from amino acid residues selected from Asn, Lys, Arg, Trp, Tyr, Phe, Met, Leu, Ile, Val, His and/or Cys;
(3) it does not contain a strong B-cell epitope;
(4) it is not particularly protease-sensitive, e.g. it does not contain a trypsin or chymotrypsin cleavage site;
(5) it does not contain repetitive sequence portions, e.g. repetitive sequence portions having a length of at least 3 amino acid residues;
(6) it comprises a negative net charge;
(7) it comprises amino acids selected from Gly, Ser, Ala, Thr, Pro, Asp and Glu, (preferably when expressed in a bacterium) or it comprises amino acids selected from Gly, Glu, Ser and Pro (preferably when expressed in a yeast), particularly in an amount of at least about 50%, at least about 60%, at least about 70%, at least about 80%, at least about 90% or 100%; and
(8) it comprises a succession of tripeptides, wherein the first and the second amino acid are selected from Gly, Ser, Ala, Thr, and Pro, particularly Gly, Ser, Ala and Thr, and more particularly Gly, Ser and Ala, and the third amino acid is selected from Asp and Glu; and particularly without direct repetitions of the concatenated tripeptide motifs.

It should be note that different arrangements for the subunits of homotrimeric VHH antibodies are possible. As just described, the trimerization module might be located N-terminally, followed by the spacer and the VHH antibody. Alternatively, the VHH antibody might be located N-terminally, followed by spacer and trimerization module. The best arrangement will depend on the geometry of the target to be neutralized.

In particular embodiments, the present invention relates to a neutralizing homotrimeric VHH antibody, e.g. a VHH antibody which has a lowest SARS-CoV-2 neutralizing concentration of about 100 pM or less, of about 25 pM or less, of about 17 pM or less, of about 10 pM or less, of about 5 pM or less or of about 1.7 pM or less.

The inventors tested the principle of avidity enhancement for the rather weakly binding Re6A11 antibody (labelled with two Alexa488 fluorescence groups per VHH module), whose monomeric version is readily washed off its targets and hence showed only very weak staining of Spike molecules in transfected HeLa cells (FIG. 8). The trimerized version, however, showed a strong signal even when used at lower concentrations and with just one Alexa488 per VHH module. This strong staining persisted also during very excessive washing steps and when the VHH trimer was used at a very low concentration (1 nM).

As second proof of principle for the avidity enhancement, the inventors used the virus neutralization assay as described above. Table 4 shows the minimum SARS-CoV-2-neutralizing concentrations of selected nanobody monomers and trimers. Virus neutralization experiments were performed as in FIG. 4, with VHH concentrations being titrated down in factor \sqrt{10} steps. Lowest SARS-CoV-2-neutralizing concentrations of selected VHH monomers and trimers are listed that fully suppressed infection over a period of three days. Concentrations refer to the respective VHH units. The trimers were tandem fusions of VHH, a 39 residues spacer followed by the collagen XVIII NC1 domain.

The monomeric version of the VHH antibody Re5A08 blocked infection down to a concentration of 5 nM. In trimerized form, however, the block was still tight at the lowest tested concentration of 17 pM (calculated for the VHH concentration). As lower concentrations have not yet been tested, the enhancement by trimerization is most probably still underestimated. In fact, for the given geometry and length of spacers (39 residues in the present example), an increase in binding strength by a factor of 10² or even more, e.g. up to 10⁶-10⁹ (dependent on the specific assumptions) is possible. In such regime and for any reasonably strong binder, the binding constant will no longer be limiting, but only the on-rate for the binding reaction.

In the case of SARS-CoV-2 neutralization using either VHH Re6D06, Re9B09, Re9H01, or Re7H02, the inventors found that VHH-spacer-collagen XVIII NC1 fusions are particularly efficient. For these fusions, the inventors found an extreme (30 000-fold) gain in potency of VHH antibody Re6B06. This VHH neutralizes as a monomer down to concentrations of 50 nM. As a trimer, however, 1.7 pM were sufficient for complete (IC99+) neutralization (concentrations calculated for the VHH moiety). This corresponds to 40 ng VHH fusion per liter. The Re6B06-spacer-ColXVIII fusion thus outperformed (along with the class-related and equally effective Re7H02 and KGB11 trimers) all other tested variants (Table 4).

Even, VHH antibodies, which neutralize as monomers already very potently, benefit from the trimerization. The trimer of Re6D06, for example, neutralized 100-fold better (at 17 pM) than the corresponding monomer (1.7 nM). The super-neutralizing VHH Re5D06 (or other members of the same class) showed an at least 3-fold improvement, they fully suppressed infection at a concentration of 50 pM. This corresponds to ˜0.7 μg VHH antibody per liter.

When injected into a patient, and generously assuming that it gets diluted into an accessible volume of 50 l, then 100 μg should already be sufficient to reach a therapeutically effective concentration in the extracellular space. As also Re5D06 is being combined with an avidity-enhancing trimerization module, there is a comfortable safety margin in this estimate. Following optimization, we usually obtain one gram of nanobody from 100 g E. coli biomass. Thus, an industry-standard 1000 l fermenter might yield >1 million therapeutic doses within a single run.

Further, the inventors tested how the trimerization according to the present invention compares to an Fc-mediated VHH dimerization, using a recently published VHH antibody VHH-72 (SEQ. ID NO: 217) as an example (Wrapp et al., 2020). VHH-72 was originally raised and selected against SARS-CoV-1, but it cross-reacts with SARS-CoV-2 although with a 30-fold lower affinity. 20 mg/liter VHH-72 monomer were shown by (Wrapp et al., 2020) to be required for a complete neutralization of SARS-CoV-2. The Fc dimer has been reported to neutralize down to 2 mg/liter (complete neutralization)—a 10-fold improvement (Wrapp et al., 2020).

In the test system used herein, 500 nM (7 mg/liter) of the VHH-72 monomer were required for a complete neutralization (Table 4); a partial neutralization was seen down to 170 nM (FIG. 7C). The VHH-72 collagen XVIII NC1 trimer (SEQ. ID NO: 218), however, showed complete neutralization down to 1.2 μg/liter (50 pM). This trimer is thus 6 000 to 17 000-fold more potent than the monomer and 1 700-fold more potent than the Fc dimer. In other words, the trimer outperforms the traditional Fc fusion by a large margin. The experiment is also a proof of concept for the symmetry-matching strategy for a VHH that originally targeted a different molecule than the SARS-CoV-2 spike.

Thus, in a particular embodiment, the present invention relates to a homotrimeric VHH antibody selected from:
(a) the VHH antibody VHH-72 comprising an VHH amino acid sequence as shown in SEQ. ID NO: 217, or
(b) a sequence which has an identity of at least 70%, at least 80%, at least 90%, at least 95% or at least 99% to a VHH sequence of (a).

In a further particular embodiment, the present invention relates to a homotrimeric VHH antibody comprising a subunit as shown in SEQ. ID NO: 218.

TABLE 4

| | | Lowest neutralizing | | |
|---|---|---|---|---|
| | Seq | VHH concentration | | kDa/ |
| VHH | ID | pM | μg/liter | chain |
| Re6B06 | 49 | 50 000.0 | 750.00 | 12 |
| Re6B06 trimer | 211 | 1.7 | 0.04 | 22 |
| Re7H02 | 105 | 17 000.0 | 250.00 | 12 |
| Re7H02 trimer | 212 | 1.7 | 0.04 | 22 |
| Re6D06 | 57 | 1 700.0 | 25.00 | 13 |
| Re6D06 trimer | 214 | 17.0 | 0.40 | 23 |
| Re6A11 | 41 | 50 000.0 | 750.00 | 13 |
| Re6A11/Re9F06 trimer | 215 | 170.0 | 4.00 | 23 |
| Re5A08 | 1 | 5 000.0 | 75.00 | 13 |
| Re5A08 trimer | 216 | 17.0 | 0.40 | 23 |
| VHH-72* | | 1 400 000.0 | 20 000.00 | 14 |
| VHH-72 Fc dimer* | | 150 000.0 | 2 000.00 | 50 |
| VHH-72** | 217 | 500 000.0 | 7 000.00 | 14 |
| VHH-72 trimer** | 218 | 50.0 | 1.20 | 23 |

Indicated VHH versions were tested and scored (IC99+) in the SARS-COV-2 neutralization assay as described above. Listed trimers are all VHH-spacer-Collagen XVIII NC1 fusions with an identical design. Concentrations refer to the VHHs moieties.
*Sequences and neutralization data for the VHH72 monomer and the VHH-72 Fc fusion as reported by Wrapp et al., 2020.
**VHH-72 neutralization from FIG. 7C.

Further, the inventors found that the trimerization module of collagen XVIII NC1 provides more potent SARS-CoV-2 neutralization than the trimerization module of collagen XV NC1. Thus, in certain embodiments, the present invention refers to a homotrimeric VHH antibody comprising a collagen XVIII NC1 trimerization module. The collagen XVIII NC1 trimerization module may be located at N-terminal or C-terminal to the VHH antibody, particularly C-terminal to the VHH antibody.

Indeed, the concept of providing homomultimeric, particularly homotrimeric VHH antibodies, is not limited to SARS-CoV-2. It is quite remarkable that many viruses rely on homotrimeric entry receptors and/or fusion proteins with 03 rotational symmetry. The symmetry-mismatch to bivalent antibodies is perhaps of advantage for those viruses when it comes to delaying a neutralizing immune response. Thus, our symmetry-matching approach to homotrimeric neutralizing VHH antibodies is also suitable against any target structure having a threefold-rotationally symmetric subunit structure. Such a target structure is present on other viruses, such as other members of the Coronaviridae, Orthomyxoviridae (with their trimeric HA-proteins), many Paramyxoviridae (with their trimeric fusion proteins), Dengue fever- and Zika-viruses (3-fold or higher order protein assemblies on their surface), as well as Herpesviridae (trimeric gB fusion protein) or Human Immunodeficiency Virus (with its trimeric gp120 protein).

Moreover, the approach can even be extended to efficiently inhibit non-viral targets that also show a threefold rotational symmetry, such as the members of the TNF Ligand and TNF Receptor superfamilies, e.g. the death ligands and receptors that trigger the extrinsic pathway of apoptosis (e.g. CD95, TNFα, or Trail). In addition, here, a stable and non-immunogenic trimerization module as the ones derived from human collagens X, XV, or XVIII may be used.

Thus, a further embodiment of the present invention is a homotrimeric VHH antibody direct to a target structure, which is different from spike protein S1 domain, particularly from the receptor-binding domain (RBD) of the SARS-CoV-2 S1 domain.

The lengths of spacers between VHHs and trimerization unit may be dependent from the specific target structure and thus subject to optimization: If a spacer is too short, then it will not allow all three binding sites to be used at the same time or cause tension in the bound state, while a spacer that is too long might be entropically suboptimal in terms of increasing the local VHH concentration. Based on the above teaching, an optimal spacer may be developed, which will not only provide the maximum enhancement in binding strength but also a gain specificity as multivalent binding is most favored to objects of a complementary geometry.

Production of Monomeric, Heterodimeric and Homomultimeric, Particularly Homotrimeric VHH Antibodies

A further aspect of the present invention relates to a method of producing VHH antibodies including monomeric, heterodimeric and homomultimeric, particularly homotrimeric VHH antibodies. This method relates to the production of VHH antibodies directed against any target structure including, but not limited to the monomeric, heterodimeric or homotrimeric VHH antibodies as described herein above. In particular, this method also includes the production of homodimeric VHH antibodies, wherein the VHH antibody sequence is fused to a dimerization module, e.g. an Fc immunoglobulin domain.

A VHH antibody may be recombinantly produced in a suitable host cell, e.g. in a prokaryotic or eukaryotic host cell or host organism. For this purpose, a nucleic acid molecule encoding the VHH antibody is introduced into the host cell or host organism, and expressed in the host cell or host organism. The nucleic acid molecule may encode a monomeric VHH antibody or a subunit of a homomultimeric VHH antibody, particularly of a homotrimeric VHH antibody.

Any therapeutic application of neutralizing anti-SARS-CoV-2 VHH antibodies requires their production at a large scale. In this aspect, production in bacteria or yeast, e.g., Pichia pastoris, Saccharomyces cerevisiae, or Hansenula polymorpha, may be preferred. Production in yeast, e.g. Pichia pastoris, Saccharomyces cerevisiae, or Hansenula polymorpha, is particularly preferred because it ensures a quantitative formation of disulfide bonds, allows for excellent yields (in the range of grams per liter culture), while the secretion into the medium allows the subsequent purification to begin with only few contaminating proteins.

In a particular embodiment, the VHH antibody is recombinantly produced in a bacterium, e.g. E. coli or Bacillus. For example, expression in a bacterium may involve cytoplasmic and/or periplasmic expression and purification of the VHH antibody from the host cell, or secretory expression and purification of the VHH antibody from the culture medium. In certain embodiments, the nucleic acid sequence encoding the VHH antibody is fused to at least one sequence directing the expression to the periplasm and/or into the culture medium.

In a further particular embodiment, the VHH antibody is recombinantly produced in a eukaryotic host cell or host organism, preferably in yeast, e.g., in Pichia pastoris, Saccharomyces cerevisiae, or Hansenula polymorpha. For example, expression in a eukaryotic host cell or host organism, e.g., yeast may involve cytoplasmic and/or periplasmic expression and purification of the VHH antibody from the host cell, or preferably secretion from the host cell and purification of the VHH antibody from the culture medium. In certain embodiments, the nucleic acid sequence encoding the VHH antibody is fused to at least one sequence directing the expression into the culture medium.

The VHH antibody encoding nucleic acid molecule may comprise a sequence encoding a monomeric VHH antibody, a sequence encoding a heterodimeric VHH antibody or a sequence encoding a subunit of a multimeric VHH antibody, particularly of a trimeric VHH antibody. The sequence encoding a heterodimeric VHH antibody may comprise two sequences each encoding a monomeric VHH antibody arranged in tandem fused to each other optionally through a linker. The subunit sequence may comprise (from N-terminus to C-terminus) the VHH antibody sequence, optionally a spacer, and a multimerization domain, particularly a trimerization domain, e.g. the Collagen XVIII NC1 domain. Alternatively, the subunit sequence may comprise (from N-terminus to C-terminus) a multimerization domain, particularly a trimerization domain, optionally a spacer and the VHH antibody sequence.

Thus, a further aspect of the present invention relates to a method for the recombinant production of a monomeric VHH antibody, a heterodimeric VHH antibody or a homomultimeric, e.g., a homotrimeric VHH antibody, particularly a homotrimeric VHH antibody as described herein, comprising cultivating a cell or an organism, particularly a yeast such as Pichia pastoris and obtaining the VHH antibody from the cell or organism or from the medium, particularly from the medium. The cell or organism comprises a nucleic acid molecule encoding the monomeric VHH antibody, the heterodimeric VHH antibody or a subunit of the homomultimeric, particularly homotrimeric VHH antibody in operative linkage with a suitable expression control sequence. The nucleic acid molecule may be present in extrachromosomal form or integrated into the genome of the host cell or organism.

In particular embodiments, the VHH antibody sequence or the multimer, e.g. trimer subunit sequence is fused to an N-terminal, cleavable sequence, which confers secretion in the respective host cell or organism.

For bacteria, the sequence, which confers secretion, may comprise a cleavable signal peptide sequence. Examples include the signal peptides of E. coli DsbA, DsbC, DsbG or Pectobacterium carotovorum PelB.

For eukaryotes, e.g. yeast, the sequence, which confers secretion, may comprise a signal peptide sequence (pre-sequence) and optionally a propeptide sequence (pro-sequence). In certain embodiments, the secreted VHH or VHH trimer is synthesized in a yeast with an N-terminal signal peptide sequence followed by a propeptide sequence.

In certain embodiments, the signal peptide sequence (pre-sequence) directs the protein into the lumen of the endoplasmic reticulum (ER) and is cleaved during membrane-passage. The propeptide sequence serves as a forward secretion signal that enhances secretion from the cell into the culture medium, for example, it may favor packaging into COPII vesicles, export from the ER, and transit through the secretory pathway. The propeptide sequence may be cleaved from the mature protein, e.g. by Kex2 in the late Golgi. A suitable example is the pre-propeptide of S. cerevisiae alpha-factor, which comprises a post-translational signal peptide (the pre-sequence) as well as a pro-peptide (Oka et al., 1999). The inventors observed that this fusion strategy, using the (posttranslational) alpha factor signal peptide, allowed for good production of the VHH antibodies Re5D06 or Re9B09, and also of a trimeric Re5D06-spacer-Collagen XVIII NC1 fusion construct. However, no secretion was detectable for other tested VHH-trimers, including trimers of the Re6B06 class of VHH antibodies.

In further embodiments, a co-translational signal peptide sequence is used, which initiates transport already on nascent chain-ribosome complexes. The Ost1 signal peptide sequence belongs to this category and confers transport into the ER in an SRP/Sec65-dependent but Sec62- and Sec63-independent manner (Willer et al., 2008; Fitzgerald and Glick, 2014). The inventors hence constructed Pichia strains carrying expression cassettes for fusions of the Ost1 signal peptide, the alpha factor propeptide, a VHH antibody, a long spacer as described above, and the collagen XVIII NC1 trimerization domain. With such a design, all tested trimeric VHH fusions were efficiently secreted into the medium.

Thus, a particular embodiment of the invention relates to a method for the recombinant production of a monomeric VHH antibody, a heterodimeric VHH antibody or a homomultimeric, homotrimeric VHH antibody, particularly a homotrimeric VHH antibody as described herein, comprising cultivating a yeast such as Pichia pastoris, Saccharomyces cerevisiae, or Hansenula polymorpha, and obtaining the monomeric, heterodimeric or homomultimeric, e.g. homotrimeric VHH antibody from the medium, wherein the yeast comprises a nucleic acid molecule encoding (from N-terminus to C-terminus) (i) a cleavable co-translational signal sequence (pre-sequence), i.e. a signal sequence, which initiates transport on nascent chain-ribosome complexes, (ii) optionally a cleavable pro-sequence, which enhances secretion, e.g. an ER export signal or forward secretion signal sequence, and (iii) the VHH antibody sequence, e.g., the monomeric VHH antibody, the heterodimeric VHH antibody or a subunit of the homomultimeric, e.g. homotrimeric VHH antibody, particularly a fusion comprising the VHH antibody sequence, optionally a spacer, and a trimerization domain, e.g. the Collagen XVIII NC1 domain. Preferably, the VHH antibody sequence is located N-terminal from the multimerization domain, e.g., trimerization domain. In certain embodiments, the multimerization domain, e.g. trimerization domain may be located N-terminal from the VHH antibody sequence.

In particular embodiments, the N-terminal signal peptide sequence (i) initiates co-translational (i.e. SRP/Sec65-dependent but Sec62- and Sec63-independent) transport into the ER. Examples of such sequences include the Ost1 signal sequence of Saccharomyces cerevisiae (SEQ ID. NO: 219) or an Ost1 signal sequence from a related yeast species, e.g. from Pichia pastoris (SEQ ID. NO: 220), Schizosaccharomyces pombe (SEQ. ID. NO: 221), or Candida albicans (SEQ ID. NO: 222) or a variant of those, e.g. having an amino acid identity of at least 80%, of at least 90% or of at least 95% thereto.

In particular embodiments, the pro-sequence (ii) provides a forward secretion signal. Examples of such sequences include the propeptide of S. cerevisiae alpha-factor (SEQ ID. NO: 223) or a variant thereof, having an amino acid identity of at least 80%, of at least 90% or of at least 95%, or another Kex2-cleavable pro-peptide that promotes packaging into COPII vesicles and thus export from the ER.

Further, the invention relates to a nucleic acid molecule encoding a polypeptide comprising (from N-terminus to C-terminus) (i) a cleavable co-translational signal sequence (pre-sequence), i.e. a signal sequence, which initiates transport on nascent chain-ribosome complexes, (ii) optionally a cleavable pro-sequence, which enhances secretion, e.g., an ER export signal or forward secretion signal sequence, and (iii) a VHH antibody sequence, e.g., a monomeric VHH antibody, a heterodimeric VHH antibody or a subunit of a homomultimeric, e.g., homotrimeric VHH antibody.

Furthermore, the invention relates to a polypeptide encoded by the nucleic acid molecule as described above, particularly a polypeptide comprising (from N-terminus to C-terminus) (i) a cleavable co-translational signal sequence (pre-sequence), i.e. a signal sequence, which initiates transport on nascent chain-ribosome complexes, (ii) optionally a cleavable pro-sequence, which enhances secretion, e.g. an ER export signal or forward secretion signal sequence, and (iii) the VHH antibody sequence, e.g., a monomeric VHH antibody, a heterodimeric VHH antibody or a subunit of a homomultimeric, e.g., homotrimeric VHH antibody.

Therapeutic Applications

Still a further aspect of the present invention is the use of an active agent as described above selected from a monomeric VHH antibody, a heterodimeric VHH antibody, a set of at least 2 different VHH antibodies, or a multimeric, particularly trimeric VHH antibody in medicine, particularly for therapeutic and/or in vitro or vivo diagnostic use. In certain embodiments, the monomeric VHH antibody, the heterodimeric VHH antibody, the set of VHH antibodies or the multimeric, particularly trimeric VHH antibody is used in human medicine.

In particular embodiments, a therapeutic VHH antibody is selected from (i) a VHH antibody comprising the CDR3 sequence of VHH antibody Re5D06 or a related sequence, particularly the CDR3 sequence of a thermostable or hyperthermostable variant thereof, e.g. the VHH antibody Re5D06R11, Re5D06R13, Re5D06R15, Re5D06R28, Re5D06R28D, Re5D06R15_3QE or Re5D06R28D_3Q (ii) a VHH antibody comprising the CDR1, CDR2 and CDR3 sequences of VHH antibody Re5D06 or related sequences, particularly the CDR1, CDR2 or CDR3 sequence of a thermostable or hyperthermostable variant thereof, e.g. the VHH antibody Re5D06R11, Re5D06R13, Re5D06R15, Re5D06R28, Re5D06R28D, Re5D06R15_3QE or Re5D06R28D_3Q (iii) a VHH antibody comprising the VHH sequence of VHH antibody Re5D06 or a related sequence, particularly the VHH sequence of a thermostable or hyperthermostable variant thereof, e.g. the VHH antibody Re5D06R11, Re5D06R13, Re5D06R15, Re5D06R28, Re5D06R28D, Re5D06R15_3QE or Re5D06R28D_3Q or (iv) a VHH antibody competing with VHH antibody Re5D06. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of the hyperthermostable VHH antibody Re6H06 or a related sequence, (ii) a VHH antibody comprising the CDR1, CDR2 and CDR3 sequences of the hyperthermostable VHH antibody Re6H06 or related sequences, (iii) a VHH antibody comprising the VHH sequence of the hyperthermostable VHH antibody Re6H06 or a related sequence, or (iv) a VHH antibody competing with VHH antibody Re6H06. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of the thermostable VHH antibody Re9B09 or a related sequence, (ii) a VHH antibody comprising the CDR1, CDR2 and CDR3 sequences of the thermostable VHH antibody Re9B09 or related sequences, (iii) a VHH antibody comprising the VHH sequence of the thermostable VHH antibody Re9B09 or a related sequence, or (iv) a VHH antibody competing with VHH antibody Re9B09. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of VHH antibody Re9H01 or a related sequence, (ii) a VHH antibody comprising the CDR1, CDR2 and CDR3 sequences of VHH antibody Re9H01 or related sequences, (iii) a VHH antibody comprising the VHH sequence of VHH antibody Re5D06 or a related sequence, or (iv) a VHH antibody competing with VHH antibody Re9H01. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of VHH antibody Re6B06 or a related sequence, particularly the CDR3 sequence of a hyperthermostable variant thereof, e.g. the VHH antibody KGB4B11 (ii) a VHH antibody comprising the CDR1, CDR2 and CDR3 sequences of VHH antibody Re6H06 or related sequences, particularly the CDR1, CDR2 and CDR3 sequences of a hyperthermostable variant thereof, e.g. the VHH antibody KGB4B11 (iii) a VHH antibody comprising the VHH sequence of VHH antibody Re6H06 or a related sequence, particularly the VHH sequence of a hyperthermostable variant thereof, e.g. the VHH antibody KGB4B11 or (iv) a VHH antibody competing with VHH antibody Re6H06 and/or KGB4B11. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of VHH antibody Re7E02 or a related sequence, (ii) a VHH antibody comprising the CDR1, CDR2 and CDR3 sequences of VHH antibody Re7E02 or related sequences, (iii) a VHH antibody comprising the VHH sequence of VHH antibody Re7E02 or a related sequence, or (iv) a VHH antibody competing with VHH antibody Re7E02. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of an VHH antibody of the Re9B09 class, particularly Re9H03 and even more particularly Re22E05 or a related sequence, (ii) a VHH antibody comprising the CDR1, CDR2 and CDR3 sequences of VHH antibody Re9H03 or Re22E05 or related sequences, (iii) a VHH antibody comprising the VHH sequence of VHH antibody Re9H03 or Re22E05 or a related sequence, or (iv) a VHH antibody competing with VHH antibody Re9H03 or Re22E05. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of an VHH antibody of the Re6H06 class, particularly ReH06 or Re26D07 or a related sequence, (ii) a VHH antibody comprising the CDR1, CDR2 and CDR3 sequences of VHH antibody ReH06 or Re26D07 or related sequences, (iii) a VHH antibody comprising the VHH sequence of VHH antibody ReH06 or Re26D07 or a related sequence, or (iv) a VHH antibody competing with VHH antibody ReH06 or Re26D07. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of VHH antibody Re21 D01 or a related sequence, (ii) a VHH antibody comprising the CDR1, CDR2 and CDR3 sequences of VHH antibody Re21 D01 or related sequences, (iii) a VHH antibody comprising the VHH sequence of VHH antibody Re21 D01 or a related sequence, or (iv) a VHH antibody competing with VHH antibody Re21D01. In certain embodiments, 1, 2, 3, 4, 5, 6, 7, 8, 9 or 10 amino acids of the VHH sequence of the reference antibody are replaced by another amino acid.

In particular embodiments, a therapeutic VHH antibody is selected from (i) a VHH antibody comprising the CDR3 sequence of VHH antibody Re21H01 or a related sequence, (ii) a VHH antibody comprising the CDR1, CDR2 and CDR3 sequences of VHH antibody Re21H01 or related sequences, (iii) a VHH antibody comprising the VHH sequence of VHH antibody Re21H01 or a related sequence, or (iv) a VHH antibody competing with VHH antibody Re21H01. In certain embodiments, 1, 2, 3, 4, 5, 6, 7, 8, 9 or 10 amino acids of the VHH sequence of the reference antibody are replaced by another amino acid.

In particular embodiments, a therapeutic heterodimeric VHH antibody comprises a first VHH antibody selected from (i) a VHH antibody comprising the CDR3 sequence of VHH antibody Re25H10 or a related sequence, (ii) a VHH antibody comprising the CDR1, CDR2 and CDR3 sequences of VHH antibody Re25H10 or related sequences, (iii) a VHH antibody comprising the VHH sequence of VHH antibody Re25H10 or a related sequence, or (iv) a VHH antibody competing with VHH antibody Re25H10. In certain embodiments, 1, 2, 3, 4, 5, 6, 7, 8, 9 or 10 amino acids of the VHH sequence of the reference antibody are replaced by another amino acid.

In particular embodiments, a therapeutic heterodimeric VHH antibody comprises a first VHH antibody selected from (i) a VHH antibody comprising the CDR3 sequence of VHH antibody Re9F06 or a related sequence, (ii) a VHH antibody comprising the CDR1, CDR2 and CDR3 sequences of VHH antibody Re9F06 or related sequences, (iii) a VHH antibody comprising the VHH sequence of VHH antibody Re21H04 or a related sequence, or (iv) a VHH antibody competing with VHH antibody Re9F06. In certain embodiments, 1, 2, 3, 4, 5, 6, 7, 8, 9 or 10 amino acids of the VHH sequence of the reference antibody are replaced by another amino acid.

In particular embodiments, a therapeutic heterodimeric VHH antibody comprises a first VHH antibody selected from (i) a VHH antibody comprising the CDR3 sequence of VHH antibody Re5F10 or a related sequence, (ii) a VHH antibody comprising the CDR1, CDR2 and CDR3 sequences of VHH antibody Re5F10 or related sequences, (iii) a VHH antibody comprising the VHH sequence of VHH antibody Re5F10 or a related sequence, or (iv) a VHH antibody competing with VHH antibody Re5F10. In certain embodiments, 1, 2, 3, 4, 5, 6, 7, 8, 9 or 10 amino acids of the VHH sequence of the reference antibody are replaced by another amino acid.

In particular embodiments, a therapeutic heterodimeric VHH antibody comprises a first VHH antibody selected from (i) a VHH antibody comprising the CDR3 sequence of VHH antibody Re5F10, Re22D04, Re25H10, Re21D01, Re26E09 or Re26E11 or a related sequence, (ii) a VHH antibody comprising the CDR1, CDR2 and CDR3 sequences of VHH antibody Re5F10, Re22D04, Re25H10, Re21D01, Re26E09 or Re26E11 or related sequences, (iii) a VHH antibody comprising the VHH sequence of VHH antibody Re21H04 or a related sequence, or (iv) a VHH antibody competing with VHH antibody Re5F10, Re22D04, Re25H10, Re21D01, Re26E09 or Re26E11. In certain embodiments, 1, 2, 3, 4, 5, 6, 7, 8, 9 or 10 amino acids of the VHH sequence of the reference antibody are replaced by another amino acid.

In particular embodiments, a therapeutic heterodimeric VHH antibody comprises a second VHH antibody selected from (i) a VHH antibody comprising the CDR3 sequence of VHH antibody Re5D06 or a related sequence, e.g. the CDR3 sequence of Re5D06R15, Re5D06R28, Re5D06R28D, Re5D06R15_3QE or Re5D06R28_3QE (ii) a heterodimeric VHH antibody comprising the CDR1, CDR2 and CDR3 sequences of VHH antibody Re5D06 or related sequences, (iii) a heterodimeric VHH antibody comprising the VHH sequence of VHH antibody Re5D06 or a related sequence, or (iv) a heterodimeric VHH antibody competing with VHH antibody Re5D06. In certain embodiments, 1, 2, 3, 4, 5, 6, 7, 8, 9 or 10 amino acids of the VHH sequence of the reference antibody are replaced by another amino acid.

In particular embodiments, a therapeutic heterodimeric VHH antibody comprises first VHH antibody and a second VHH antibody as described above.

Therapeutic applications of an active agent as described above, e.g. a monomeric VHH antibody, a heterodimeric VHH antibody, a set of at least 2 different VHH antibodies, or a multimeric, particularly trimeric VHH antibody include methods for preventing or treating a disorder caused by and/or associated with a Coronavirus infection, particularly a disorder caused by and/or associated with a SARS-CoV-2 infection and more particularly of preventing or treating COVID-19. The therapeutic applications also include methods for preventing or treating a disorder caused by and/or associated with an infection by a SARS-CoV-2 mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.

As outlined, above, however, the trimeric VHH antibody may be directed to other target structures, and thus, is useful in methods for preventing or treating a disorder caused by and/or associated with a virus infection, particularly a disorder caused by and/or associated with a Coronavirus infection, an Orthomyxovirus, e.g. an Influenza A, B, or C virus, a Paramyxovirus, e.g. a Parainfluenza virus, Respiratory Syncytial virus, Measles virus, or Mumps virus, Dengue fever virus, Zikavirus, a Herpesvirus or HIV. Further, the trimeric VHH antibody is useful in methods for preventing or treating a non-viral disorder, particularly a disorder caused by and/or associated with a member of the TNF ligand superfamily and/or a member of the TNF Receptor superfamily.

In therapeutic applications, the active agent is administered in an effective amount to a subject in need thereof, particularly to a human subject. The dose will depend from the specific type of agent, e.g. monovalent VHH antibody or multimeric, particularly trimeric VHH antibody, and the type of disease.

In the prevention or treatment of COVID-19, a daily dose of a monomeric VHH antibody for a human subject is any suitable dose based on the severity of the disease, the specific type of agent, and the mode of administration, e.g. locally or systemically, particularly from about 5 μg to about 5000 mg per day.

In the prevention or treatment of COVID-19, a daily dose of a heterodimeric VHH antibody for a human subject is any suitable dose based on the severity of the disease, the specific type of agent, and the mode of administration, e.g. locally or systemically, particularly from about 5 μg to about 5000 mg per day.

In the prevention or the treatment of COVID-19, a daily dose of a trimeric VHH antibody for a human subject is any suitable dose based on the severity of the disease, the specific type of agent, and the mode of administration, e.g. locally or systemically, particularly from about 1 μg to about 2500 mg per day.

In the prevention or treatment of COVID-19, a unit dose of a monomeric VHH antibody for a human subject is any suitable dose based on the severity of the disease, the specific type of agent, and the mode of administration, e.g. locally or systemically, particularly from about 5 μg to about 5000 mg per unit.

In the prevention or treatment of COVID-19, a unit dose of a heterodimeric VHH antibody for a human subject is any suitable dose based on the severity of the disease, the specific type of agent, and the mode of administration, e.g. locally or systemically, particularly from about 5 μg to about 5000 mg per unit.

In the prevention or the treatment of COVID-19, a unit dose of a trimeric VHH antibody for a human subject is any suitable dose based on the severity of the disease, the specific type of agent, and the mode of administration, e.g. locally or systemically, particularly from about 1 μg to about 2500 mg per unit.

Typically, the VHH antibody is administered as a pharmaceutical composition comprising the active agent and a pharmaceutically acceptable carrier or excipient. Examples of suitable carriers and excipients for formulating antibodies or antibody fragments are well known in the art.

Depending on the stage and the severity of the disorder, the pharmaceutical composition may be administered once or several times during the course of the disorder. For example, it may be administered once or several times daily, each second day, two times weekly or weekly for a suitable period of time.

In certain embodiments, the pharmaceutical composition is administered parenterally, e.g. by subcutaneous, intramuscular or intravenous injection or by infusion. In further embodiments, the pharmaceutical composition may be administered locally, e.g. orally, nasally or intrapulmonary, for example by inhalation as an aerosol.

In certain embodiments, the pharmaceutical composition is administered in an early infection stage, e.g. in an infection stage where the upper airways such as the oral cavity, the nasal cavity, the paranasal sinus, the pharynx and/or the throat are infected, but the lower airways such as the bronchi and/or the lung are not infected. In further embodiments, the pharmaceutical composition is administered in a late infection stage wherein the lower airways such as the bronchi and/or the lung are infected. Further, the pharmaceutical composition may be administered in an infection stage where the subject suffers from a respiratory dysfunction and optionally is ventilated. In still further embodiments, the pharmaceutical composition may be administered prophylactically to a subject being at risk of a Coronavirus infection, e.g. a subject, which has previously, for example, within 1, 2, 3, 4, or 5 days or even longer, been in close contact with an infected subject.

The VHH antibody may be administered alone or together with a further active agent, which may be selected from anti-viral agents such as remdesivir or methotrexate.

A further aspect that needs to be considered is the plasma half-life of a VHH antibody. Proteins smaller than 40 kDa are subject to considerable renal clearance, and an isolated VHH antibody (˜15 kDa) shows, therefore, a plasma half-life of only 4 hours (Hoefman et al., 2015). Thus, the administration might comprise repeated daily injections or continuous perfusion during anti-viral therapy that might last for days to weeks. One solution to this problem is to increase the size of the protein. Fusion to spacer and trimerization module increases the size to 701 kDa, and such fusion should then have a far longer plasma half-life that is no longer limited by renal clearance. One can, however, also imagine alternative application routes. In fact, inhalation of an anti-SARS-CoV-2 VHH antibody formulation would bring the therapeutic agent directly to the site of the highest and most problematic virus load, namely the airway epithelia.

The here outlined antiviral clinical strategy will become more robust if not just one but an entire set of several VHH antibodies is deployed. First, one should expect synergistic effects for neutralizing antibodies that bind to non-overlapping sites. Second, patients might develop anti-idiotypic antibodies against a given therapeutic VHH antibody and render it ineffective in treatment. A switch to another VHH would solve the problem. Third, and perhaps most importantly there is the clear risk that SARS-CoV2 evolves further, that escape mutations in its RBD might destroy the epitope of a given VHH antibody and render such VHH inefficient in therapy. This risk can be minimized by utilizing a panel of neutralizing antibodies that recognize non-identical and possibly even non-overlapping epitopes, or by at least switching to another VHH that still neutralizes a given escape mutant.

It will be a critical clinical decision when, during an anti-viral therapy, the treatment regime needs to be adjusted and switched from one VHH antibody to another. It is therefore important to reiterate that the here described therapeutic VHH antibodies are also suitable for diagnostic purposes and can thus guide such decision. When, for example, a mutation in the RBD abolishes the binding and thus impairs the neutralizing power of a given VHH antibody, this can be reported through a diagnostic test using the same VHH. Conversely, if a mutant virus isolate can still be stained with a fluorescent VHH antibody that neutralizes the prototypic SARS-CoV2, then this VHH antibody will also be likely to neutralize the mutant one. The dual therapeutic and diagnostic use of anti-SARS-CoV-2 VHHs is, therefore, central to this invention.

Diagnostic Applications

Diagnostic applications include in vitro methods wherein a VHH antibody is used for detecting Coronavirus spike protein in a sample, e.g. in a body fluid such as saliva, blood, serum or plasma, stool samples or in a tissue or biopsy sample. Diagnostic applications further include in vivo methods wherein a VHH antibody is used for detecting Coronavirus spike protein in a subject, particularly in a human patient. For diagnostic applications the VHH antibody carry a label for direct detection or used in combination with secondary detection reagents, e.g. antibodies, including conventional antibodies or VHH antibodies, for indirect detection according to established techniques in the art.

In particular embodiments, a diagnostic VHH antibody is selected from (i) a VHH antibody comprising the CDR3 sequence of VHH antibody Re6D06 or a related sequence, (ii) a VHH antibody comprising the CDR1, CDR2 and CDR3 sequences of VHH antibody Re6D06 or related sequences, (iii) a VHH antibody comprising the VHH sequence of VHH antibody Re6D06 or a related sequence, or (iv) a VHH antibody competing with VHH antibody Re6D06.

In particular embodiments, a diagnostic VHH antibody is selected from (i) a VHH antibody comprising the CDR3 sequence of VHH antibody Re8E12 or a related sequence, (ii) a VHH antibody comprising the CDR1, CDR2 and CDR3 sequences of VHH antibody Re8E12 or related sequences, (iii) a VHH antibody comprising the VHH sequence of VHH antibody Re8E12 or a related sequence, or (iv) a VHH antibody competing with VHH antibody Re8E12.

Further, the present invention is explained in more detail by the following Figures and Examples.

### FIGURES

FIG. 1: Sequence alignment and highlighting of variable regions

FIG. 1 shows an alignment of VHH sequences from Table 1. Residues that deviate from the consensus are shown in colour. The three variable CDR regions are indicated.

FIG. 2: Staining of transfected cells transiently expressing the SARS-CoV-2 spike protein.

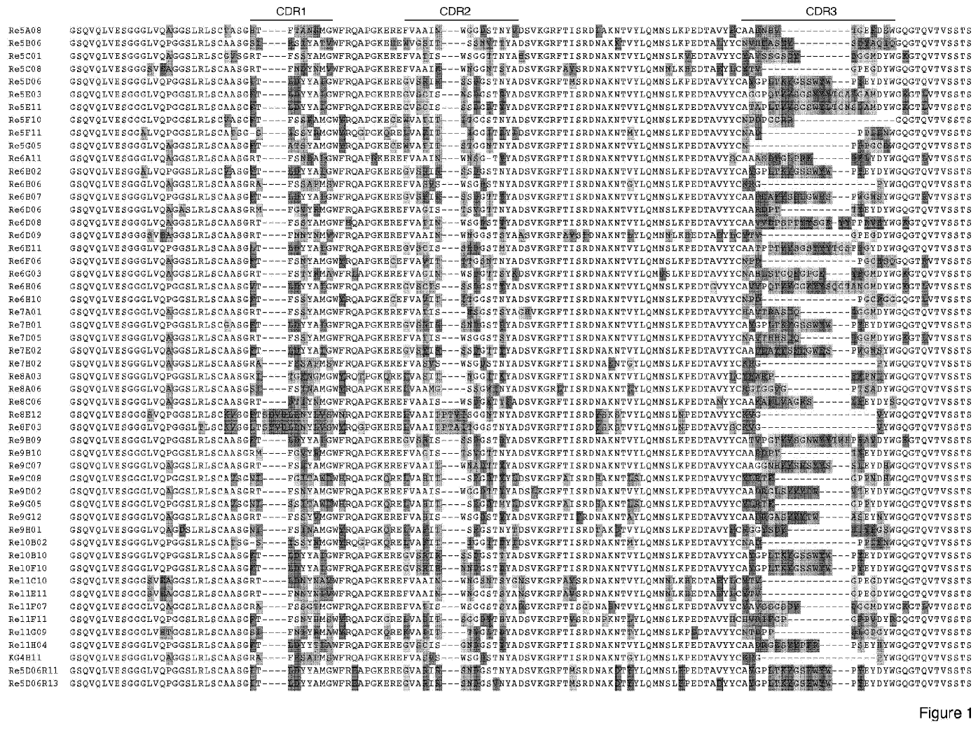

HeLa cells where transfected with a plasmid carrying the humanized coding sequence of the SARS-CoV-2 spike protein under the control of a CMV promoter. 36 hours post-transfection, cells were fixed for 5 minutes with 4% paraformaldehyde (PFA), permeabilized, blocked with 5% BSA, incubated with 30 nM VHH Re6D09 carrying two Alexa568 fluorophores per molecule, extensively washed and finally imaged with an LSM780 confocal laser scanning microscope using the 405 nm and 568 nm laser lines for excitation. Image shows the overlaid DAPI and 568 channels, detecting DNA and VHH antibody in blue and red, respectively. Note that the transfection efficiency was only ˜30%. The bright red signal corresponds to transfected cells, while non-transfected ones served as negative control.

FIG. 3: Fluorophore-labelled anti-SARS-CoV-2 VHH antibodies specifically detect the spike protein and assembling viruses in infected cells.

Vero E6 cells were infected by SARS-CoV-2 for three days, fixed for two hours with 4% paraformaldehyde, and stained as described above with 30 nM of the indicated Alexa488-labelled VHH antibodies. Imaging was with a standard epifluorescence microscope.

FIG. 4: SARS-CoV-2 neutralizing VHH antibodies.

At day 0, Vero E6 cells were inoculated with SARS-CoV-2, in the absence of VHHs or after a 60 min pre-incubation with the indicated VHH antibodies. Three days later, the virus-load increased ˜10 000-fold when the antibodies had been omitted (compare “inoculation” and “no VHH”). One VHH (Re7D02) had no effect. Two (Re7D05 and Re5C08) had a weak impact. Re6B06 inhibited in this experiment ˜300-fold. The other 18 VHH antibodies blocked viral infection completely. Quantitation of viral RNA in the supernatants of infected cells was by quantitative reverse transcription (RT) PCR as described previously (Stegmann et al., 2020). Note the Log 10-scale of the figure.

FIG. 5: VHH Re5D06 neutralizes SARS-CoV-2 with extreme potency.

Virus neutralization was performed as in FIG. 4. (A) Cells were fixed with PFA and stained with DAPI (to visualize cell nuclei), with a cocktail of Atto488-labelled VHHs recognizing the RBD (RBD epitopes) and Atto565-labelled VHHs recognizing an S1 epitope outside the RBD (S1ΔRBD epitope) in order to visualize newly synthesized Spike protein in successfully infected cells. Images show confocal sections. Cells that are positive in the Atto488 and Atto656 channels are infected. Note that infection was completely prevented when the virus was pre-incubated with VHH Re5D06 at a concentration of 50 pM. (B) A replicate experiment with identical outcome. Bars depict analysis by quantitative RT PCR, showing that 50 pM VHH Re5D606 prevented replication of the viral RNA completely.

FIG. 6: Hyper-thermostable anti-SARS-CoV-2 VHH antibodies.

Indicated VHH antibodies were subjected to Differential scanning fluorimetry (DSF), which exploits that thermal unfolding exposed aromatic/hydrophobic residues, which then bind and enhance fluorescence of the added SYPRO orange dye. Assays were performed in a volume of 20 μl, at 1 mg/ml VHH concentration in 50 mM Tris/HCl, 300 mM NaCl (pH 8.0 at 20° C.) and 1× dye (diluted from a 5000× stock; Life Technologies). Three replicates of each sample were pipetted in a Hard-Shell® 96-well plate (Bio-Rad). The plate was sealed with transparent MicroSeal® ‘B’ Seal (Bio-Rad), briefly centrifuged to remove any air bubbles, and placed onto a CFX96 Real-Time System (C1000 Thermal Cycler, BioRad). The samples were incubated for 5 min at 25° C. and then the temperature was increased in 1° C. increments of 45 seconds to 95° C. Fluorescence was measured at the end of each step with 532 nm excitation and a 555 nm long pass filter. Melting temperatures are defined as the inflection point of the first melting peak. Note that the super-neutralizing VHH Re5D06 melts already at 50° C., while the optimized Re5D06R13 version remained fully stable throughout 95° C., as did Re6H06 and Re6B06. Re5D06R13 and Re6B06 retained their hyper-thermostability even in the presence of disulphide-bond reducing DTT.

FIG. 7: Highly potent symmetry matching anti SARS-CoV-2 VHH antibodies.

(A) Scheme of a homotrimeric VHH fusion to match the C3 rotational symmetry of the Spike of SARS-CoV-2 and other viruses. (B) Comparison of neutralization potencies of ReB06 monomers and Re6B06-spacer-Collagen XVIII NC1 trimers. The experiment was performed analogously to FIG. 5; however, overlaid fluorescent channels in extended focus are shown. Note that the trimer neutralizes at a 30,000-fold lower VHH concentration than the monomer. (C) Comparison between a VHH-72 monomer and a VHH-72 trimer. Note that the trimer neutralizes at a 10000-fold lower VHH concentration than the corresponding monomer.

FIG. 8: Trimerization caused a strong avidity effect for the VHH-spike interaction

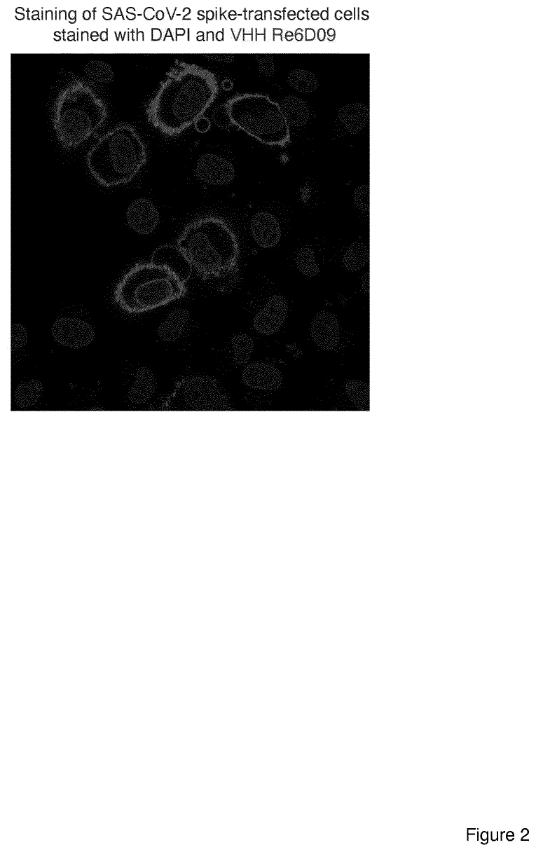

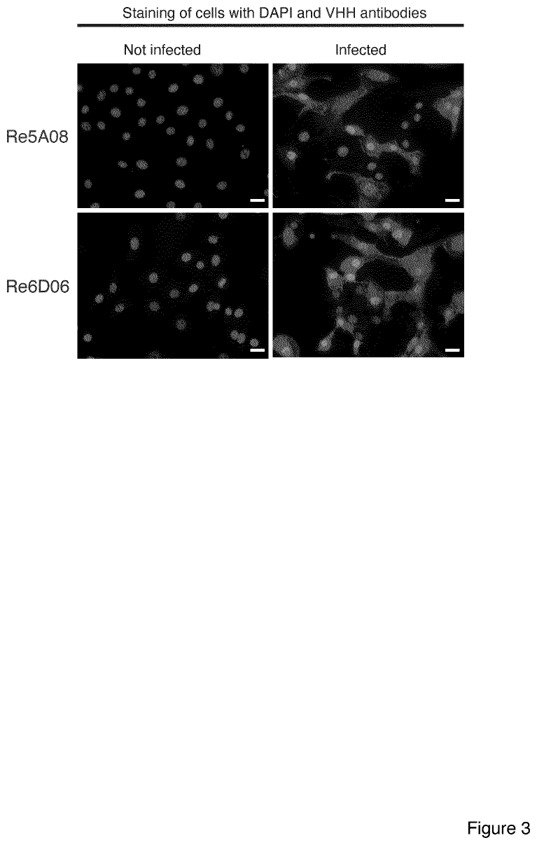

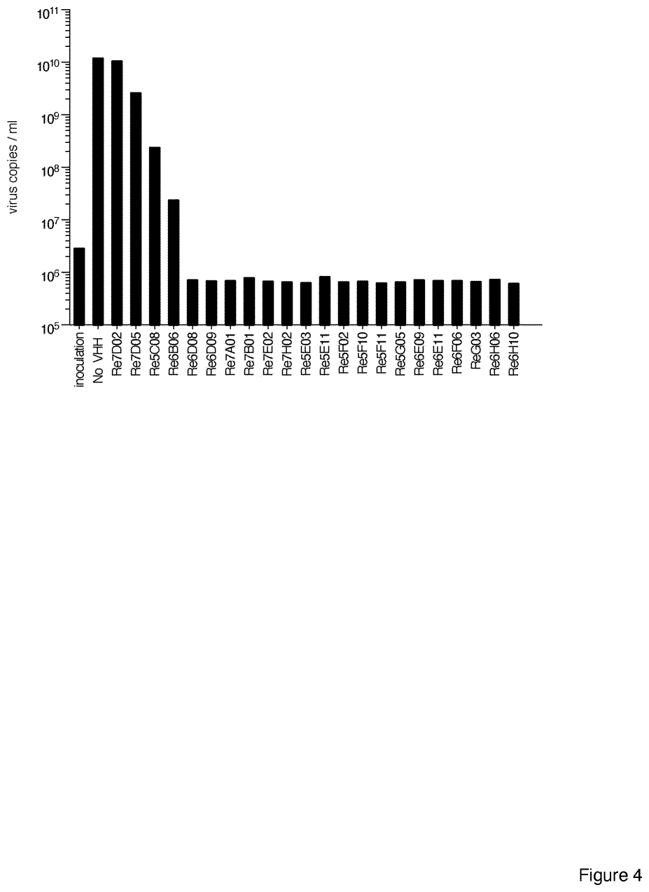

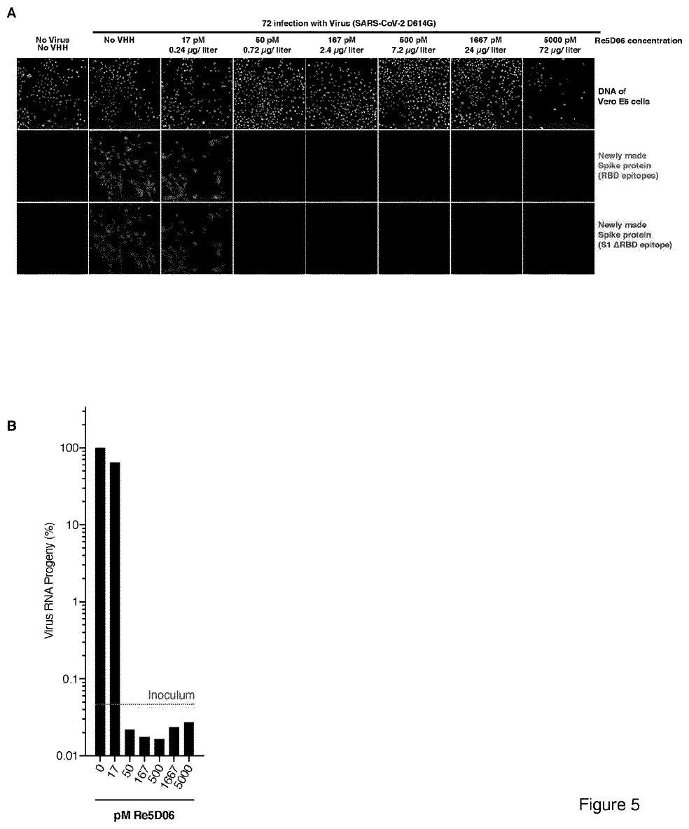

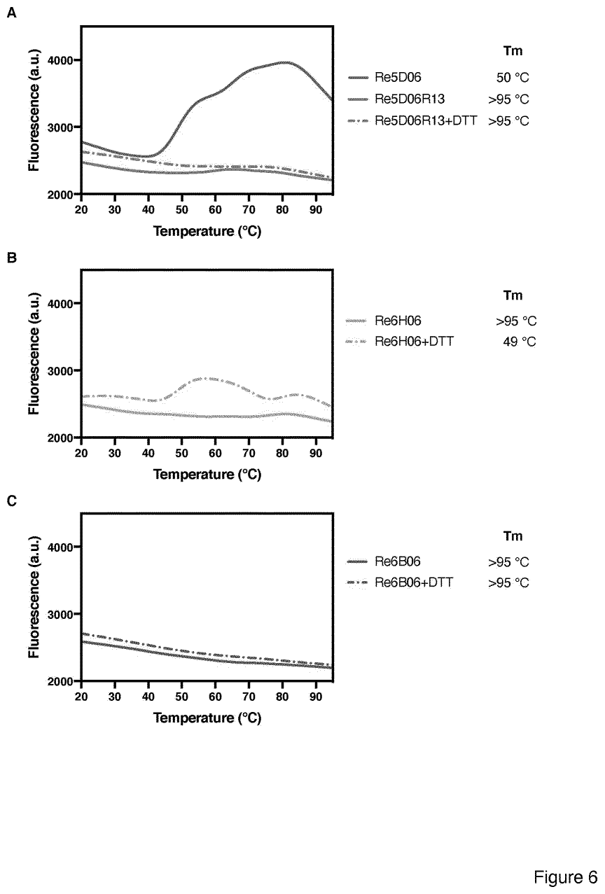

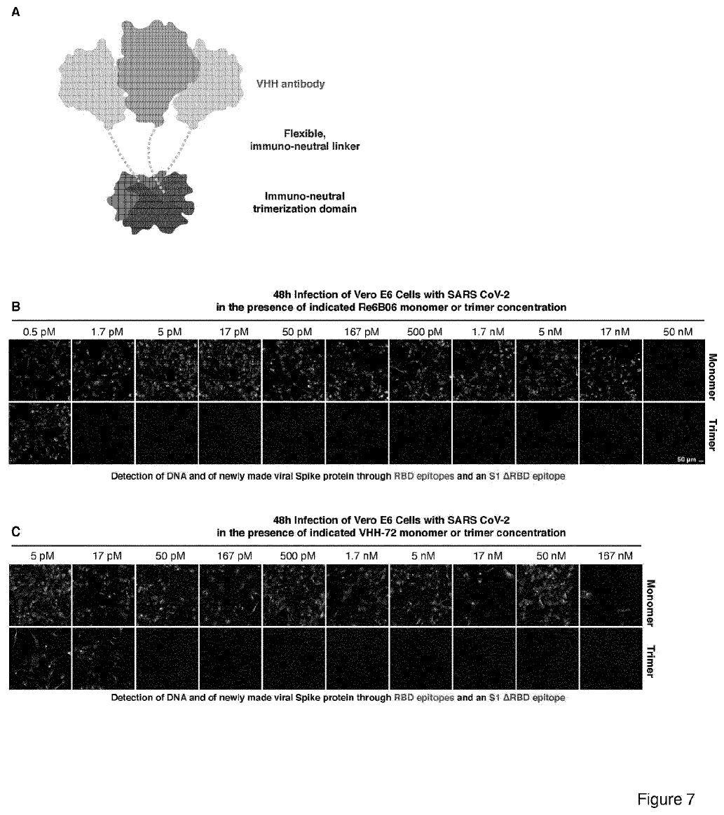

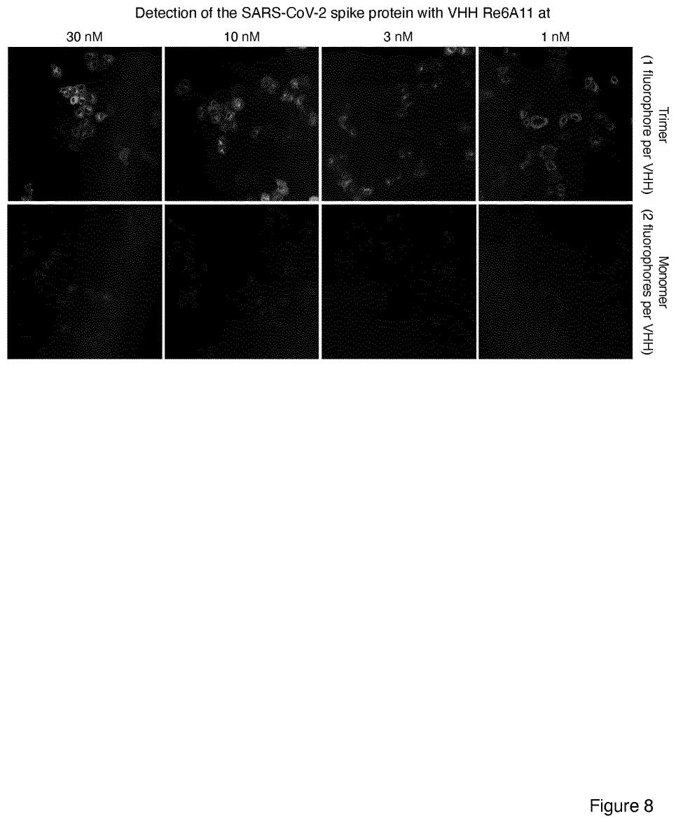

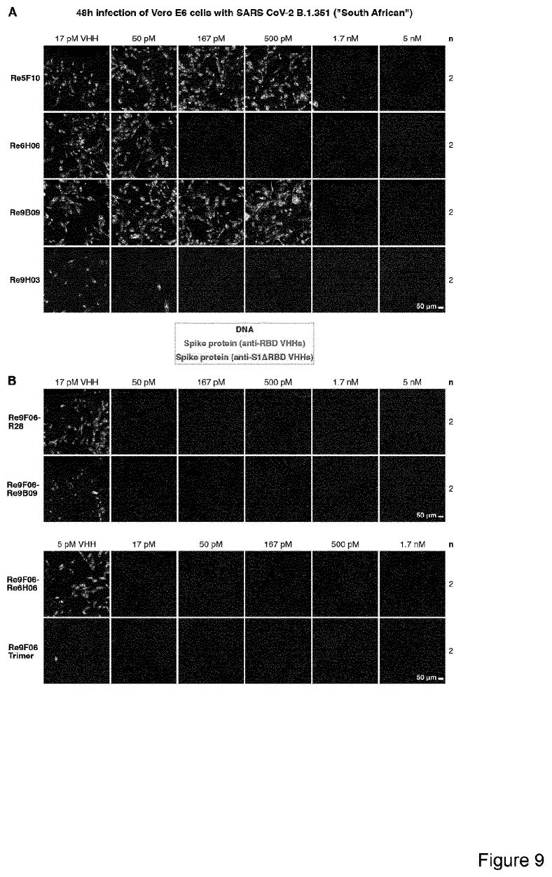

Hela cells were transfected as described in FIG. 2 to transiently express the SARS-Co-V2 spike protein. Following fixation, they were stained with Alexa488-labelled VHH Re6A11. In monomeric form, the staining was very weak even at 30 nM and with 2 fluorophores per VHH. In contrast, staining was strong with the trimerized version, even at a much lower concentration of 1 nM VHH and with only one fluorophore per VHH.

FIG. 9: Neutralization of SARS-CoV-2 B.1.351 by VHH antibodies.

(A) and (B): Neutralization of SARS-CoV-2 B.1.351 by the indicated VHH antibody constructs. The neutralization experiment was performed as described in FIG. 5, with the difference being that a mix of mutant-optimized anti-RBD/S1 VHH antibodies were used for immunofluorescence staining.

VHH antibody sequences
>Re5A08
GSQVQLVESGGGLVQAGGSLRLSCTASGHTFTANRMGWFRQAPGKEREFV
AAINWGGDSTNYVDSVKGRFTISRDIAKNTVYLQMNSLKPEDTAVYFCAA
RNHVTGEFDSWGQGTQVTVSSTS
>Re5B06
GSQVQLVESGGGLVQPGGSLRLSCAASGSIRSIYATVWFRQAPGKEHEWV
GSITSSNVTTYADSVKGRFTISRDNAKKTVYLQMNSLKPEDTALYYCNVH
FASEYSDYAQIQGQGTQVTVSSTS
>Re5C01
GSQVQLVESGGGLVQAGGSLRLSCGVSGRTFSSYAMGWFRQAPGKEREFV
ATISWSGGTTNYAHSVKGRFTISRDNAKNTVYLQMNSLKVEDTAVYYCYA
VSSGSDYDGGMDYWGKGTQVTVSSTS
>Re5C08
GSQVQLVESGGGSVEAGGSLRLSCAASGRTFNDYNMVWFRQAPGKEREFV
AAIKWNGGNTSYADSVKGRFAVSRDNAKNTVYLQMNNLKHEDTAEYLCYT
VGPEGDYWGQGTQVTVSSTS
>Re5D06
GSQVQLVESGGGLVQPGGSLRLSCAASGITLDYYAIGWFRQAPGKEREGV
SRIRSSDGSTNYADSVKGRFTMSRDNAKNTVYLQMNSLKPEDTAVYYCAY
GPLTKYGSSWYWPYEYDYWGQGTQVTVSSTS
>Re5E03
GSQVQLVESGGGLVQPGGSLRLSCAASGFTLDYYAIGWFRQAPGKEREGV
SCISNSDGSTRYADSVKGRFTISRDNAKNTVYLQMNSLKPEDTAVYYCAG
GPQTYYSGSYYYTCAEGAMDYWGKGTLVTVSSTS
>Re5E11
GSQVQLVESGGGLVQPGGSLRLSCAASGFTLDYYAIGWFRQAPGKEREGV
SCISSSDGRTYYADSVKGRFTISRDNAKNTVYLQMNSLKPEDTAVYYCAT
APLTYYSGSWYLTCNSDAMDYWGKGTLVTVSSTS
>Re5F10
GSQVQLVESGGGLVQPGGSLRLSCVASGFTFSSFAMGWYRQAPGKECEWV
ATITITGGSTNYADSVKGRFTISRDNAKNTVYLQMNSLKPEDTAVYYCNP
DPGCRRGQGTQVTVSSTS
>Re5F11
GSQVQLVESGGALVQPGGSLRLSCATSGSISSYRMGWYRQGPGKQRELVA
FITIGGITDYIDSVKGRFTISRDNAKNTMYLQMNSLKPEDTAVYYCNADP
PLFNWGQGTQVTVSSTS
>Re5G05
GSQVQLVESGGGLVQAGGSLRLSCAASGFTATSYAMGWYRQAPGKECEWV
ATITSTGGNTNYADSVKGRFTISRDNAKNTVYLQMNSLKPEDTAVYYCNP
DPGCDWGQGTQVTVSSTS
>Re6A11
GSQVQLVESGGGLVQAGGSLRLSCAASGRTFSNDALGWFRQAPRKEREFV
AAINWNSGTYYADSVKGRFTISRDNAKNTVYLQMNSLKPEDTAVYSCAAA
SDYGLPREDFLYDYWGQGTLVTVSSTS
>Re6B02
GSQVQLVESGGALVQPGGSLRLSCVASGFTLDYYAIGWFRQAPGKEREGV
SRIRSSDGSTNYADSVKGRFTISRDNAKNTVYLQMNSLKPEDTAVYYCAY
GPLTKYGSSWYWPYEYDYWGQGTQVTVSSTS
>Re6B06
GSQVQLVESGGGLVQAGGSLRLSCAASGRAFSSAPMSWFRQAPGKEREFV
ASVSWSGDSTNYADSVKGRFTISRDNAKNTGYLQMNSLKPEDTAVYYCKR
GPYWGQGTQVTVSSTS
>Re6B07
GSQVQLVESGGGLVQPGGSLRLSCAASGFTLDYYAIGWFRQAPGKEREGV
SYIRSSDGTTYYADSVKGRFTISRDNAKNTVYLQMNSLKPEDTAVYYCAA
DEAYYSELGWESPWGWSYWGQGTRVTVSSTS
>Re6D06
GSQVQLVESGGGLVQAGASLRLSCAASGRMFGVYRMGWFRQAPGKEREFV
AGISTSVGTTNYADSVKGRFTISRDNAKNTVYLQMNSLKPEDTAVYYCAA
RDPTTYEYDYWGQGTQVTVSSTS
>Re6D08
GSQVQLVESGGGLVQAGGSLRLSCAASGRTFSSYAMGWFHQAPGKEREFV
ATINWSGDSTYYADSVKGRFTISRDNAKNTVYLQMNSLKPEDTAVYYCAA
VVDPSPTYYSGKYYPPRVEYWGKGTQVTVSSTS
>Re6D09
GSQVQLVESGGGSVEAGGSLRLSCAASGRTFNNYNMVWFRQAPGKEREFV
AAINWNGGSTSYAASVKGRFAVSIDNAKNTLYLQMNNLKHEDTAEYLCYT
VGPEGDYWGQGTQVTVSSTS
>Re6E11
GSQVQLVESGGGLVQPGGSLRLSCAASGLTLDYYAIGWFRQAPGKEREGV
SCISSRDGSTMYADSVKGRFTISRDNAKNTVYLQMNSLKPEDTAVYYCAA
TPTTYYSGSYYYTCSPEGYDYWGQGTQVTVSSTS
>Re6F06
GSQVQLVESGGGLVQAGGSLRLSCAASGFTFSNYAMGWYRQAPGKECEFV
AVITITGSNTNYADSVKGRFTISRDNAKNTVYLQMNSLKPEDTAVYYCNP
DPGCESQGQGTRVTVSSTS
>Re6G03
GSQVQLVESGGGLVQAGGSLRLSCAASGRTFSTYRMAWFRLAPGKEREFV
AGINWSDGTTSYKDSVKGRFTISRDNAKNTVYLQMDSLKPEDTAVYYCNA
HLSTGQEGPGEYFGMDYWGKGTQVTVSSTS
>Re6H06
GSQVQLVESGGGLVQPGGSLRLSCAASGVTLDYYAIGWFRQAPGKEREGV
SCTSSSDGSTYYADSVKGRFTISRDNAKNTVYLQMNSLKPEDTGVYYCAV
VPQTYYGGKYYSQCTANGMDYWGKGTLVTVSSTS
>Re6H10
GSQVQLVESGGGLVQPGGSLRLSCAASGFTFSSYAMGWYRQAPGKECEFV
AVITITGGSTNYADSVKGRFTISRDNAKNTVYLQMNSLKPEDTAVYYCNP
DPGCRGGGQGTLVTVSSTS
>Re7A01
GSQVQLVESGGGLVQAGGSLRLSCAASGRTFSSYAMGWFRQAPGKEREFV
ATISFSGSTSYAGHVKGRFTISRDNAKNTVYLQMNSLKPEDTAVYYCHAV
TRASDQDGGMDYWGQGTQVTVSSTS
>Re7B01
GSQVQLVESGGGLVQPGGSLRLSCGASGFTLDYYAIGWFRQAPGKEREGV
SRIRSNDGSTDYADSVKGRFTISRDNAKNTVYLQMNSLKPEDTAVYYCAY
GPLTKYGSSWYWPYEYDYWGQGTQVTVSSTS
>Re7D05
GSQVQLVESGGGLVQAGGSLRLSCAASGRTFSSYAMGWFRQAPGKEREFV
ATISWSGGSTSYADSVKGRFTISRDNAKNTVYLQMNSLKPEDTAVYYCNA
VTHHSDQDGGMDYWGKGTLVTVSSTS
>Re7E02
GSQVQLVESGGGLVQAGGSLRLSCAASGFTLDYYAIGWFRQAPGKEREGV
SYIRSSDGTTYYADSVKGRFTISRDNAKNTVYLQMNSLKPEDTAVYYCAA
DEAYYSELGWESPWGWSYWGQGTQVTVSSTS
>Re7H02
GSQVQLVESGGGLVQAGGSLRLSCAASGRAFESAPMSWFRQAPGKEREFV
ASVSWSGDSTNYADSVKGRFTISRDNAENTGYLQMNSLKPEDTAVYYCKR
GPYWGQGTQVTVSSTS
>Re8A03
GSQVQLVESGGGLVQPGGSLRLSCAASGRITGFNGMGWYRQTPGKQRELV
ASITNGGITKYADSVKGRFTISRDNAKNTVYLQMNSLKPEDTAVYLCYFW
RPEFPNLYWGQGTQVTVSSTS
>Re8A06
GSQVQLVESGGGLVQAGGSLRLSCAASGSIFSINAMGWYRQAPGKERELV
AAMGSSGWINYADSVKGRLTISRDNAKNTLYLQMNSLKPEDTAVYYCRGT
GGVGPTSADYWGQGTQVTVSSTS
>Re8C06
GSQVQLVESGGGLVQAGGSLRLSCAASGRTDTIYNMGWFRQAPGKEREFV
AAISWSDGKTTFADSVKGRFTISRDNAKNTVYLQMNSLKPEDTANYYCAA
KAFLVAGRSLEEYDYSGQGTQVTVSSTS
>Re8E12
GSQVQLVESGGGSVQPGGSLRLSCKVSGFTSDVDLRNYLVSWNRQAPGKE
RELVAAITPTVISGGNTNYADSVKGRFTISRDYSKSTVYLQMNSLNPEDT
AVYYCKVGVYWGQGTQVTVSSTS
>Re8F03
GSQVQLVESGGGLVQPGGSLTLSCKVSGLTSYVDLRNYLVSWYRQGPGKE
RELVAAITPTAITGGSTNYADSVKGRFTISRDYSKSTVYLQMNSLNPEDT
AVYSCKVGVYWGQGTQVTVSSTS
>Re9B09
GSQVQLVESGGGLVQPGGSLRLSCAASGFTLDYYAIGWFRQAPGKEREGV
SRISSSDGSTDYADSVKGRFTISRDNAKNTVYLQMNSLKPEDTAVYYCAT
VPGTYYSGNWYYTWHPEAVDYWGKGTQVTVSSTS
>Re9B10
GSQVQLVESGGGLVQPGGSLRLSCAASGRMFGVYRMGWFRQAPGKEREFV
AGISTSVGTTNYADSVKGRFTISRDNAKNTVYLQMNSLKPEDTAVYYCAA
RDPTTYEYDYWGQGTQVTVSSTS
>Re9C07
GSQVQLVESGGGLVQAGGSLRLSCAASGRTFSRYAMGWFRQAPGKEREFV
AAITWNADTTYYADSVKGRFTISRDNAKNTVYLQMNSLKPEDTAVYYCAA
GGNHYYSRSYYSSLEYDHWGQGTQVTVSSTS
>Re9C08
GSQVQLVESGGGLVQPGGSLRLSCAVSGNIFGITAWDWHRQAPGKQRELV
AHITSRGDTYYLDSVKGRFAISRDHAKNTLSLQMNSLKPEDTAVYYCYLR
TFGPPNDHWGQGTQVTVSSTS
>Re9D02
GSQVQLVESGGGLVQAGGSLRLSCAASGRTFSNYAMGWFRQAPGKEREFV
AAISWGGDTTYYADSLKGRFTISRDNAKNTVYLQMNSLKPEDTAVYYCAA
DRGLSYYYDRVTEYDYWGQGTQVTVSSTS
>Re9G05
GSQVQLVESGGGLVQPGGSLRLSCAVSGNISSITAWDWHRQAPGKQRELV
AHITSRGDTMYLDSVKGRFAISRDHAKNTLSLQMNSLKPEDTAVYYCYLR
TFGPPYDYWGQGTQVTVSSTS
>Re9G12
GSQVQLVESGGGLVQAGGSLRLSCAASGRTFSSYVMGWFRQAPGKEREFV
AHISWSGDSTYYADSVKGRFTIFRDNAKNTAYLQMNSLKPEDTAVYYCAA
DRGASYYYTWASEYNYWGQGTQVTVSSTS
>Re9H01
GSQVQLVESGGGLVQAGDSLRLSCAASGNIFSINAMGWYRQAPGKQRELV
AFITSRGSTNYTDSVKGRFTISRDTAKDTVYLQMNSLKPEDTAVYFCRGG
YSDYDIYFGSWGQGTQVTVSSTS
>Re10B02
GSQVQLVESGGGLVQPGGSLRLSCATSGSISSYRMGWYRQGPGKQRELVA
FITIGGITDYIDSVKGRFTISRDNAKNTMYLQMNSLKPEDTAVYYCNADP
PLFNWGQGTQVTVSSTS
>Re10B10
GSQVQLVESGGGLVQPGGSLRLSCAASGFTLDYYAIGWFRQAPGKEREGV
SRIRSSDGSTTYADSVKGRFTISRDNAKNTVYLQMNSLKPEDTAVYYCAY
GPLTKYGSSWYWPYEYDYWGQGTQVTVSSTS
>Re10F10
GSQVQLVESGGGLVQPGGSLRLSCAASGFTLDYYAIGWFRQAPGKEREGV
SRIRNNDGSTDYADSVKGRFTISRDNAKNTVYLQMNSLKPEDTAVYYCAY
GPLTKYGSSWYWPYEYDYWGQGTQVTVSSTS
>Re11C10
GSQVQLVESGGGSVEAGGSLRLSCAASGRTLDNYNAVWFRQAPGKEREFV
AAINWNGSNTSYGNSVKGRFAVSRDNAKNTVYLQMNNLKHEDTAEYLCYT
VGPEGDYWGQGTQVTVSSTS
>Re11E11
GSQVQLVESGGGSVEAGGSLRLSCAASGRTFNNYNIVWFRQAPGKEREFV
AAINWNGGSTSYANSVKGRFAVSRDNAKNTVYLQMNNLKHEDTAEYLCYT
VGPEGDYWGQGTQVTVSSTS
>Re11F07
GSQVQLVESGGGLVQAGGSLRLSCAASGRAFSSGTMGWFRQAPGKEREFV
ATISWSGGSTSYARSVKGRFTISGDNAENTVYLQMNSLKPEDTAVYYCYA
VSSGSDYDGGMDYWGKGTLVTVSSTS
>Re11F11
GSQVQLVESGGGLVQPGGSLRLSCAASGFTFSNYHMSWYRQAPGKGRELV
ADITSGGDYTHYADSVKGRFTVSRDNPKNTLYLQMNSLKPEDTAVYHCHV
RIFGPGFPVDYRGQGTQVTVSSTS
>Re11G09
GSQVQLVESGGGLVHTGGSLRLSCAASGSIFNIYRMAWYRQAPGKQREKV
AIITTYGLTDYADSVKGRFTISRDNAKNTTYLQMNSLKPDDTAVYYCNTD
PPDLGPGYWGQGTQVTVSSTS
>Re11H04
GSQVQLVESGGGLVQPGGSLRLSCAASGFTLDYYTIAWFRQAPGKEREGV
SCISGNDGSTYYADSVKGRFTISRDNAKNTVYLQMNSLKPEDTAVYYCAA
DRGESYYPFRPSEYHYWGQGTQVTVSSTS
>KG4B11
GSQVQLVESGGGLVQAGGSLRLSCAASGRAFESAPMSWFRQAPGKEREFV
ASVSWSGDSTNYADSVKGRFTISRDNAKNTGYLQMNSLKPEDTAVYYCKR
GPYWGQGTQVTVSSTS
>Re5D06R11
GSQVQLVESGGGLVQPGGSLRLSCAASGITLDYYAMGWFREAPGKEREGV
ARIRSNDGSTNYADSVKGRFTMSRDNAKDTYYLQMNSLEPEDTAVYYCAY
GPLTKYGSEWYWPYEYDYWGQGTQVTVSSTS
>Re5D06R13
GSQVQLVESGGGLVQPGGSLRLSCAASGITLDYYAMGWFREAPGKEREGV
ARIRSNDGSVNYADSVKGRFTMSRDNAKDTYYLQMNSLEPEDTADYYCAY
GPLTKYGSEWYWPYEYDYWGQGTQVTVSSTS
>Re5D06R15
GSQVQLVESGGGLVQPGGSLRLSCAASGITLDYYAMGWFREAPGKEREGV
ARIRNSDGSTNYADSVKGRFTMSRDNAKDTYYLQMNSLEPEDTADYYCAY
GPLTKYGSSWYWPYEYDYWGQGTQVTVSSTS
>Re5D06R23
GSQVQLVESGGGLVQPGGSLRLSCAISGSTLDYYAMGWFREAPGKEREGV
ARIRNNDGSTDYADSVKGRFTMSRDNAKDTYYLQMNSLEPEDTADYYCAY
GPLTKYGSSWYWPYEYDYWGQGTQVTVSSTS
>Re5D06R28
>GSQVQLVESGGGLVQPGGSLRLSCAISGSTLDYYAMGWFREAPGKEREG
VARWRNNDGSTNYADSVKGRFTMSRDNAKDTYYLQMNSLEPEDTADYYCA
YGPLTKYGSSWHWPYEYDYWGQGTQVTVSSTS
>Re5D06R28D
GSQVQLVESGGGLVQPGGSLRLSCAISGSTLDYYAMGWFREAPGKEREGV
ARWRNNDGSTNYADSVKGRFTMSRDNAKDTYYLQMNSLEPEDTADYYCAY
GPLTKYGDEWHWPYEYDYWGQGTQVTVSSTS
>Re5D06R15_3QE
GSQVQLVESGGGLVEPGGSLRLSCAASGITLDYYAMGWFREAPGKEREGV
ARIRNSDGSTNYADSVKGRFTMSRDNAKDTYYLQMNSLEPEDTADYYCAY
GPLTKYGSSWYWPYEYDYWGEGTEVTVSSTS
>Re5D06R28_3QE
GSQVQLVESGGGLVEPGGSLRLSCAISGSTLDYYAMGWFREAPGKEREGV
ARWRNNDGSTNYADSVKGRFTMSRDNAKDTYYLQMNSLEPEDTADYYCAY
GPLTKYGSSWHWPYEYDYWGEGTEVTVSSTS
>Re9F06
GSQVQLVESGGGLVQAGGSLRLSCAASGRTFSNDALGWFRQAPRKEREFV
AAINWNSGTYYADSVKGRFTISRDNAKNTVYLQMNSLKPEDTAVYSCAAA
SDYGLPREDFLYDYWGQGTQVTVSSTS
>Re9H03
GSQVQLVESGGGLVQPGGSLRLSCAASGFTLDYYAIGWFRQAPGKEREGV
SRISSSDGSTDYADSVKGRFTISRDNAKNTVYLQMNSLKPEDTAVYYCAT
VPGTYYSGNWYYTWHPKAVDYWGKGTLVTVSSTS
>Re21B09
GSQVQLVESGGGLVQPGGSLRLSCAASGFTLDNYAIGWFRQAPGKEREGV
SCIRSSDGSTYYADSVKGRFTISKDNAKNTVYLQMNSLKPEDTAVYYCAT
DGTFNPPCDDLYSWYFPERQGTQVTVSSTS
>Re21D01
GSQVQLVESGGALVQPGGSLRLSCVASGFTFSSFAMGWYRQAPGKECEWV
ATITITGGSTNYADSVKGRFTISRDNAKNTVYLQMNSLKPEDTAVYYCNP
DPGCRRGQGTQVTVSSTS
>Re21H01
GSQVQLVESGGALVQPGGSLRLSCVASGFTFSSFAMGWYRQAPGKECEWV
ATITITGGSTNYADSVKGRFTISRDNAKNTLYLQMNSLKPEDTAVYYCNP
DPGCRGGGQGTQVTVSSTS
>Re22D04
GSQVQLVESGGGLVQTGGSLRLSCAASGRTFSDDAMGWFRQAPGKERDVV
AALGWAGVSTYYADSVKGRFGISRDNAKNTVYLQMSSLKPEDTAVYYCAA
APSVAHARLGEWAYWGKGTQVTVSSTS
>Re22E05
GSQVQLVESGGGLVQPGGSLRLSCAASGFTLDYYAIGWFRQAPGKEREGV
SRISSSDGSTDYADSVKGRFTISRDNAKNTVYLQMNSLKPEDTAVYYCAT
VPGTYYSGNWYYTWHPKAVDYWGKGTQVTVSSTS
>Re25H10
GSQVQLVESGGGLVQPGGSLRLSCAASGFTFSSSAMSWARQAPGKGLEWV
STISEDGSTYYADSMKGRFTISRDNAKNTVYLQMNSLKPEDTAAYYCATS
TEPRTVVAGWGDYLGQGTQVTVSSTS
>Re26D07
GSQVQLVESGGGLVQPGGSLRLSCAASGVTLDYYAIGWFRQAPGKEREGV
SCTSSSDGSTYYADSVKGRFTISRDNAKNTVYLQMNSLKPEDTGVYYCAV
VPQTYYGGKYYSQCTANGMDYWGKGTQVTVSSTS
>Re26E09
GSQVQLVESGGALVQPGGSLRLSCVASGFTFSSFAMGWYRQAPGKECEWV
ATITITGGNTNYADSVKGRFTISRDNAKNTVYLQMNSLKPEDTAVYYCNP
DPGCRRGQGTRVTVSSTS
>Re26E11
GSQVQLVESGGALVQPGGSLRLSCVASGFTFSSFAMGWYRQAPGKECEWV
ATITITGGNTNYADSVKGRFTISRDNAKNTVYLQMNSLKPEDTAVYYCNP
DPGCRRGQGTQVTVSSTS
>VHH-72 monomer
GSGQVQLQESGGGLVQAGGSLRLSCAASGRTFSEYAMGWFRQAPGKEREF
VATISWSGGSTYYTDSVKGRFTISRDNAKNTVYLQMNSLKPDDTAVYYCA
AAGLGTVVSEWDYDYDYWGQGTQVTVSSGS

Sequences of heterodimeric VHH antibodies
>Re9F06-SpacerA-Re9B09|pDG03599
(SEQ. ID NO: 292)
GSQVQLVESGGGLVQAGGSLRLSCAASGRTFSNDALGWFRQAPRKEREFV
AAINWNSGTYYADSVKGRFTISRDNAKNTVYLQMNSLKPEDTAVYSCAAA
SDYGLPREDFLYDYWGQGTQVTVSSTSGEGEGGEGGEGSQVQLVESGGGL
VQPGGSLRLSCAASGFTLDYYAIGWFRQAPGKEREGVSRISSSDGSTDYA
DSVKGRFTISRDNAKNTVYLQMNSLKPEDTAVYYCATVPGTYYSGNWYYT
WHPEAVDYWGKGTQVTVSSTS
>Re9F06-SpacerA-Re5D06R28D|pDG03560
(SEQ. ID NO: 293)
GSQVQLVESGGGLVQAGGSLRLSCAASGRTFSNDALGWFRQAPRKEREFV
AAINWNSGTYYADSVKGRFTISRDNAKNTVYLQMNSLKPEDTAVYSCAAA
SDYGLPREDFLYDYWGQGTQVTVSSTSGEGEGGEGGEGSQVQLVESGGGL
VQPGGSLRLSCAISGSTLDYYAMGWFREAPGKEREGVARWRNNDGSTNYA
DSVKGRFTMSRDNAKDTYYLQMNSLEPEDTADYYCAYGPLTKYGDEWHWP
YEYDYWGQGTQVTVSSTS
>Re22D04-SpacerA-Re5D06R28D|pD0G3661
(SEQ. ID NO: 294)
GSQVQLVESGGGLVQTGGSLRLSCAASGRTFSDDAMGWFRQAPGKERDVV
AALGWAGVSTYYADSVKGRFGISRDNAKNTVYLQMSSLKPEDTAVYYCAA
APSVAHARLGEWAYWGKGTQVTVSSTSGEGEGGEGGEGSQVQLVESGGGL
VQPGGSLRLSCAISGSTLDYYAMGWFREAPGKEREGVARWRNNDGSTNYA
DSVKGRFTMSRDNAKDTYYLQMNSLEPEDTADYYCAYGPLTKYGDEWHWP
YEYDYWGQGTQVTVSSTS
>Re25H10-SpacerA-Re9B09|pDG03697
(SEQ. ID NO: 295)
GSQVQLVESGGGLVQPGGSLRLSCAASGFTFSSSAMSWARQAPGKGLEWV
STISEDGSTYYADSMKGRFTISRDNAKNTVYLQMNSLKPEDTAAYYCATS
TEPRTVVAGWGDYLGQGTQVTVSSTSGEGEGGEGGEGSQVQLVESGGGLV
QPGGSLRLSCAASGFTLDYYAIGWFRQAPGKEREGVSRISSSDGSTDYAD
SVKGRFTISRDNAKNTVYLQMNSLKPEDTAVYYCATVPGTYYSGNWYYTW
HPEAVDYWGKGTQVTVSSTS
>Re25H10-SpacerA-Re5D06R28D|pDG03798
(SEQ. ID NO: 296)
GSQVQLVESGGGLVQPGGSLRLSCAASGFTFSSSAMSWARQAPGKGLEWV
STISEDGSTYYADSMKGRFTISRDNAKNTVYLQMNSLKPEDTAAYYCATS
TEPRTVVAGWGDYLGQGTQVTVSSTSGEGEGGEGGEGSQVQLVESGGGLV
QPGGSLRLSCAISGSTLDYYAMGWFREAPGKEREGVARWRNNDGSTNYAD
SVKGRFTMSRDNAKDTYYLQMNSLEPEDTADYYCAYGPLTKYGDEWHWPY
EYDYWGQGTQVTVSSTS
>Pp086|Re9F06-SpacerB-Re5D06R28D|pDG03637
(SEQ. ID NO: 297)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGEGSQVQLVESGG
GLVQPGGSLRLSCAISGSTLDYYAMGWFREAPGKEREGVARWRNNDGSTN
YADSVKGRFTMSRDNAKDTYYLQMNSLEPEDTADYYCAYGPLTKYGDEWH
WPYEYDYWGQGTQVTVSS
>Pp087|Re9F06-SpacerC-Re6H06|pDG03625
(SEQ. ID NO: 298)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGSGEGGSEGGEGG
SGEGSQVQLVESGGGLVQPGGSLRLSCAASGVTLDYYAIGWFRQAPGKER
EGVSCTSSSDGSTYYADSVKGRFTISRDNAKNTVYLQMNSLKPEDTGVYY
CAVVPQTYYGGKYYSQCTANGMDYWGKGTLVTVSS
>Pp088|Re9F06-SpacerC-Re9B09|pDG03626
(SEQ. ID NO: 299)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGSGEGGSEGGEGG
SGEGSQVQLVESGGGLVQPGGSLRLSCAASGFTLDYYAIGWFRQAPGKER
EGVSRISSSDGSTDYADSVKGRFTISRDNAKNTVYLQMNSLKPEDTAVYY
CATVPGTYYSGNWYYTWHPEAVDYWGKGTQVTVSS
>Pp089|Re9F06-SpacerB-Re6H06|pDG03627
(SEQ. ID NO: 300)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGEGSQVQLVESGG
GLVQPGGSLRLSCAASGVTLDYYAIGWFRQAPGKEREGVSCTSSSDGSTY
YADSVKGRFTISRDNAKNTVYLQMNSLKPEDTGVYYCAVVPQTYYGGKYY
SQCTANGMDYWGKGTLVTVSS
>Pp090|Re9F06-SpacerB-Re9B09|pDG03628
(SEQ. ID NO: 301)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGEGSQVQLVESGG
GLVQPGGSLRLSCAASGFTLDYYAIGWFRQAPGKEREGVSRISSSDGSTD
YADSVKGRFTISRDNAKNTVYLQMNSLKPEDTAVYYCATVPGTYYSGNWY
YTWHPEAVDYWGKGTQVTVSS
>Pp091|Re9F06-SpacerB-Re5D06R15_3QE|pDG03629
(SEQ. ID NO: 302)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGEGSQVQLVESGG
GLVEPGGSLRLSCAASGITLDYYAMGWFREAPGKEREGVARIRNSDGSTN
YADSVKGRFTMSRDNAKDTYYLQMNSLEPEDTADYYCAYGPLTKYGSSWY
WPYEYDYWGEGTEVTVSS
>Pp092|Re9F06-SpacerB-Re5D06R28_3QE|pDG03630
(SEQ. ID NO: 303)
EGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAA
ASDYGLPREDFLYDYWGQGTQVTVSSTSGEGEGGGEGGEGSQVQLVESGG
GLVEPGGSLRLSCAISGSTLDYYAMGWFREAPGKEREGVARWRNNDGSTN
YADSVKGRFTMSRDNAKDTYYLQMNSLEPEDTADYYCAYGPLTKYGSSWH
WPYEYDYWGEGTEVTVSS
>Pp093|Re21D01-SpacerB-Re5D06R15_3QE|pDG03663
(SEQ. ID NO: 304)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGEGSQVQLVESGGGLVEPGGSL
RLSCAASGITLDYYAMGWFREAPGKEREGVARIRNSDGSTNYADSVKGRF
TMSRDNAKDTYYLQMNSLEPEDTADYYCAYGPLTKYGSSWYWPYEYDYWG
EGTEVTVSS
>Pp094|Re21D01-SpacerB-Re5D06R28_3QE|pDG03664
(SEQ. ID NO: 305)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGEGSQVQLVESGGGLVEPGGSL
RLSCAISGSTLDYYAMGWFREAPGKEREGVARWRNNDGSTNYADSVKGRF
TMSRDNAKDTYYLQMNSLEPEDTADYYCAYGPLTKYGSSWHWPYEYDYWG
EGTEVTVSS
>Pp095|Re21D01-SpacerC-Re5D06R15_3QE|pDG03665
(SEQ. ID NO: 306)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGSGEGGSEGGEGGSGEGSQVQL
VESGGGLVEPGGSLRLSCAASGITLDYYAMGWFREAPGKEREGVARIRNS
DGSTNYADSVKGRFTMSRDNAKDTYYLQMNSLEPEDTADYYCAYGPLTKY
GSSWYWPYEYDYWGEGTEVTVSS
>Pp096|Re21D01-SpacerC-Re5D06R28_3QE|pDG03666
(SEQ. ID NO: 307)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGSGEGGSEGGEGGSGEGSQVQL
VESGGGLVEPGGSLRLSCAISGSTLDYYAMGWFREAPGKEREGVARWRNN
DGSTNYADSVKGRFTMSRDNAKDTYYLQMNSLEPEDTADYYCAYGPLTKY
GSSWHWPYEYDYWGEGTEVTVSS
>Pp097|Re21D01-SpacerB-Re6H06|pDG03667
(SEQ. ID NO: 308)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGEGSQVQLVESGGGLVQPGGSL
RLSCAASGVTLDYYAIGWFRQAPGKEREGVSCTSSSDGSTYYADSVKGRF
TISRDNAKNTVYLQMNSLKPEDTGVYYCAVVPQTYYGGKYYSQCTANGMD
YWGKGTLVTVSS
>Pp098|Re21D01-SpacerB-Re9B09|pDG03668
(SEQ. ID NO: 309)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGEGSQVQLVESGGGLVQPGGSL
RLSCAASGFTLDYYAIGWFRQAPGKEREGVSRISSSDGSTDYADSVKGRF
TISRDNAKNTVYLQMNSLKPEDTAVYYCATVPGTYYSGNWYYTWHPEAVD
YWGKGTQVTVSS
>Pp099|Re21D01-SpacerC-Re6H06|pDG03669
(SEQ. ID NO: 310)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGSGEGGSEGGEGGSGEGSQVQL
VESGGGLVQPGGSLRLSCAASGVTLDYYAIGWFRQAPGKEREGVSCTSSS
DGSTYYADSVKGRFTISRDNAKNTVYLQMNSLKPEDTGVYYCAVVPQTYY
GGKYYSQCTANGMDYWGKGTLVTVSS
>Pp100|Re21D01-SpacerC-Re9B09|pDG03670
(SEQ. ID NO: 311)
EGSQVQLVESGGALVQPGGSLRLSCVASGFTFSSFAMGWYRQAPGKECEW
VATITITGGSTNYADSVKGRFTISRDNAKNTVYLQMNSLKPEDTAVYYCN
PDPGCRRGQGTQVTVSSTSGEGEGGGEGGSGEGGSEGGEGGSGEGSQVQL
VESGGGLVQPGGSLRLSCAASGFTLDYYAIGWFRQAPGKEREGVSRISSS
DGSTDYADSVKGRFTISRDNAKNTVYLQMNSLKPEDTAVYYCATVPGTYY
SGNWYYTWHPEAVDYWGKGTQVTVSS

Sequences of subunits for trimeric VHH-spacer
Collagen XVIII NC1 or collagen XV NC1 fusions
expressed in E. coli
>Re6B06 ColXVIII trimer
(SEQ. ID NO: 211)
GSQVQLVESGGGLVQAGGSLRLSCAASGRAFSSAPMSWFRQAPGKEREFV
ASVSWSGDSTNYADSVKGRFTISRDNAKNTGYLQMNSLKPEDTAVYYCKR
GPYWGQGTQVTVSSTSEGSEGPESSDGSDSTDPGEQGEGADASDGSEGAS
SGVRLWATRQAMLGQVHEVPEGWLIFVAEQEELYVRVQNGFRKVQLEART
PLPR
>Re7H02 ColXVIII trimer
(SEQ. ID NO: 212)
GSQVQLVESGGGLVQAGGSLRLSCAASGRAFESAPMSWFRQAPGKEREFV
ASVSWSGDSTNYADSVKGRFTISRDNAENTGYLQMNSLKPEDTAVYYCKR
GPYWGQGTQVTVSSTSEGSEGPESSDGSDSTDPGEQGEGADASDGSEGAS
SGVRLWATRQAMLGQVHEVPEGWLIFVAEQEELYVRVQNGFRKVQLEART
PLPR
>KG4B11 ColXVIII trimer
(SEQ. ID NO: 213)
GSQVQLVESGGGLVQAGGSLRLSCAASGRAFESAPMSWFRQAPGKEREFV
ASVSWSGDSTNYADSVKGRFTISRDNAKNTGYLQMNSLKPEDTAVYYCKR
GPYWGQGTQVTVSSTSEGSEGPESSDGSDSTDPGEQGEGADASDGSEGAS
SGVRLWATRQAMLGQVHEVPEGWLIFVAEQEELYVRVQNGFRKVQLEART
PLPR
>Re6D06 ColXVIII trimer
(SEQ. ID NO: 214)
GSQVQLVESGGGLVQAGASLRLSCAASGRMFGVYRMGWFRQAPGKEREFV
AGISTSVGTTNYADSVKGRFTISRDNAKNTVYLQMNSLKPEDTAVYYCAA
RDPTTYEYDYWGQGTQVTVSSTSEGSEGPESSDGSDSTDPGEQGEGADAS
DGSEGASSGVRLWATRQAMLGQVHEVPEGWLIFVAEQEELYVRVQNGFRK
VQLEARTPLPR
>Re6A11/Re9F06 ColXVIII trimer
(SEQ. ID NO: 215)
GSQVQLVESGGGLVQAGGSLRLSCAASGRTFSNDALGWFRQAPRKEREFV
AAINWNSGTYYADSVKGRFTISRDNAKNTVYLQMNSLKPEDTAVYSCAAA
SDYGLPREDFLYDYWGQGTLVTVSSTSEGSEGPESSDGSDSTDPGEQGEG
ADASDGSEGASSGVRLWATRQAMLGQVHEVPEGWLIFVAEQEELYVRVQN
GFRKVQLEARTPLPR
>Re5A08 ColXVIII trimer
(SEQ. ID NO: 216)
GSQVQLVESGGGLVQAGGSLRLSCTASGHTFTANRMGWFRQAPGKEREFV
AAINWGGDSTNYVDSVKGRFTISRDIAKNTVYLQMNSLKPEDTAVYFCAA
RNHVTGEFDSWGQGTQVTVSSTSEGSEGPESSDGSDSTDPGEQGEGADAS
DGSEGASSGVRLWATRQAMLGQVHEVPEGWLIFVAEQEELYVRVQNGFRK
VQLEARTPLPR
>VHH-72 ColXVIII trimer
(SEQ. ID NO: 218)
GSGQVQLQESGGGLVQAGGSLRLSCAASGRTFSEYAMGWFRQAPGKEREF
VATISWSGGSTYYTDSVKGRFTISRDNAKNTVYLQMNSLKPDDTAVYYCA
AAGLGTVVSEWDYDYDYWGQGTQVTVSSGSGSEGPESSDGSDSTDPGEQG
EGADASDGSEGASSGVRLWATRQAMLGQVHEVPEGWLIFVAEQEELYVRV
QNGFRKVQLEARTPLPR
>ColXV-Re9B09 ColXV trimer
(SEQ. ID NO: 315)
GSEGNLVTAFSNMDDMLQKAHLVIEGTFIYLRDSTEFFIRVRDGWKKLQL
GELIPIPAGSEGPESSDGSDSTDPGEQGEGADASDGSEGGSQVQLVESGG
GLVQPGGSLRLSCAASGFTLDYYAIGWFRQAPGKEREGVSRISSSDGSTD
YADSVKGRFTISRDNAKNTVYLQMNSLKPEDTAVYYCATVPGTYYSGNWY
YTWHPEAVDYWGKGTQVTVSSTS
>Re9B09-ColXVIII trimer
(SEQ. ID NO: 316)
GSQVQLVESGGGLVQPGGSLRLSCAASGFTLDYYAIGWFRQAPGKEREGV
SRISSSDGSTDYADSVKGRFTISRDNAKNTVYLQMNSLKPEDTAVYYCAT
VPGTYYSGNWYYTWHPEAVDYWGKGTQVTVSSTSEGSEGPESSDGSDSTD
PGEQGEGADASDGSEGASSGVRLWATRQAMLGQVHEVPEGWLIFVAEQEE
LYVRVQNGFRKVQLEARTPLPR
>ColXV-Re9H01 trimer
(SEQ. ID NO: 317)
GSEGNLVTAFSNMDDMLQKAHLVIEGTFIYLRDSTEFFIRVRDGWKKLQL
GELIPIPAGSEGPESSDGSDSTDPGEQGEGADASDGSEGGSQVQLVESGG
GLVQAGDSLRLSCAASGNIFSINAMGWYRQAPGKQRELVAFITSRGSTNY
TDSVKGRFTISRDTAKDTVYLQMNSLKPEDTAVYFCRGGYSDYDIYFGSW
GQGTQVTVSST
>Re9H01-ColXVIII trimer
(SEQ. ID NO: 318)
GSQVQLVESGGGLVQAGDSLRLSCAASGNIFSINAMGWYRQAPGKQRELV
AFITSRGSTNYTDSVKGRFTISRDTAKDTVYLQMNSLKPEDTAVYFCRGG
YSDYDIYFGSWGQGTQVTVSSTSEGSEGPESSDGSDSTDPGEQGEGADAS
DGSEGASSGVRLWATRQAMLGQVHEVPEGWLIFVAEQEELYVRVQNGFRK
VQLEARTPLPR
>ColXV-Re7H02 trimer
(SEQ. ID NO: 319)
GSEGNLVTAFSNMDDMLQKAHLVIEGTFIYLRDSTEFFIRVRDGWKKLQL
GELIPIPAGSEGPESSDGSDSTDPGEQGEGADASDGSEGGSQVQLVESGG
GLVQAGGSLRLSCAASGRAFESAPMSWFRQAPGKEREFVASVSWSGDSTN
YADSVKGRFTISRDNAENTGYLQMNSLKPEDTAVYYCKRGPYWGQGTQVT
VSSTS
>KG4B11-ColXV trimer
(SEQ. ID NO: 320)
GSQVQLVESGGGLVQAGGSLRLSCAASGRAFESAPMSWFRQAPGKEREFV
ASVSWSGDSTNYADSVKGRFTISRDNAKNTGYLQMNSLKPEDTAVYYCKR
GPYWGQGTQVTVSSTSEGSEGPESSDGSDSTDPGEQGEGADASDGSEGNL
VTAFSNMDDMLQKAHLVIEGTFIYLRDSTEFFIRVRDGWKKLQLGELIPI
PAD

| | Ost1 signal peptides |
|---|---|
| | >S. cerevisiae Ost1 signal peptide |
| (SEQ. ID NO: 219) | |
| | MRQVWFSWIVGLFLCFFNVSSAA |
| | >P. pastoris Ost1 signal peptide |
| (SEQ. ID NO: 220) | |
| | MKFISILFLLIGSVFG |
| | >S. pombe Ost1 signal peptide |
| (SEQ. ID NO: 221) | |
| | MLVLKLLLWSIISGLSLAE |
| | >C. albicans Ost1 signal peptide |
| (SEQ. ID NO: 222) | |
| | MWKFFITLGVIFSICSA |

Propeptide sequence
>S. cerevisiae propeptide alpha factor
(SEQ. ID NO: 223)
APVNTTTEDETAQIPAEAVIGYSDLEGDFDVAVLPFSNSTNNGLLFINTT
IASIAAKEEGVSLEKR

### Examples

### Example 1—Periplasmic Expression and Purification of VHH Antibody Monomers

VHH antibody Re6H06 was expressed with an N-terminal pelB signal sequence and a C-terminal His10 tag from a Kan-ColE1 plasmid harboring a T5/lac promoter in E. coli NEBExpress (New England Biolabs). A 125-ml pre-culture in Terrific Broth (TB) containing 50 μg/ml Kanamycin was grown overnight at 28° C. to early stationary phase. The culture was then diluted with fresh medium (500 ml, pre-warmed to 37° C.). After 30 minutes of growth at 37° C., protein expression was induced with 0.05 mM IPTG and growth was continued for 2 hours at 37° C., whereby the culture reached a final OD₆₀₀ of ˜8. Bacteria were harvested by centrifugation and lysed by osmotic shock lysis: cell pellets were resuspended in 14 ml 130 mM Tris/HCl pH 8.0, 10 mM EDTA, and sucrose was immediately added to 20% (w/v). After gentle mixing at 23° C. for 30 min, four volumes of ice-cold water were added and mixing was continued at 4° C. for 30 min. 20 mM Tris/HCl pH 7.5, 50 mM NaCl and 20 mM imidazole were added to the cell suspension. Periplasmic extract was then recovered as the supernatant of two consecutive centrifugation steps at 4° C.: a low-speed spin at 4000×g (20 min, F13 rotor, Thermo Fisher Scientific) and a high-speed spin at 38000 rpm (˜1 hour, T647.5 rotor). The VHH antibody was purified at 4° C. via Ni²⁺ EDTA-amide chelate affinity chromatography (1 ml matrix). Beads were washed with ten column volumes of 50 mM Tris/HCl pH 7.5, 300 mM NaCl, 20 mM imidazole, 0.2% (w/v) Triton X-100 and ten column volumes of buffer lacking detergent. After elution with 50 mM Tris/HCl pH 7.5, 300 mM NaCl, 500 mM imidazole, the buffer was exchanged to 50 mM Tris/HCl pH 7.5, 300 mM NaCl, 250 mM sucrose via a PD 10 desalting column (GE Healthcare). Aliquots were frozen in liquid nitrogen and stored at −80° C. This expression/purification strategy was used for all VHH antibodies containing four cysteines and thus two disulfide bonds (Re5E03, Re5E11, Re5G05, Re6E11, Re6F06, Re6G03, Re6H06, Re6H10) as well as for Re5F11.

### Example 2—Cytoplasmic Expression and Purification of VHH Monomers

All other VHH antibodies comprising just two cysteines and thus a single disulfide bond were produced as His14-ScSUMO fusions by cytoplasmic expression in E. coli NEBExpress Shuffle, which allows forming disulfide bonds in the bacterial cytoplasm. In brief, 125 ml pre-cultures were grown overnight at 35° C. in TB+50 μg/ml kanamycin in 5 liter flasks to early stationary phase. They were then diluted with 250 ml fresh medium, shifted to 21° C. and induced for 5 hours with 0.08 mM IPTG. 5 mM EDTA was added, bacteria were pelleted, resuspended in 50 mM Tris/HCl pH 7.5, 20 mM imidazole/HCl pH 7.5, 300 mM NaCl, frozen in liquid nitrogen and lysed by thawing plus sonication. Insoluble material was removed by ultracentrifugation at 38000 rpm (˜1 hour, T647.5 rotor).

The supernatant was applied to a 1 ml Ni²⁺ EDTA-amide chelate column. The matrix was sequentially washed in resuspension buffer, resuspension buffer+0.2% TritonX100, resuspension buffer+700 mM NaCl, low salt buffer (resuspension buffer minus NaCl), and protease buffer (resuspension buffer with an imidazole concentration lowered to 10 mM and supplemented with 250 mM sucrose). The VHH antibody was finally eluted by cleaving the His14-SUMO-tag using 100 nM S. cerevisiae Ulp1 for 2 hours at room temperature. The eluted VHH antibodies were frozen in liquid nitrogen and stored an −80° C. until further use.

### Example 3—Cytoplasmic Expression and Purification of VHH Trimers

The VHH trimers listed in Table 4 were expressed and purified as described for the VHH monomers, the only difference being that the VHH sequence was C-terminally extended by a 31 residues long spacer (in single letter code: EGSEGPESSDGSDSTDPGEQGEGADASDGSE) (SEQ. ID NO: 209) and by the 57 residues long human collagen XVIII NC trimerization domain (in single letter code: GASSGVRLWATRQAMLGQVHEVPEGWLIFVAEQEELYVRVQNGFRKVQLEARTP LPR) (SEQ. ID NO: 210).

### Example 4—Spike Protein-Staining in Transfected Cells

To prepare fluorophore-labelled probes, monomeric VHH antibodies were expressed with two additional cysteine residues, one at the N- and one at the C-terminus. These were then used for labelling with fluorophore-maleimides as described by Pleiner et al., 2015. The trimerized VHH antibody Re6A11 was labelled through a single N-terminal cysteine. Labelings were essentially quantitative as judged by ratiometric UV-VIS spectroscopy and electrophoretic size shifts.

HeLa cells were cultivated in DMEM+ 5% FCS, seeded in 10-well slides, and transiently transfected with a plasmid allowing expression of the SARS-CoV-2 spike protein from a CMV promoter and using the PolyJet transfection reagent according to the manufacturer's instruction (SignaGen). 2 days later, cells were fixed for 5 minutes with 4% paraformaldehyde (PFA) in PBS, washed in PBS, permeabilized with 0.5% saponin, blocked with 5% BSA in PBS, and incubated with gentle shaking for 60 min with fluorophore-labelled VHH antibodies (in PBS+ 1% BSA) at concentrations given in the figures. Following washes with PBS and counterstaining with DAPI, images were taken by confocal laser scanning microscopy.

### Example 5—Spike Protein Detection in SARS-CoV-2 Infected Cells

Stainings were performed with Vero E6 cells infected for 2 days as described above. Fixation was with 4% paraformaldehyde (PFA) for 2 hours. This long period of fixation was due to safety reasons to make sure that no infectious material remained. As a consequence, only highly fixation-resistant epitopes remain visible—explaining the difference between the staining of transfected and virus-infected cells in Table 1. The fluorescence staining themselves were performed as described above. FIG. 3 shows epifluorescence images. The evaluations of Table 1 are based on confocal laser scans.

### Example 6—Virus Infection and Neutralization Assays

Virus stocks were prepared as supernatants from Vero E6 cells infected with SARS-CoV-2. The virus stocks contained between 10¹¹ and 10¹² copies of the virus genome per ml, as determined by reverse transcription and quantitative PCR (qRT-PCR) according to a standard protocol (Corman et al., 2020). 2*10⁶ virus copies were diluted in 100 μl of cell culture media, DMEM/2% FBS, in the presence or absence of varying concentrations of the VHH antibodies under study. After 60 min incubation at 37° C., this was added to a monolayer of Vero E6 cells, i.e. 5000 cells in a well of a standard 96 well cell culture plate. After 48 or 72 hours of incubation, the supernatants of the cells were harvested. To quantify the amount of newly produced virus particles, the RNA from this material was isolated by the Trizol method, followed by qRT-PCR. Alternatively, cells were fixed as in Example 5, stained with a cocktail Atto488 labelled anti-RBD VHHs (10-15 nM each) and with 15 nM Atto565-labelled VHH (Re8H11) that recognizes an S1 SARS-CoV-2 epitope outside the RBD. Imaging was by confocal laser scanning or spinning disc fluorescence microscopy. Neutralization was considered as successful if no infected cells were detectable within the well.

### Example 7—Producing Trimeric VHH Antibodies by Secretion from Pichia pastoris

The sequences of VHH trimers were cloned into a vector that was derived from pPICZα (Invitrogen/ThermoFisher Scientific, Cat. No. V19520) by replacing the signal peptide of the mating factor α precursor by the signal peptide of ScOst1p (Barrero et al., 2018) and replacing the zeocin resistance cassette by a coding sequence for the aminoglycoside 3′ phosphotransferase (UniProt ID: KKA1_ECOLX), conferring resistance to kanamycin and G418. The plasmids encoding VHH antibody trimers were transformed into wild-type P. pastoris cells using the protocol described by (Wu and Letchworth, 2004) with the following modifications: 2 μg of linearized vector DNA were used per transformation. After electroporation by pulse at 1.5 kV in a 2 mm electroporation cuvette, cells were allowed to recover in YPDS medium for 1 hour at 30° C. The selection was done directly for resistance to 1 mg/ml of G418 on YPDS plates for 60-80 hours.

Resulting clones were screened for expression of a desired protein by inoculating a single colony into BMMY medium in a well of a deep-well plate and incubating at 28° C. shaking at 500 rpm for 48 hours and analysing the resulting culture medium by SDS-PAGE. The best clones were used for medium scale expression by growing a culture of the desired strain in BMGY at 28° C. shaking at 120 rpm to mid-log phase, harvesting the cells and resuspending them in BMMY to induce expression under control of AOX1 promoter and incubating a culture at 28° C. shaking at 120 rpm. Additional methanol was supplied 24 hours post-induction to replenish consumed/evaporated methanol. After 48 hours post-induction, cells were removed from the culture by centrifugation at 3000 g for 10 minutes, then at 10000 g for 10 minutes. The final supernatant was filtered through a 0.2 μm filter before proceeding to VHH purification.

### Example 8—Highly Potent Neutralizers of the South African B.1.351 SARS-CoV-2 Strain (Beta Variant)

In a further set of experiments, the inventors assessed the neutralization potency of their mutation-tolerant VHH antibodies, using the South African B.1.351 variant as a representative of the recently emerged mutant strains. As their microscopic assay relied on VHH antibody-based detection of newly made viral proteins, they had to ensure that the mutant spike also yields an unambiguous signal. This was achieved with a mix of VHH-antibodies against epitopes outside the RBD (anti-S1ΔRBD), against the non-mutated epitope 2 (Re7E02 or Re9C07), and by the mutation-tolerant main epitope-binder Re9H03.

By contrast, Re6D06, which fails in mutant RBD binding, also failed to stain mutant spikes of infected cells suggesting that combinations of mutation-sensitive and -tolerant VHH-antibodies can diagnose virus variants by simple staining procedures.

The inventors then compared two strategies for the actual neutralization of mutant B.1.351 (FIG. 9). They first tested the most promising VHH monomers and observed potent neutralization by Re5F10 (at 1.7 nM), Re6H06 (at 170 pM), Re9B09 (at 1.7 nM), and by the mutant-preferring Re9B09 class member Re9H03 (at 50-170 pM; FIG. 9A, Table 5). Even more potent B.1.351 neutralization was found for the heterodimers Re9F06-R28 (50 pM), Re9F06-Re9B09 (50 pM), and Re9F06-Re6H06 (17 pM; FIG. 9B), as well as for the analogous Re21D01-heterodimers with R28, Re9B09 and Re6H06, whereby VHH-antibody-connecting linkers of 14, 15, or 29 amino acids gave rather similar results (for additional data, see Table 5 for VHH monomers and Table 6 for VHH heterodimers).

TABLE 5

| | | Lowest concentration |
|---|---|---|
| VHH monomer | SEQ ID | neutralizing B.1.351 (nM) |
| Re5F10 | 29 | 1.7-5 |
| Re21D01 (Re5F10 class) | 260 | 5 |
| Re26E09 (Re5F10 class) | 284 | 1.7-5 |
| Re26E11 (Re5F10 class) | 288 | 1.7-5 |
| Re21H01 | | 5 |
| Re6H06 | 81 | 0.170 |
| Re26D07 (Re6H06 class) | 280 | 0.500 |
| Re9B09 | 129 | 1.7 |
| Re9H03 (Re9B09 class) | 252 | 0.050-0.170 |

Neutralization of the South African B.1.351 SARS-CoV-2 variant by mutation-tolerant VHH antibodies.

TABLE 6

| | | | | Lowest neutralizing concentration (pM) | | |
|---|---|---|---|---|---|---|
| | | | | | | Beta |
| | | | | | Alpha | South |
| | | | | Wild | UK | African |
| VHH1 | Spacer | VHH2 | Expression | type | B.1.1.7 | B.1.351 |
| Re9F06 | A | Re9B09 | E. coli | 50-170 | | 17-170 |
| Re9F06 | A | Re5D06R28D | E. coli | 5-50 | | 5-170 |
| Re22D04 | A | Re5D06R28D | E. coli | | | 170 |
| Re25H10 | A | Re9B09 | E. coli | 500 | 50-170 | 170 |
| Re25H10 | A | Re5D06R28D | E. coli | 170 | 170 | 170 |
| Re9F06 | B | Re5D06R28D | Pichia | | | 50 |
| Re9F06 | C | Re6H06 | Pichia | 500 | 50 | 170 |
| Re9F06 | C | Re9B09 | Pichia | 17-170 | 170 | 50-170 |
| Re9F06 | B | Re6H06 | Pichia | 17-170 | | 50 |
| Re9F06 | B | Re9B09 | Pichia | 50-170 | | 17-170 |
| Re9F06 | B | Re5D06R15_3QE | Pichia | 5-50 | | 50-170 |
| Re9F06 | B | Re5D06R28_3QE | Pichia | | | 50-170 |
| Re21D01 | B | Re5D06R15_3QE | Pichia | 50-170 | | |
| Re21D01 | B | Re5D06R28_3QE | Pichia | 17-50 | | 50-170 |
| Re21D01 | C | Re5D06R15_3QE | Pichia | 50 | 170 | |
| Re21D01 | C | Re5D06R28_3QE | Pichia | 50 | 50 | |
| Re21D01 | B | Re6H06 | Pichia | 50 | | 50-500 |
| Re21D01 | B | Re9B09 | Pichia | 50 | | |
| Re21D01 | C | Re6H06 | Pichia | 170 | 50-500 | 50-170 |
| Re21D01 | C | Re9B09 | Pichia | 50-170 | 170 | |

Neutralization of the South African B.1.351 SARS-CoV-2 variant by VHH-spacer-VHH tandem fusions. Spacer A: TSGEGEGGEGGEGS (SEQ. ID NO: 312); Spacer B: TSGEGEGGGEGGEGS (SEQ. ID NO: 313); Spacer C: TSGEGEGGGEGGSGEGGSEGGEGGSGEGS (SEQ. ID NO: 314).

### Example 9: Binding of Mutation-Tolerant Monomeric VHHs and Heterodimeric VHHs to the RBD of the SARS-CoV-2Delta Variant

In further experiments, the inventors used Biolayer interferometry (BLI) to assess the binding strength of selected VHHs to the receptor-binding domain of the Delta variant of SARS-CoV-2, which carries the L452R and T478K mutations. For this, VHH constructs were labelled with Biotin-PEG₁₁-Maleimide (#PEG1595; Iris Biotech) through N- and C-terminally introduced cysteines.

BLI experiments were performed using High Precision Streptavidin biosensors and an Octet RED96e instrument (ForteBio/Sartorius) at 25° C. with Phosphate-Buffered Saline (PBS) pH 7.4, 0.02% (w/v) Tween 20 and 0.1% (w/v) BSA as assay buffer.

The indicated VHHs were immobilized on sensors until a binding signal of 0.4 nm (for monomeric VHHs) or 0.75 nm (for heterodimeric VHHs) was reached. Subsequently, the biosensors were dipped into wells containing 3-fold dilutions (20, 6.66, and 2.22 nM) of the wild type RBD (Z03479, GenScript) or the B.1.617.2/Delta RBD (40592-V08H90, Sino Biologicals) for 450 s. RBD dissociation in assay buffer was followed for 900 s. Data were reference-subtracted and curves were fitted using a mass transport model (Octet Data Analysis HT 12.0 software). The dissociation constants (K_{D}s) are summarized in the Table shown below. They revealed very tight binding of the analyzed VHHs and heterodimeric VHHs.

TABLE 7

| | | K_{D} RBD | | K_{D} RBD | |
|---|---|---|---|---|---|
| VHH monomer or VHH heterodimer | SEQ ID | Wild type | | Delta variant | |
| Re5F10 | 29 | 40 | pM | 40 | pM |
| Re6H06 | 81 | ≤10 | pM | ≤10 | pM |
| Re9B09 | 129 | ≤10 | pM | 100 | pM |
| Re5D06R15 | 224 | ≤10 | pM | 30 | pM |
| Re5D06R28 | 232 | ≤10 | pM | 80 | pM |
| Re9F06-spacerA-Re9B09 | 292 | ≤10 | pM | ≤10 | pM |
| Re9F06-spacerA-Re5D06R28D | 293 | ≤10 | pM | ≤10 | pM |

### Example 10: Neutralization of the SARS-CoV-2 Delta Variant by Mutation-Tolerant VHHs and VHH Tandems

The indicated VHH constructs were used in neutralization experiments of the Delta SARS-CoV-2 variant. Experiments were performed analogously to those described in FIGS. 5 and 9. Table 8 lists the lowest neutralizing concentrations as geometric means of three independent experiments.

TABLE 8

| | | Lowest neutralizing |
|---|---|---|
| | SEQ | concentration |
| VHH monomer or VHH heterodimer | ID | Delta variant (nM) |
| Re6H06 | 81 | 0.5 |
| Re9B09 | 129 | 1.7 |
| Re9F06-SpacerB-Re5D06R28D | 297 | 0.05 |
| Re9F06-SpacerB-Re5D06R15_3QE | 302 | 0.09 |
| Re9F06-SpacerB-Re5D06R28_3QE | 303 | 0.09 |
| Re21 D01-SpacerC-Re5D06R28_3QE | 307 | 0.29 |
| Re21D01-SpacerB-Re5D06R28_3QE | 305 | 0.29 |
| Re9F06-SpacerC-Re9B09 | 299 | 0.29 |
| Re9F06-SpacerB-Re6H06 | 300 | 0.50 |
| Re9F06-SpacerC-Re6H06 | 298 | 0.50 |
| Re21D01-SpacerC-Re6H06 | 310 | 0.50 |

### Example 11: The Trimerization Module of Collagen XVIII Allows for More Potent SARS-CoV-2 Neutralizing VHH Fusions than the Homologous NC1 Domain of Collagen XV

The indicated trimeric VHH fusions were expressed in E. coli, purified and tested in a SARS-CoV-2 neutralization assay as described in FIGS. 3 and 5.

The lowest neutralizing concentrations (referring to VHH moieties) are listed in Table 9. The collagen XVIII NC1 domain was used as a C-terminal fusion partner. The collagen XV NC1 domain was used an N-terminal fusion partner in Re9B09, Re9H01 und Re7H02 (Re6B06 class) fusions, and as a C-terminal fusion partner in the KG4B11 fusion.

The data shows that the C-terminal collagen XVIII fusion allows for greater potency than an collagen XV NC1 on either the N-terminus or the C-terminus.

TABLE 9

| | Collagen XV | | Collagen XVIII | |
|---|---|---|---|---|
| | NC1 fusion | | NC1 fusion | |
| | | Lowest | | Lowest |
| | | neutralizing | | neutralizing |
| | SEQ | concentration | SEQ | concentration |
| VHH trimer | ID | (pM) | ID | (pM) |
| Re9B09 | 315 | 90 | 316 | 16 |
| Re9H01 | 317 | 50 | 318 | 4 |
| Re7H02 (Re6B06 class) | 319 | 160 | 212 | 4 |
| KG4B11 (Re6B06 class) | 320 | 170 | 213 | 4 |

Further examples are detailed in the figures and figure legends.

### REFERENCES

Alvarez-Cienfuegos, A., Nuñez-Prado, N., Compte, M., Cuesta, A. M., Blanco-Toribio, A., Harwood, S. L., Villate, M., Merino, N., Bonet, J., Navarro, R., Muñoz-Briones, C., Sørensen, K. M., Mølgaard, K., Oliva, B., Sanz, L., Blanco, F. J., and Alvarez-Vallina, L. (2016). Intramolecular trimerization, a novel strategy for making multispecific antibodies with controlled orientation of the antigen binding domains. Sci Rep 6, 28643.
Arbabi Ghahroudi, M., Desmyter, A., Wyns, L., Hamers, R., and Muyldermans, S. (1997). Selection and identification of single domain antibody fragments from camel heavy-chain antibodies. FEBS Lett 414, 521-526.
Bar-On, Y. M., Flamholz, A., Phillips, R., and Milo, R. (2020). SARS-CoV-2 (COVID-19) by the numbers. eLife 9,
Barrero, J. J., Casler, J. C., Valero, F., Ferrer, P., and Glick, B. S. (2018). An improved secretion signal enhances the secretion of model proteins from Pichia pastoris. Microb Cell Fact 17, 161.
Bogin, O., Kvansakul, M., Rom, E., Singer, J., Yayon, A., and Hohenester, E. (2002). Insight into Schmid metaphyseal chondrodysplasia from the crystal structure of the collagen X NC1 domain trimer. Structure 10, 165-173.
Boudko, S. P., Sasaki, T., Engel, J., Lerch, T. F., Nix, J., Chapman, M. S., and Bächinger, H. P. (2009). Crystal structure of human collagen XVIII trimerization domain: A novel collagen trimerization Fold. J Mol Biol 392, 787-802.
Chen, C., Zhang, Y., Huang, J., Yin, P., Cheng, Z., Wu, J., Chen, S., Zhang, Y., Chen, B., Lu, M., Luo, Y., Ju, L., Zhang, J., and Wang, X. (2020a). Favipiravir versus Arbidol for COVID-19: A Randomized Clinical Trial.
Chen, X., Zhao, B., Qu, Y., Chen, Y., Xiong, J., Feng, Y., Men, D., Huang, Q., Liu, Y., Yang, B., Ding, J., and Li, F. (2020b). Detectable serum SARS-CoV-2 viral load (RNAaemia) is closely correlated with drastically elevated interleukin 6 (IL-6) level in critically ill COVID-19 patients. Clin Infect Dis
Chug, H., Trakhanov, S., Hülsmann, B. B., Pleiner, T., and Görlich, D. (2015). Crystal structure of the metazoan Nup62⋅Nup58⋅Nup54 nucleoporin complex. Science 350, 106-110.
Corman, V. M., Landt, O., Kaiser, M., Molenkamp, R., Meijer, A., Chu, D. K., Bleicker, T., Brünink, S., Schneider, J., Schmidt, M. L., Mulders, D. G., Haagmans, B. L., van der Veer, B., van den Brink, S., Wijsman, L., Goderski, G., Romette, J. L., Ellis, J., Zambon, M., Peiris, M., Goossens, H., Reusken, C., Koopmans, M. P., and Drosten, C. (2020). Detection of 2019 novel coronavirus (2019-nCoV) by real-time RT-PCR. Euro Surveill 25,
Duan, K., Liu, B., Li, C., Zhang, H., Yu, T., Qu, J., Zhou, M., Chen, L., Meng, S., Hu, Y., Peng, C., Yuan, M., Huang, J., Wang, Z., Yu, J., Gao, X., Wang, D., Yu, X., Li, L., Zhang, J., Wu, X., Li, B., Xu, Y., Chen, W., Peng, Y., Hu, Y., Lin, L., Liu, X., Huang, S., Zhou, Z., Zhang, L., Wang, Y., Zhang, Z., Deng, K., Xia, Z., Gong, Q., Zhang, W., Zheng, X., Liu, Y., Yang, H., Zhou, D., Yu, D., Hou, J., Shi, Z., Chen, S., Chen, Z., Zhang, X., and Yang, X. (2020). Effectiveness of convalescent plasma therapy in severe COVID-19 patients. Proceedings of the National Academy of Sciences 117, 9490-9496.
Emeny, J. M., and Morgan, M. J. (1979). Regulation of the interferon system: evidence that Vero cells have a genetic defect in interferon production. J Gen Virol 43, 247-252.
Hoefman, S., Ottevaere, I., Baumeister, J., and Sargentini-Maier, M. (2015). Pre-Clinical Intravenous Serum Pharmacokinetics of Albumin Binding and Non-Half-Life Extended Nanobodies®. Antibodies 4, 141-156.
Kliks, S. C., Nisalak, A., Brandt, W. E., Wahl, L., and Burke, D. S. (1989). Antibody-dependent enhancement of dengue virus growth in human monocytes as a risk factor for dengue hemorrhagic fever. Am J Trop Med Hyg 40, 444-451.
Li, W., Moore, M. J., Vasilieva, N., Sui, J., Wong, S. K., Berne, M. A., Somasundaran, M., Sullivan, J. L., Luzuriaga, K., Greenough, T. C., Choe, H., and Farzan, M. (2003). Angiotensin-converting enzyme 2 is a functional receptor for the SARS coronavirus. Nature 426, 450-454.
Littaua, R., Kurane, I., and Ennis, F. A. (1990). Human IgG Fc receptor II mediates antibody-dependent enhancement of dengue virus infection. J Immunol 144, 3183-3186.
Magro, C., Mulvey, J. J., Berlin, D., Nuovo, G., Salvatore, S., Harp, J., Baxter-Stoltzfus, A., and Laurence, J. (2020). Complement associated microvascular injury and thrombosis in the pathogenesis of severe COVID-19 infection: a report of five cases. Transl Res
Mehta, P., McAuley, D. F., Brown, M., Sanchez, E., Tattersall, R. S., Manson, J. J., and HLH Across Specialty Collaboration, U. K. (2020). COVID-19: consider cytokine storm syndromes and immunosuppression. Lancet 395, 1033-1034.
Murphy, K., and Weaver, C. (2016). Janeway's Immunobiology Garland Science).
Pleiner, T., Bates, M., and Görlich, D. (2018). A toolbox of anti-mouse and anti-rabbit IgG secondary nanobodies. J Cell Biol 217, 1143-1154.
Pleiner, T., Bates, M., Trakhanov, S., Lee, C. T., Schliep, J. E., Chug, H., Böhning, M., Stark, H., Urlaub, H., and Görlich, D. (2015). Nanobodies: site-specific labeling for super-resolution imaging, rapid epitope-mapping and native protein complex isolation. Elife 4, e11349.
Rasmussen, S. G., Choi, H. J., Fung, J. J., Pardon, E., Casarosa, P., Chae, P. S., Devree, B. T., Rosenbaum, D. M., Thian, F. S., Kobilka, T. S., Schnapp, A., Konetzki, I., Sunahara, R. K., Gellman, S. H., Pautsch, A., Steyaert, J., Weis, W. I., and Kobilka, B. K. (2011). Structure of a nanobody-stabilized active state of the β(2) adrenoceptor. Nature 469, 175-180.
Stegmann, K. M., Dickmanns, A., Gerber, S., Nikolova, V., Klemke, L., Manzini, V., Schlösser, D., Bierwirth, C., Freund, J., Sitte, M., Lugert, R., Salinas, G., Görlich, D., Wollnik, B., Groß, U., and Dobbelstein, M. (2020). The folate antagonist methotrexate diminishes replication of the coronavirus SARS-CoV-2 and enhances the antiviral efficacy of remdesivir in cell culture models. bioRxiv 2020.07.18.210013.
Wang, C., Li, W., Drabek, D., Okba, N. M. A., van Haperen, R., Osterhaus, A. D. M. E., van Kuppeveld, F. J. M., Haagmans, B. L., Grosveld, F., and Bosch, B. J. (2020). A human monoclonal antibody blocking SARS-CoV-2 infection. Nat Commun 11, 2251.
Wirz, J. A., Boudko, S. P., Lerch, T. F., Chapman, M. S., and Bächinger, H. P. (2011). Crystal structure of the human collagen XV trimerization domain: A potent trimerizing unit common to multiplexin collagens. Matrix Biology 30, 9-15.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
Wu, S., and Letchworth, G. J. (2004). High efficiency transformation by electroporation of Pichia pastoris pretreated with lithium acetate and dithiothreitol. Biotechniques 36, 152-154.
Zhang, L., Jackson, C. B., Mou, H., Ojha, A., Rangarajan, E. S., Izard, T., Farzan, M., and Choe, H. (2020). The D614G mutation in the SARS-CoV-2 spike protein reduces S1 shedding and increases infectivity. bioRxiv

1. A homotrimeric VHH antibody comprising 3 VHH antibody subunits recognizing the same target structure, wherein each VHH antibody subunit comprises a VHH antibody directly linked or linked via a spacer to a trimerization module.

2. The homotrimeric VHH antibody of claim 1, which comprises three identical VHH antibody subunits.

3. The homotrimeric VHH antibody of claim 1 wherein the VHH antibody is directed to a target structure having a threefold-rotationally symmetric subunit structure.

4. The homotrimeric VHH antibody of claim 1, wherein the VHH antibody is directed to a target structure comprising a homotrimeric protein or protein assembly.

5. The homotrimeric VHH antibody of claim 1, wherein the target structure is a viral protein or protein assembly, particularly a homo-trimeric viral protein or protein assembly, more particularly selected from a Coronavirus trimeric spike protein, an Orthomyxovirus trimeric HA protein, a Paramyxovirus trimeric fusion protein, a Dengue fever virus or Zikavirus trimeric or higher order protein assembly, a Herpesvirus trimeric gB fusion protein or a HIV trimeric gp120 protein.

6. The homotrimeric VHH antibody of claim 1, wherein the target structure is the SARS-CoV-2 spike protein S1 domain, particularly the receptor-binding domain (RBD) of the SARS-CoV-2 S1 domain including the RBD of wildtype SARS-CoV-2 and the RBD domain of a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.

7. The homotrimeric VHH antibody of claim 6, which is a neutralizing trimeric VHH antibody, e.g. a VHH antibody which has a lowest SARS-CoV-2 neutralizing concentration of about 100 pM or less, of about 25 pM or less, of about 17 pM or less, of about 10 pM or less, of about 5 pM or less or of about 1.7 pM or less.

8. The homotrimeric VHH antibody of claim 6, wherein the VHH antibody is selected from the VHH antibody comprising
(a) a CDR3 sequence as shown in SEQ. ID NO: 20, 204, 208, 4, 8, 12, 16, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 227, 231, 235, 239, 243, 247, 251, 255, 259, 263, 267, 271, 275, 279, 283, 287, or 291,
(b) a CDR3 sequence which has an identity of at least 80%, at least 90%, or at least 95% to a CDR3 sequence of (a), or
(c) a VHH antibody, which competes with a VHH antibody of (a) for the binding to the SARS-CoV-2 spike protein S1 domain, particularly the receptor-binding domain (RBD) of the SARS-CoV-2 S1 domain.

9. The homotrimeric VHH antibody of claim 6, wherein the VHH antibody is selected from the VHH antibody comprising
(a) a combination of CDR1, CDR2 and CDR3 sequences as shown in SEQ. ID NO: 18-20, 202-204, 206-208, 2-4, 6-8, 10-12, 14-16, 22-24, 26-28, 30-32, 34-36, 38-40, 42-44, 46-48, 50-52, 54-56, 58-60, 62-64, 66-68, 70-72, 74-76, 78-80, 82-84, 86-88, 90-92, 94-96, 98-100, 102-104, 106-108, 110-112, 114-116, 118-120, 122-124, 126-128, 130-132, 134-136, 138-140, 142-144, 146-148, 150-152, 154-156, 158-160, 162-164, 166-168, 170-172, 174-176, 178-180, 182-184, 186-188, 190-192, 194-196, 198-200, 225-227, 229-231, 233-235, 237-239, 241-243, 245-247, 249-251, 253-255, 257-259, 261-263, 265-267, 269-271, 273-275, 277-279, 281-283, 285-287, or 289-291,
(b) a combination of CDR1, CDR2 and CDR3 sequences which has an identity of at least 80%, at least 90%, or at least 95% to a combination of CDR1, CDR2 and CDR3 sequences of (a), or
(c) a VHH antibody, which competes with a VHH antibody of (a) for the binding to the SARS-CoV-2 spike protein S1 domain, particularly the receptor-binding domain (RBD) of the SARS-CoV-2 S1 domain.

10. The homotrimeric VHH antibody of claim 6, wherein the VHH antibody is selected from the VHH antibody comprising
(a) a VHH sequence as shown in SEQ ID NO. 17, 201, 205, 1, 5, 9, 13, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 224, 228, 232, 236, 240, 244, 248, 252, 256, 260, 264, 268, 272, 276, 280, 284 or 288,
(b) a sequence which has an identity of at least 70%, at least 80%, at least 90%, at least 95%, or at least 99% to a VHH sequence of (a), or
(c) VHH antibody, which competes with a VHH antibody of (a) for the binding to the SARS-CoV-2 spike protein S1 domain, particularly the receptor-binding domain (RBD) of the SARS-CoV-2 S1 domain.

11. The homotrimeric VHH antibody of claim 6, wherein the VHH antibody is selected from:
(a) the VHH antibody VHH-72 comprising an VHH amino acid sequence as shown in SEQ: ID NO. 217, or
(b) a sequence which has an identity of at least 70%, at least 80%, at least 90%, at least 95% or at least 99% to a VHH sequence of (a).

12. The homotrimeric VHH antibody of claim 6, which is capable of virus neutralization, and which is particularly capable of neutralization of a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants.

13. The homotrimeric VHH antibody of claim 1, which is stable.

14. The homotrimeric VHH antibody of claim 13, which has a melting temperature of at least about 65° C., of at least about 80° C., of at least 90° C. or of at least about 95° C. when measured under non-reducing conditions and/or under reducing conditions.

15. The homotrimeric VHH antibody of claim 1, wherein the target structure is different from spike protein S1 domain, particularly from the receptor-binding domain (RBD) of the SARS-CoV-2 S1 domain.

16. The homotrimeric VHH antibody of claim 1, wherein the target structure is a non-viral protein or protein assembly, particularly a member of the TNF Ligand superfamily or a member of the TNF Receptor superfamily.

17. The homotrimeric VHH antibody of claim 1, wherein each subunit comprises a VHH antibody linked via a spacer to a trimerization module.

18. The homotrimeric VHH antibody of claim 17, wherein the spacer has a length of about 5 to about 100 amino acids, particularly of about 10 to about 50 amino acids.

19. The homotrimeric VHH antibody of claim 17, wherein the spacer comprises amino acids selected from Gly, Ser, Ala, Thr, Pro, Asp and Glu, particularly in an amount of at least about 50%, about 60%, about 70%, about 80%, about 90% or 100%.

20. The homotrimeric VHH antibody of claim 17, wherein the spacer comprises amino acids selected from Gly, Glu, Ser and Pro particularly in an amount of at least about 50%, about 60%, about 70%, about 80%, about 90% or 100%.

21. The homotrimeric VHH antibody of claim 17, wherein the spacer comprises a succession of tripeptides, wherein the first and the second amino acid are selected from Gly, Ser, Ala, Thr, and Pro, and the third amino acid is selected from Asp and Glu.

22. The homotrimeric VHH antibody of claim 17, wherein the spacer is substantially free or free of amino acids selected from Asn, Lys, Arg, Trp, Tyr, Phe, Met, Leu, lie, Val, His and Cys.

23. The homotrimeric VHH antibody of claim 1, wherein the trimerization module is located N-terminal to the VHH-antibody.

24. The homotrimeric VHH antibody of claim 1, wherein the trimerization module is located C-terminal to the VHH-antibody.

25. The homotrimeric VHH antibody of claim 1, wherein the trimerization module is selected from:
(a) a collagen trimerization domain, e.g. the NC1 domain of collagen XV, the NC1 domain of collagen XVIII or the NC1 domain of collagen X, or the lung surfactant protein D.
(b) a trimerization domain which has an identity of at least 80%, particularly at least 90%, more particularly at least 95% to a trimerization domain of (a).

26. The homotrimeric VHH antibody of claim 1, wherein the trimerization module is the NC1 domain of collagen XVIII or a trimerization domain which has an identity of at least 80%, particularly at least 90%, more particularly at least 95% thereto.

27. The homotrimeric VHH antibody of claim 1, wherein the trimerization module is the NC1 domain of collagen XVIII or a trimerization domain which has an identity of at least 80%, particularly at least 90%, more particularly at least 95% thereto, located C-terminal to the VHH-antibody.

28. A subunit of a homotrimeric VHH antibody of claim 1.

29. A nucleic acid molecule encoding a homotrimeric VHH antibody according to claim 1 or a subunit thereof, preferably in operative linkage with a heterologous expression control sequence.

30. A vector comprising a nucleic acid molecule according to claim 29.

31. A recombinant cell or non-human organism transformed or transfected with a nucleic acid molecule according to claim 29 or a vector comprising a nucleic acid molecule according to claim 29.

32. The cell or organism of claim 31, which is selected from a bacterium such as E. coli, Bacillus sp., a unicellular eukaryotic organism, e.g. yeast such as Pichia pastoris, Saccharomyces cerevisiae, or Hansenula polymorpha, or Leishmania, an insect cell, a mammalian cell or a plant cell.

33. The homotrimeric VHH antibody of claim 1, or a subunit thereof, which is non-glycosylated.

34. The homotrimeric VHH antibody of claim 1 or a subunit thereof, which is produced in a bacterium, e.g. E. coli.

35. The homotrimeric VHH antibody of claim 1 or a subunit thereof, which is produced in a yeast, e.g. Pichia pastoris.

36. The homotrimeric VHH antibody of claim 1 or a subunit thereof, which is conjugated to one or several polymer moieties, preferably hydrophilic polymer moieties, such as polyethylene glycol (PEG).

37. A method for preventing or treating a disorder caused by and/or associated with an infection with SARS-CoV-2 including an infection with a SARS-CoV-2 escape mutant including the British mutant (Alpha), the South African mutant (Beta), Brazilian mutant (Gamma), the Indian mutant (Delta), the Californian mutant (Epsilon) as well as mutants comprising at least one of the amino acid substitutions in any one of the above mutants, comprising administering a homotrimeric VHH antibody of claim 1 or a subunit thereof.

38. A method for recombinant production of a homotrimeric VHH antibody of claim 1, comprising cultivating a cell or an organism transformed or transfected with a nucleic acid molecule encoding a homotrimeric VHH antibody according to claim 1 or a subunit thereof, or a vector comprising a nucleic acid molecule encoding a homotrimeric VHH antibody according to claim 1 or a subunit thereof; in a suitable culture medium and obtaining the homotrimeric VHH antibody from the cell or organism or from the culture medium.

39. The method of claim 38, comprising cultivating a yeast such as Pichia pastoris Saccharomyces cerevisiae, or Hansenula polymorpha, and obtaining the homotrimeric VHH antibody from the medium.

40. A method for the recombinant production of a monomeric or multimeric VHH antibody, comprising:
cultivating a yeast such as Pichia pastoris, Saccharomyces cerevisiae, or Hansenula polymorpha, comprising a nucleic acid molecule encoding the VHH antibody or a subunit thereof, in a suitable culture medium and
obtaining the monomeric or multimeric, e.g. homotrimeric VHH antibody from the culture medium.

41. The method of claim 40 wherein the monomeric or multimeric VHH antibody is selected from a monomeric VHH antibody, or a homomultimeric, particularly a homotrimeric VHH antibody, more particularly a homotrimeric VHH antibody of claim 1.

42. The method of claim 40 wherein the yeast comprises a nucleic acid molecule encoding a polypeptide comprising (from N-terminus to C-terminus) (i) a cleavable co-translational signal sequence (pre-sequence), (ii) optionally a cleavable pro-sequence, which enhances secretion, and (iii) a VHH antibody sequence, particularly a fusion comprising the VHH antibody sequence, optionally a spacer, and a trimerization domain, e.g. the Collagen XVIII NC1 domain.

43. The method of claim 42, wherein the cleavable co-translational signal sequence (i) is selected from the Ost1 signal sequence of Saccharomyces cerevisiae (SEQ. ID NO: 219) or an Ost1 signal sequence from a related yeast species, e.g. from Pichia pastoris (SEQ. ID NO: 220), Schizosaccharomyces pombe (SEQ. ID NO: 221), or Candida albicans (SEQ. ID NO: 222) or a variant of those, e.g. having an amino acid identity of at least 80%, of at least 90% or of at least 95% thereto.

44. The method of claim 40, wherein the cleavable pro-sequence comprising a secretion signal sequence (ii) is selected from the propeptide of S. cerevisiae alpha-factor (SEQ. ID NO: 223) or a variant thereof, having an amino acid identity of at least 80%, of at least 90% or of at least 95%, or another Kex2-cleavable pro-peptide that promotes packaging into COPII vesicles and thus export from the ER.

45. A nucleic acid molecule encoding a polypeptide comprising (from N-terminus to C-terminus) (i) a cleavable co-translational signal sequence (pre-sequence), (ii) optionally a cleavable pro-sequence, which enhances secretion, and (iii) a VHH antibody sequence, e.g., a monomeric VHH antibody, or a subunit of a homomultimeric, e.g. homotrimeric VHH antibody, particularly a fusion comprising the VHH antibody sequence, optionally a spacer, and a trimerization domain, e.g. the Collagen XVIII NC1 domain.

46. A polypeptide encoded by the nucleic acid molecule of claim 45, which comprises (from N-terminus to C-terminus) (i) a cleavable co-translational signal sequence (pre-sequence), (ii) optionally a cleavable pro-sequence, which enhances secretion, and (iii) a VHH antibody sequence, e.g., a monomeric VHH antibody, or a subunit of a homomultimeric, e.g. homotrimeric VHH antibody, particularly a fusion comprising the VHH antibody sequence, optionally a spacer, and a trimerization domain, e.g. the Collagen XVIII NC1 domain.
